(19) United States
(12) Patent Application Publication
(10) Pub. No.: US 2024/0279774 A1
(43) Pub. Date: Aug. 22, 2024

(54) COMPOSITIONS COMPRISING PROTEINS AND METHODS OF USE THEREOF FOR RARE EARTH ELEMENT SEPARATION

(71) Applicants: Lawrence Livermore National Security, LLC, Livermore, CA (US); The Penn State Research Foundation, University Park, PA (US)

(72) Inventors: Dan Mcfarland Park, Dublin, CA (US); Gauthier Deblonde, San Ramon, CA (US); Ziye Dong, Dublin, CA (US); Yongqin Jiao, Pleasanton, CA (US); Joseph Anthony Mattocks, University Park, PA (US); Joseph Alfred Cotruvo, Jr., University Park, PA (US)

(21) Appl. No.: 18/570,543

(22) PCT Filed: Jun. 14, 2022
(86) PCT No.: PCT/US22/33462

§ 371 Date: Dec. 14, 2023

Related U.S. Application Data
(60) Provisional application No. 63/210,311, filed on Jun. 14, 2021.

(51) Int. Cl. C22B 59/00; C22B 3/10; C22B 3/16; C22B 3/18

(52) U.S. Cl. CPC C22B 59/00; C22B 3/10; C22B 3/165; C22B 3/18

(57) ABSTRACT

Methods and materials are provided for the preferential separation of rare earth elements (REEs) from non-REEs in REE-containing materials and separation of certain REEs from other REEs, both individually and in groups. Such methods include steps of (a) providing a protein that can selectively bind one or more REEs; (b) contacting the protein with the REE-containing material, wherein the protein binds at least a portion of the one or more REEs to form one or more protein-REE complexes and an REE-depleted material; (c) separating the one or more protein-REE complexes from at least a portion of the REE-depleted material; and (d) separating the one or more REEs from the protein to produce a purified fraction of the one or more REEs and a regenerated protein.

### CROSS-REFERENCE TO RELATED APPLICATIONS

This application claims the benefit of U.S. Patent Application No. 63/210,311, filed Jun. 14, 2021, the disclosure of which is hereby incorporated by reference in its entirety as if fully set forth herein, including all references and appendices submitted therewith.

### STATEMENT REGARDING FEDERALLY SPONSORED RESEARCH

The United States Government has rights in this application pursuant to Contract No. DE-AC52-07NA27344 between the United States Department of Energy and Lawrence Livermore National Security, LLC for the operation of Lawrence Livermore National Laboratory and Contract No. SC-DE0021007 between United States Department of Energy and Pennsylvania State University.

### BACKGROUND

Rare earth elements (“REEs”) are mined from the Earth's crust. Because of their unique physical and chemical properties, these elements are crucial in a growing number of high-tech products, including high-performance magnets, lasers, computer memory, cell phones, catalytic converters, camera and telescope lenses, and green technologies such as wind turbines and hybrid vehicles, to name a few.

Many countries, including the United States produce REEs, but China has been the predominant producer of REEs, accounting for between 70-90% of the world's supply. REEs are difficult to mine in part because it is unusual to find them in concentrations high enough for economical extraction. Use of GPS-controlled drills and Gamma-ray sampling allows geologists to identify higher REE-containing ore. The ore is often laced with natural radioactive materials such as thorium and current methods for the extraction and processing of REEs requires large amounts of carcinogenic toxins including organic solvents, ammonia salts, alkyl phosphorus-containing extractants, and strong mineral acids. Leaching and separation of metals has high energy/capital costs, high CO₂ emissions, and many negative health and environmental impacts.

As the demand for REEs continues to surge at a rapid rate, there remains a need for tools to help increase and diversify the supply of REEs, develop clean and low-cost extraction processes, improve efficiencies, and recapture REEs through reuse and recycling. In particular, there is a need for the development of tools capable of preferentially separating REEs, especially from REE feedstocks in which the REE content is low relative to non-REEs with a focus on maximizing efficiency and minimizing waste.

### SUMMARY

Methods and materials are provided for the preferential separation of rare earth elements (REEs) from non-REEs in REE-containing materials and separation of certain REEs from other REEs, both individually and in groups. In certain embodiments, such methods include steps of (a) providing a protein that can selectively bind one or more REEs; (b) contacting the protein with the REE-containing material, wherein the protein binds at least a portion of the one or more REEs to form one or more protein-REE complexes and an REE-depleted material; (c) separating the one or more protein-REE complexes from at least a portion of the REE-depleted material; and (d) separating the one or more REEs from the protein to produce a purified fraction of the one or more REEs and a regenerated protein.

In other embodiments, a method for preparing a material for rare earth element (REE) separation is provided. Such a method includes steps of (a) providing a protein that can selectively bind an REE and a porous support material functionalized with a conjugation agent; and (b) conjugating the protein to the porous support material via the conjugation agent.

In certain embodiments, a method of preferentially separating rare earth elements (REE) from a REE-containing material is provided. Said method includes steps of (a) providing a plurality of proteins that can selectively bind one or more REEs; (b) contacting the plurality of proteins with the REE-containing material, wherein the plurality of proteins bind at least a portion of the one or more REEs to form a plurality of protein-REE complexes and an REE-depleted material; (c) separating the plurality of protein-REE complexes from at least a portion of the REE-depleted material; (d) preferentially separating heavy REEs (HREEs) from the plurality of proteins by contacting the plurality of protein-REE complexes with a first solution comprising a chelator or a first solution having a first pH; (g) separating middle REEs (MREEs) from the plurality of proteins by contacting the plurality of protein-REE complexes with a second solution comprising a chelator or a second solution having a second pH; and (h) separating light REEs (LREEs) from the plurality of proteins by contacting the plurality of protein-REE complexes with a third solution comprising a chelator or a third solution having a third pH.

In certain embodiments, a method of preferentially separating scandium and yttrium from a REE-containing material using chelators is provided. Said method includes steps of (a) providing a plurality of proteins that can selectively bind one or more REEs; (b) contacting the plurality of proteins with the REE-containing material, wherein the plurality of proteins bind at least a portion of the one or more REEs to form a plurality of protein-REE complexes and an REE-depleted material; (c) separating the plurality of protein-REE complexes from at least a portion of the REE-depleted material; (d) preferentially separating scandium from the plurality of proteins by contacting the plurality of protein-REE complexes with a first chelator solution, (e) preferentially separating yttrium from the plurality of proteins by contacting the plurality of protein-REE complexes with a second chelator solution; (f) separating heavy (HREEs) and middle REEs (MREEs) from the plurality of proteins by contacting the plurality of protein-REE complexes with a solution comprising a third chelator solution; and (g) separating light REEs (LREEs, here, La and Ce) from the plurality of proteins by contacting the plurality of protein-REE complexes with a fourth chelator solution or a solution having a low pH (<1.7). In certain embodiments, the first solution includes malonate at a concentration of 20-50 mM, the second solution contains citrate at a concentration of about 15.0 mM, the third solution includes citrate at a concentration of about 25 mM to about 50 mM, and the fourth solution has a pH of about 1.5.

In certain embodiments, a method of preferentially separating scandium from a REE-containing material using pH is provided. Said method includes steps of (a) providing a plurality of proteins that can selectively bind one or more REEs; (b) contacting the plurality of proteins with the REE-containing material, wherein the plurality of proteins bind at least a portion of the one or more REEs to form a plurality of protein-REE complexes and an REE-depleted material; (c) separating the plurality of protein-REE complexes from at least a portion of the REE-depleted material; (d) preferentially separating yttrium and HREEs from the plurality of proteins by contacting the plurality of protein-REE complexes with a first pH solution, (e) preferentially separating MREEs and LREEs from the plurality of proteins by contacting the plurality of protein-REE complexes with a second pH solution; (f) separating scandium from the plurality of proteins by contacting the plurality of protein-REE complexes with a solution comprising a third pH solution.

In certain embodiments, a method for separating rare earth elements (REE) from a REE-containing material comprising an absorption/desorption cycle. In such embodiments, the absorption/desorption cycle includes steps of loading a column containing a biosorption material with the REE-containing material, the biosorption material comprising a protein capable of adsorbing REEs present in the REE-containing material to form one or more protein-REE complexes and an REE-depleted material; and separating one or more REEs or one or more groups of REEs from the one or more protein-REE complexes using a desorption process comprising a step of eluting a first REE enriched fraction with a first solution capable of preferential separation of the one or more REEs or one or more groups of REEs from the one or more protein-REE complexes. In some embodiments, the desorption process includes a second step of eluting a second REE enriched fraction from the column with a second solution capable of desorbing the one or more REEs from the one or more protein-REE complexes. In other embodiments, the desorption process includes a third step of eluting a third REE enriched fraction from the column with a third solution capable of desorbing the one or more REEs from the one or more protein-REE complexes. Each of the enriched fraction can have a higher concentration of REEs than the feed solution (i.e., the REE-containing material).

In some embodiments, a second absorption/desorption cycle can be performed after the first adsorption/desorption, cycle. In such embodiments, the second adsorption/desorption, cycle includes steps of loading a column containing the biosorption material with the first REE-enriched fraction to form one or more protein-REE complexes; and separating one or more REEs from the one or more protein-REE complexes using a desorption process comprising a step of eluting a first high-purity REE-enriched fraction from the column with the first solution. In some embodiments, the desorption process of the second cycle includes a second step of eluting a second high purity REE enriched fraction from the column with the second solution. In other embodiments, the desorption process further comprises a third step of eluting a third high purity REE enriched fraction from the column with the third solution.

In certain embodiments, each of the first, second and third solutions are designed to have a particular selected pH, include a chelator, or both in order to accomplish the claimed preferential separation. In some embodiments, the first solution has a first selected pH. In some aspects, the first selected pH can be 3.0 or less, 2.5 or less, 2.0 or less, or about 1.5 or less. In other embodiments, the first solution includes a chelator. In certain aspects, the chelator is citrate. In some embodiments, the citrate is present in the first solution at a concentration of about 3.0 mM or about 15.0 mM. Where a chelator is used, the pH of such a solution is higher than 3.0, and in some embodiments, the chelator solution has a pH that is higher than 4.0, between about 4 to 7, or above 7.

In some embodiments, the second solution comprises a second selected pH. In some embodiments, the second selected pH is lower than the first selected pH. In certain aspects, the second selected pH is 2.5 or less, 2.0 or less, or 1.5 or less. In other embodiments, the second solution includes a chelator. In certain aspects, the chelator is citrate. In some embodiments, the second solution comprises citrate at a concentration greater than the first solution. In some embodiments, the citrate is present in the second solution at a concentration of about 15.0 mM citrate. In some embodiments, the citrate is present in the second solution at a concentration between about 25.0 to about 50.0 mM citrate. In some embodiments, the citrate is present in the second solution at a concentration of about 75.0 mM citrate. Where a chelator is used, the pH of such a solution is higher than 3.0, and in some embodiments, the chelator solution has a pH that is higher than 4.0, higher than 5.0, between about 5 to 7, or above 7.

In some embodiments, the first solution and the second solution have a pH between about 2.5 and 1.5. In other embodiments, the first solution comprises a pH between about 2.0 to about 2.5 and the second solution comprises a pH of between about 1.5 to about 2.0. In other embodiments, the first solution comprises a pH of 2.1 and the second solution comprises a pH of 1.7. In some embodiments, the first solution comprises citrate at a concentration of about 3.0 mM and the second solution comprises citrate at a concentration of about 15 mM. In other embodiments, the first solution comprises citrate at a concentration of about 15.0 mM and the second solution comprises citrate at a concentration of about 25 mM to about 50 mM.

In other embodiments, the third solution comprises a third selected pH. In other embodiments, the third selected pH is lower than the second selected pH and the first selected pH is lower than the second selected pH. In other embodiments, the third selected pH is about 1.5 or less or about 1.5. In other embodiments, the third solution includes a chelator selected from EDTA, citrate, dimercaprol, malonate, iminodiacetate, diglycolic acid, a hydroxypyridinone, a hydroxamate, chatecols, polyaminocarboxylates, acetate, nitrilotriacetate, dipicolinic acid, a-hydroxyisobutyric acid, or other mono-, di-, and tri-carboxylic acids. In other embodiments, the third solution includes citrate at a concentration greater than the second solution. In other embodiments, the first solution includes about 75.0 mM citrate.

In other embodiments, the first solution includes citrate at a concentration of about 15.0 mM and the second solution includes citrate at a concentration of about 25 mM to about 50 mM, and the third solution has a pH of about 1.5.

In certain embodiments, the methods described herein also include a step of regenerating the protein using a clearing solution after eluting the REE-enriched fraction or fractions or the high-purity REE-enriched fraction or fractions. In certain embodiments, the cleaning (or clearing) solution comprises a buffer and/or a solution (e.g., HCl or H2SO4 or a mixture HCl/H2SO4) comprising a pH of about 1.0.

In certain embodiments, the protein used in accordance with the disclosures is lanmodulin (LanM). In some embodiments, LanM has an amino acid sequence selected from the group consisting ofSEQ ID NO:1, SEQ ID NO:2, and SEQ ID NO:3. In other embodiments, LanM has an amino acid sequence with at least 95% identity to a sequence selected from the group consisting of SEQ ID NO:1, SEQ ID NO:2, and SEQ ID NO:3.

In some embodiments, a biosorption material is provided. The biosorption material may be composed of microbead for rare earth element (REE) separation that includes a protein that can selectively bind at least one REE, wherein the protein is embedded within or on a surface of the bead. In certain embodiments, the protein is LanM.

In certain embodiments, the biosorption material is composed of amicrobead. In certain embodiments, the beads comprise agarose. In certain embodiments, the concentration of the protein in the bead is at least about 2 μmol, about 3 μmol, about 4 μmol, about 5 μmol, about 6 μmol, about 7 μmol, about 8 μmol, about 9 μmol or about 10 μmol of protein per mL of a total volume the bead. In some embodiments, the LanM includes a C-terminal cysteine residue, a N-terminal cysteine, or an internal cysteine. In some embodiments, the cysteine is linked to the LanM via a glycine-serine-glycine amino acid linker or a hydrophilic linker.

In some embodiments, the separating step preferentially separates one or more individual REEs, groups of REEs, REEs adjacent to each other on the periodic table, REEs having similar ionic radii or combination thereof. In certain embodiments, the protein does not bind non-REE metals in the REE-containing material.

In certain embodiments the REE material is a leachate derived from rare earth ores, geothermal brines, coal, coal byproducts, mine tailings, acid mine drainage, phosphogypsum, end of life products, electronic waste, industrial effluent, or total REE mixtures generated from REE sources, such as salts derived from these ores (e.g. oxide, oxalate, carbonate, etc). In some embodiments, the REE material is an aqueous solution.

### BRIEF DESCRIPTION OF THE DRAWINGS

FIG. 1 shows an exemplary method for separation of REEs using immobilized LanM in accordance with embodiments of the present disclosure.

FIGS. 2A-2D show an exemplary scheme, plots, and images that confirm immobilization of LanM onto agarose microbeads in accordance with embodiments of the present disclosure. Immobilization of LanM onto agarose microbeads through thiol-maleimide click-chemistry (FIG. 2A) was confirmed by Fourier transform infrared spectroscopy (FTIR) of amino-agarose, N-Succinimidyl 4-(maleimidomethyl) cyclohexane-1-carboxylate (SMCC), maleimide-agarose, and LanM-agarose (FIG. 2B), LanM immobilization kinetics (FIG. 2C), and fluorescence microscopy (FIG. 2D, scale bar is 100 m, bottom images are split channels of the top image).

FIGS. 3A-3G show exemplary plots that confirm that immobilized LanM retains the ability to bind REEs at low pH and is stable for reuse in accordance with embodiments of the present disclosure. The plots include a Nd breakthrough curve as a function of pH (FIG. 3A), a Nd desorption curve as a function of HCl concentration (FIG. 3B), plots confirming column reusability based on Nd breakthrough curves with 10 consecutive absorption/desorption cycles (FIG. 3C), independent single element breakthrough curves of Y, La, Nd, Dy and Lu at pH 3 (FIG. 3D), metal ion breakthrough curves using synthetic feed solution to confirm Nd selectivity of LanM column against non-REEs (FIG. 3E), desorption profile of metal ions following treatment with pH 1.5 HCl (FIG. 3F), and a breakthrough experiment using a binary Nd/Fe solution containing citrate to assess the ability of LanM to preferentially separate Nd from Fe (FIG. 3G).

FIGS. 4A-4B show exemplary plots confirming that LanM in solution binds 3 equivalents of REEs in accordance with embodiments of the present disclosure. The plots include a competitive titration assay using xylenol orange as an indicator (FIG. 4A) and a plot showing LanM's conformational response to lanthanum tracked as an increase in the absorbance at A275 nm (FIG. 4B).

FIGS. 5A-5B show exemplary plots that suggest that ligand competition in solution can lead to intra-REE separations in accordance with embodiments of the present disclosure. The plots include stochiometric titrations (FIG. 5A) and REE desorption using citrate (FIG. 5B), monitored by changes in the intrinsic tyrosine fluorescence emission of the protein

FIGS. 6A-6F show exemplary plots that confirm the ability of immobilized LanM to separate pairs of REEs using a two-step pH scheme in accordance with embodiments of the present disclosure. The ability of immobilized LanM to separate pairs of REEs was confirmed by analyzing accumulative desorption profiles of a single element loaded columns using a stepwise pH scheme (FIG. 6A, REE ion desorption was normalized to the total REE desorbed; experimental conditions: independent single REE solutions were used to load column to 90% saturation), a feedstock comprised of a 50:50 mixture of Dy:Nd was loaded to 90% column saturation and then subjected to a two-pH desorption scheme (2.1 and 1.7) (FIG. 6B), a feedstock comprised of a 5:95 mixture of Dy:Nd was loaded to 90% column saturation and then subjected to a two pH desorption scheme (2.2 and 1.7) (FIG. 6C); and a feedstock comprised of a 22:78 mixture of Y:Nd was loaded to 90% column saturation and then subjected to a two-pH desorption scheme (2.3 and 1.7) (FIG. 6D). A summary of REE composition in feedstocks and three desorption zones by using the two-pH scheme is provided in FIG. 6E.

FIGS. 7A-7D show exemplary plots that confirm the ability of immobilized LanM to separate pairs of REEs using a stepwise chelator concentration and pH scheme in accordance with embodiments of the present disclosure. The ability of immobilized LanM to separate pairs of REEs was confirmed by analyzing accumulative desorption profiles of single element loaded columns using a stepwise citrate concentration (pH 5) scheme (FIG. 7A); a feedstock comprised of a 5:95 mixture of Dy:Nd subjected to a two-step desorption scheme using 15 mM citrate (pH 5) followed by pH 1.7 (FIG. 7B); a feedstock comprised of a 50:50 mixture of Dy:Nd was loaded to 90% column saturation and then subjected to a two-step desorption scheme using 10 mM citrate (pH 5) followed by pH 1.7 (FIG. 7C), and a feedstock comprised of a 22:78 mixture of Y:Nd was loaded to 90% column saturation and then subjected to a two-step desorption scheme using 10 mM citrate (pH 5) followed by pH 1.7 (FIG. 7D) (the values above each panel indicate the purity of REE over each elution zone divided by vertical dot lines). A summary of REE composition in feedstocks and three desorption zones by using the citrate-pH scheme is provided at FIG. 6F.

FIGS. 8A-8F show exemplary plots that confirm the ability of immobilized LanM to separate and extract REEs from a low-grade feedstock leachate. The ability of immobilized LanM to separate and extract REEs from a low-grade feedstock leachate was confirmed using a LanM column for REE recovery from a Powder River Basin (PRB) fly ash leachate solution and the results analyzed by adsorption profiles of metal ions (FIG. 8A, adsorption condition: pH 5, 0.5 mL/min. 1 bed volume=0.8 mL), metal compositions in PRB feed and recovered biosorption solutions (FIG. 8B); metal ion percentage in PRB feed and recovered biosorption solutions (excluding monovalent ions) (FIG. 8C), separation factorfortotal REEs relative to selected non-REEs (FIG. 8D), selective desorption of HREE (Tb−Lu+Y) and LREE (La−Gd) by a two-step pH scheme (FIG. 8E), and REE ratio in relation to total REE in PRB feed and three elution zones (FIG. 8F).

FIG. 9 shows an exemplary plot showing desorption profiles of a PRB fly ash leachate by using LanM agarose column (desorption conditions: pH 1.5 HCl).

FIGS. 10A-10B show exemplary plots demonstrating key differences in LanM-REE stability among REEs that forms the basis for REE separation in accordance with embodiments on the present disclosure. The plots include a lanthanide series breakthrough curve using a LanM-agarose column (FIG. 10A, feed: concentration of each REE: 13 μM; pH 3.0, 10 mM glycine) and breakthrough point for individual REEs as a function of ionic radius (FIG. 10B).

FIGS. 11A-11D show exemplary plots demonstrating the efficacy of a LanM column (loaded to 90% capacity) to enable separation of total REEs into HREE, MREE, and LREE fractions when subjected to a stepwise pH desorption scheme in accordance with embodiments on the present disclosure. The plots show a normalized concentration of each REE against bed volume (FIGS. 11A and 11C) and accumulative desorption of each REE against bed volume (FIGS. 11B and 11D). In FIGS. 11A and 11B, the feed composition was as follows: Y: 72.7 μM; La: 53.4 μM; Ce: 113.5 μM; Pr: 13.4 μM; Nd: 51.5 μM; Sm: 10.2 μM; Eu: 2.2 μM; Gd: 9.5 μM; Tb: 1.4 μM; Dy: 7.9 μM; Ho: 1.4 μM; Er: 4.0 μM; Tm: 0.5 μM; Yb: 3.0 μM; Lu: 0.4 μM. In FIGS. 11C and 11D, Scandium was added to the feed composition used in FIGS. 11A and 11B at equivalent concentration to each lanthanide.

FIG. 12 shows an exemplary plot showing LanM affinity to extract Sc in accordance with embodiments on the present disclosure. The plot shows that LanM exhibits a thermodynamic dissociation constant (known as Kd) for Sc of 1.3E-13 M (0.13 μM), at pH 5.

FIG. 13 shows an exemplary plot showing that malonate-induced disassociation of Sc and Lu from LanM in solution is highly selective, and suggesting that Sc can be separated from Lu, the heaviest REE, and therefore likely also the other lanthanides and Y, in accordance with embodiments on the present disclosure (experiment performed with 20 μM protein in 100 mM KCl, 30 mM MOPS, pH 5).

FIG. 14 shows an exemplary plot showing citrate-induced disassociation data for Lu, La, Ce and demonstrating potential to use citrate to separate Lu from Y and MREEs in accordance with embodiments on the present disclosure. (The Nd, Dy, and Y data shown in this figure are the same as in FIG. 5.)

FIGS. 15A-15B show exemplary plots showing that citrate preferentially desorbs scandium, then HREEs, then MREEs, then LREEs from a LanM-agarose column, in ascending order of ionic radius, in accordance with embodiments of the present disclosure. The plots show citrate-chelated REE breakthrough curves using LanM-agarose column [FIG. 15A, feed: equimolar (0.013 mM each) REEs (Sc, Y, La, Ce, Pr, Nd, Sm, Eu, Gd, Yb, Dy, Ho, Er, Tm, Yb, and Lu) in 20 mM citrate at pH 3.5 condition] and stepwise desorption of REEs using different citrate concentrations (FIG. 15B, plot depicts the cumulative desorption of each REE against bed volume). For FIG. 15B, experimental conditions: 21 bed volumes of REEs solution were pumped through LanM column to achieve ˜90% column saturation, followed with 10 bed volumes washing with DI-water. Then a pH 5 desorption solution with citrate concentration steps (3-75 mM) was pumped through the column. Feed: equimolar REEs (Sc, Y, La, Ce, Pr, Nd, Sm, Eu, Gd, Yb, Dy, Ho, Er, Tm, Yb, and Lu) in 10 mM glycine at pH 3.0. Demonstrates efficacy of LanM column (loaded to 90% capacity) to enable separation of total REEs into HREE, MREE, and LREE fractions when subjected to a stepwise citrate desorption scheme.

FIGS. 16A-16B show exemplary plots demonstrating that removal of residues 1-40 of LanM (SEQ ID NO:3) yields a functional protein able to bind REEs in a 2:1, REE to protein, stoichiometric ratio, in accordance with embodiments of the present disclosure. As judged by intrinsic tyrosine fluorescence, LanM α1-40 binds ˜2 equivalents of La and Dy (FIG. 16A) and that LanM α1-40 displays similar (but not identical) solution-state metal disassociation properties (using citrate as desorbant) as wt-LanM, suggesting that the productive EF-hands have been conserved, and similar separation strategies may be applied for this variant (FIG. 16B). The plots include LanM α1-40 fluorometer experiments were the LanM α1-40 variant has had its first, unproductive EF-hand removed, and was tested for REE-binding stoichiometry (FIG. 16A) and citrate-induced metal disassociation (FIG. 16B) at pH 5 in 100 mM KCl, 30 mM MOPS (all experiments performed with 20 μM protein).

FIGS. 17A-17C show exemplary plots demonstrating the stoichiometric REE-binding by the double LanM variant (SEQ ID NO:4) in accordance with embodiments of the present disclosure. The plots include a xylenol orange competition assay suggesting ˜5 equivalents of metal binding exhibiting affinity tighter than micromolar at pH 6 (FIG. 17A), tyrosine absorbance assay suggesting ˜4 equivalents of REE binding (FIG. 17B), and tyrosine fluorescence assay suggesting 4 equivalents of REE binding at pH 5 (FIG. 17C).

FIG. 18 show an exemplary plot demonstrating that metal disassociation characteristics for the double LanM variant are similar to wt-LanM suggesting that double LanM is a productive avenue for increasing column capacity in accordance with embodiments of the present disclosure. The plot includes a citrate-induced metal disassociation curve of double LanM variant's (20 μM) tested at pH 5 in 100 mM KCl, 30 mM MOPS.

FIG. 19 shows an exemplary method for continuous flow separation of REEs using immobilized LanM with a three-column rotation scheme in accordance with embodiments of the present disclosure.

FIG. 20 shows an exemplary plot of metal ion breakthrough curves using LanM-agarose column in accordance with embodiments of the present disclosure (Feed: E-waste leachate generated using biolixiviant at pH 3.5. The biolixiviant was prepared from the supernatant of a Gluconobacter culture and primarily includes gluconic acid and other organic acids as the leaching agents).

FIG. 21. LanM enables production of high purity Nd and Dy, following Nd/Dy separation. A solution comprising a 5:95 mixture of Dy:Nd (pH 3) was subjected to two coupled adsorption/desorption cycles. The first cycle (left panel) generated a high-purity Nd solution (99.9%) and an upgraded Dy (44% Dy/56% Nd) solution. The upgraded Dy solution was used as a feed solution in a second adsorption/desorption cycle (right panel) to generate high-purity Dy and Nd fractions. The duration of each pH step is depicted by the dark gray dashed line and corresponds to the second axis.

FIG. 22. REE recovery and separation from an E-waste bioleachate. (A) Schematic of the stepwise Nd/Pr and Dy recovery and separation process. (B) Relative concentrations (compared to influent) of metals in the effluent. The transitions between steps shown in panel A are depicted by vertical dotted gray lines. The pH value of the strip solutions is noted with the horizontal dotted darker gray line. (C) Absolute REE concentrations eluted from the column. (D) Demonstration of column reusability by column Nd breakthrough curves before and after E-waste test.

FIG. 23. Exemplary Dy vs. Nd/Pr separation from E-waste feedstock. A synthetic solution with an identical composition as the Dy-enriched fraction (29% Dy) from the first extraction step was subjected to a two-step desorption process to generate high-purity Dy and Nd fractions.

FIG. 24. (A) Element composition of a synthetic REE solution used to test the LREE (La+Ce) removal strategy. The REE composition reflects the REE ratio in a typical ore-based leachate solution (e.g., allanite). (B) Accumulative HREE/MREE/LREE desorption profiles of a LanM column loaded with the synthetic REE feedstock depicted in panel A using a stepwise citrate concentration (pH 5) scheme. The REE desorption was normalized to the total REE desorbed in each group.

FIG. 25. (A) Desorption profiles of a REE-loaded column (feedstock composition depicted in FIG. 2A) using a stepwise citrate concentration (Zone I: 15 mM; Zone II: 50 mM at pH 5) and pH (Zone Ill: pH 1.5) scheme. (B) The summary of the REE distribution in the initial feedstock and three desorption regions relative to the total REE in feed solution using citrate-pH scheme.

FIG. 26. Relative REE composition of a synthetic solution that resembles the REE composition in Sc-containing ore (e.g., allanite) and waste product (e.g., bauxite) feedstocks.

FIG. 27. (A) Citrate enables high-purity and high-yield separation of scandium from REEs. Desorption profiles and (B) accumulative desorption (yield) for a REE-loaded LanM column following a stepwise citrate (3 mM and 15 mM at pH 5) and pH 1.5 desorption scheme. (C) REE distribution in the initial feedstock and the three desorption fractions.

FIG. 28. Malonate enables high-purity and high-yield separation of scandium from REEs. A LanM column was loaded with a synthetic REE solution that resembled the REE composition in a typical Sc-containing ore-based feedstock leachate solution. (A) Desorption profiles and (B) Accumulative desorption (yield) following treatment with 30 mM and 50 mM malonate.

FIG. 29. Use of stepwise pH desorption scheme to generate a Sc-enriched fraction. A LanM column was loaded with a synthetic REE solution that resembled the REE composition in a typical Sc containing ore-based feedstock leachate solution. (A) Desorption profiles and (B) Accumulative desorption (yield) and (C) relative composition following treatment with a stepwise pH gradient.

FIG. 30. Distribution factors across the REE series for immobilized LanM, providing a quantitative measure of intra-REE selectivity. The column was washed initially with DI-water, which was then removed by blowing air through the column. Next, 5 mL of feed solution 1 or 2 (total 15 μmol REEs) was circulated through the column at 0.5 mL/min for 2 h and then removed by pushing air through the column. The liquid at equilibrium was collected as [M]ad. Then 4 mL 0.1 M HCl was flowed through the column and effluent was collected as [M]de. Column: WT-LanM (0.9 mL, ˜3.5 μmol/mL Nd adsorption capacity). The column still contains ˜0.8 mL of water when hydrotreated. Free water was removed by blowing air through the column. Solution 1. equal molar (0.33 mM each, total 3 mM) La, Ce, Pr, Nd, Sm, Eu, Gd, Tb, Dy at pH 5. Solution 2. equal molar (0.33 mM each, total 3 mM) Gd, Tb, Dy, Ho, Er, Tm, Yb, Lu, Y at pH 5.

### DETAILED DESCRIPTION

Rare earth elements (REEs), comprised of yttrium, scandium, and the lanthanides, are essential for the transition from the fossil fuel era into the low-carbon era, due to their criticality for clean energy technologies, such as electrical vehicles, wind turbines, and LEDs.¹ Conversely, however, current REE extraction and separation processes require high energy consumption and pose severe environmental burdens that impede the development of a diversified REE supply chain and undercut the environmental benefits of clean energy technologies.^{2,3} To meet the REE demands of the emerging clean energy technology market, it is thus imperative to develop new processing methodologies that enable environmentally friendly REE extraction from REE-bearing resources, low-grade sources and recyclable waste included.

Owing to their similar physicochemical properties and co-occurrence in REE bearing deposits,^{4,5} the separation among REEs is particularly difficult, accounting for ˜30% of the total environmental impact during REE production.^{2,6,7} Currently, REE separation is dominated by chemical-intensive hydrometallurgy processes which involves a primary REE separation from impurities in acid leachate solutions and subsequent group or individual REE separation by liquid-liquid extraction.⁵ In order to reach acceptable enrichment of REEs, liquid-liquid extraction may require hundreds of stages (i.e., mixer-settlers or pulsed columns) that generate a large amount of liquid wastes. Furthermore, it is extremely difficult for liquid-liquid extraction to process variable or dilute REE leachate solutions from unconventional sources, such as industrial wastes and end-of-life consumer electronics (e.g., E-waste), where an enormous amount of leachate solution needs to be in contact with the organic solvent, resulting in large separation units (i.e., high CAPEX) and in losses of solvent dispersed into aqueous systems.

To reduce solvent losses, the liquid-liquid extraction process has been adapted for column chromatography by dissolving REE-selective ligands (extractants) in organic solvent and loading within a solid support.8,9 However, this physical impregnation strategy still inevitably results in leaching of the stationary liquid phase, causing cross-contamination and limited reusability.¹⁰ Solid-liquid extraction (SLE), whereby the chemical ligands (extractant) are covalently anchored onto solid support resin, offers several advantages relative to liquid-liquid extraction,¹¹⁻¹⁴ including faster phase separation between the solid adsorbent and REE bearing solution, significant reduction in the quantity of organic diluent present in the process, and minimal extractant losses.¹⁵ However, most of the SLE adsorbents employed for REE separation are based on existing synthetic organic extractants (ligands) used in liquid-liquid extraction (e.g. HDEHP, TBP, TODGA, or Cyanex derivatives), the selectivity of which, for REE extraction and separation, is usually limited.¹⁶

The incorporation of biological ligands into a SLE process offers the potential for novel chemistries and environmentally sustainable REE separation process development.¹⁷ For example, lanthanide binding tags (LBTs), short peptides that have been engineered for high affinity and selectivity toward REEs, have been displayed on biomaterial surfaces (cells, curli fibers, etc.) and employed in SLE for selective recovery of middle and heavy REEs from various feedstock leachates.^{18,19}However, there still remains a need for methods having selectivity against Zn²⁺, Cu²⁺, and large amounts of Ca²⁺, and increased REE binding below a pH of 5, conditions compatible with REE feedstocks.¹⁸

Of particular relevance for REE extraction, is lanmodulin (LanM), a recently discovered, small (12 kDa) periplasmic protein that is part of a lanthanide uptake and utilization pathway in methylotrophic bacteria.²⁰ Biochemical and biophysical characterization of LanM revealed remarkable selectivity for REEs against other non-REE cations, the ability to bind REEs down to pH ˜2.5, and surprising robustness to repeated acid treatment cycles.¹⁶ Despite the protein's high affinity, desorption can be induced by relatively mild treatments (lowering pH or common chelators like water-soluble carboxylates), and can be cycled to bind and desorb REEs multiple times. Furthermore, LanM also displays an uncommon preference towards middle-light REE over heavy REE; although modest, such a preference might allow for efficient separation of light/heavy REE groups or even important REE pairs.²⁰

To address technical, economic, and environmental limitations of current REE separation approaches, provided herein is a protein-based REE separation method. The protein-based REE separation method is a biomaterial based, all-aqueous REE extraction and separation scheme using the REE-selective LanM protein chelator or its derivatives. To enable facile protein reuse, immobilized LanM is immobilized onto porous agarose microbeads, which are bio-renewable, and commercially available.²¹ The resulting biomaterial allowed effective grouped REE extraction from unconventional, low-grade REE feedstocks. Furthermore, by exploiting the preference of LanM for middle-light REE, the REE separation method described herein provides high purity separation between REE pairs (Nd/Dy, and Y/Nd) and grouped separation between heavy REE (HREE) and light REE (LREE). As such, a key advantage of this approach over liquid-liquid extraction is the combination of primary REE extraction from non-REEs and secondary separation between heavy and light REEs within a single, all-aqueous adsorption/desorption cycle.

After reading this description it will become apparent to one skilled in the art how to implement the invention in various alternative embodiments and alternative applications. However, all the various embodiments of the present invention will not be described herein. It will be understood that the embodiments presented here are presented by way of example only, and not limitation. As such, this detailed description of various alternative embodiments should not be construed to limit the scope or breadth of the present invention as set forth below.

The detailed description is divided into various sections only for the reader's convenience and disclosure found in any section may be combined with that in another section. Titles or subtitles may be used in the specification for the convenience of a reader, which are not intended to influence the scope of the present disclosure.

Unless the context indicates otherwise, it is specifically intended that the various features of the invention described herein can be used in any combination. Moreover, the disclosure also contemplates that in some embodiments any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

### Definitions

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about.” It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth. It also is to be understood, although not always explicitly stated, that the reagents described herein are merely exemplary and that equivalents of such are known in the art.

It must be noted that as used herein and in the appended claims, the singular forms “a,” “an,” and “the” include plural referents unless the context clearly dictates otherwise. Thus, for example, reference to “a protein” includes a plurality of proteins.

As used herein the following terms have the following meanings:

The term “about,” as used herein when referring to a measurable value such as an amount or concentration and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

The phrase “no” or “substantially no” refers to any competing metal that is present in an amount of less than about 0.0001%, less than about 0.001%, less than about 0.01%, less than about 0.1%, less than about 1%, less than about 5%, or less than about 10% of the total weight or volume of the purified REE material, composition, or eluted solution.

The phrase “click chemistry” as used herein refers to a collection of organic reactions that proceed rapidly and selectively under mild conditions to covalently link molecular components.

Also as used herein, “and/or” refers to and encompasses any and all possible combinations of one or more of the associated listed items, as well as the lack of combinations when interpreted in the alternative (“or”).

### Proteins

Aspects of the disclosure provide proteins for use in separating REEs, including the REE-selective lanmodulin (LanM) protein.

Lanmodulin (LanM) is a small protein of around 12 kDa produced by some methylotrophic organisms and is a natural lanthanide-modulated protein. Wild type M. extorquens LanM protein has a sequence of SEQ ID NO:1, and can optionally be N-or C-terminally His-tagged. Methods and composition of the disclosure utilize a LanM protein. Suitable LanM proteins include the wild type M. extorquens LanM protein, or homologs from other organism having at least two EF hand motifs, with at least one EF hand motifs having at least 3 carboxylate residues, and at least 2 of the EF hand motifs being separated by a space of 10-15 residues. Reference herein will be made generally to “lanmodulin,” “LanM” or “LanM protein” and should be understood to include the wild type and homologs described herein. “LanM” can include full proteins having one or more LanM units or portions thereof comprising the one or more LanM units. LanM units include at least two EF hand motifs, with at least one EF hand motifs having at least 3 carboxylate residues, and at least 2 of the EF hand motifs being separated by a space of 10-15 residues. For ease of reference, discussion will be made with reference to lanmodulin, LanM or LanM protein and should be understood to include both the full proteins and portions of full proteins having the suitable LanM unit.

The unique features of the EF hands of this protein have been discussed previously in the initial characterization of the protein (Cotruvo et al., J. Am. Chem. Soc. 2018, 140, 44, 15056-15061). Based on biochemical and structural studies of the M. extorquens AM1 lanmodulin as well as homologs from other organisms, the lanmodulin protein domain has particular key characteristics. In many examples, at least one of the EF hands contained a proline residue at the second position. However, not all LanM homologs possess such a residue; yet they exhibit other key features of LanM that were identified, such as the spacing between at least 2 of the adjacent EF hands (10-15 residues, such as 12-13 residues) and additional carboxylate residues in the EF hands. In particular, the spacing between adjacent EF hands appears to be a hallmark of these proteins that sets them apart from traditional EF hand containing proteins, such as calmodulin. Even sequences with very low (<40%) identity to M. extorquens LanM can possess similar properties if the other general features mentioned above are conserved. In embodiments, the LanM is the M. extorquens wild type LanM having the sequence of SEQ ID NO:1, and can optionally be terminal His-tagged. In embodiments, the LanM can be a homolog of the wild type LanM. Suitable homologs have at least 2 EF hand motifs, with at least one EF hand motif having at least 3 carboxylate residues, and at least 2 of the EF hand motifs being separated by a spacer of 10-15 residues (including 12-13 residues). For example, the protein may include at least 1 and preferably at least 2 EF hand motifs of the form: (D/N)-X1-(D/N)-X2-(D/N)-X3-X4-X5-X6-X7-X8-(E/D), wherein each numbered X is any residue (not necessarily the same residue in each position); X6 and/or X8 is a D or E, and glycine is preferred but not required at X3.

In embodiments, the LanM can include any natural or unnatural amino acid substituted into these EF hands, or elsewhere in the protein. In embodiments, the protein used in the methods of the disclosure can include any number of lanmodulin domains (referred to herein as LanM units) linked together with an appropriate amino acid spacer into a single polypeptide unit. In embodiments, the LanM includes only a single LanM protein unit. In embodiments, the LanM as used herein includes a proline residue at the second position. In embodiments, the LanM as used herein includes 10-15 residues between at least two adjacent EF hands, such as 12-13 residues. In some embodiments, the LanM as used herein includes additional carboxylate residues in one or more of the EF hands, such as, 3 or more, 4 or more, or 5 or more, carboxylate residues.

wild-type M. extorquens LanM SEQ ID NO:1 (EF hand motifs underlined)

APTTTTKVDIAAFDPDKDGTIDLKEALAAGSAAFDKLDPDKDGTLDAKE
LKGRVSEADLKKLDPDNDGTLDKKEYLAAVEAQFKAANPDNDGTIDARE
LASPAGSALVNLIR

Some examples of homologs of the wild type include (note: sequences are shown after removal of the predicted signal sequence):

### Example A: RH AL1

AKMDMKAIDPDSDGTVSLAEAQDAAAKKFAAMDPDNDGTIDLKEAKGK
MAKAKFKKTDADNDGTVDKAEYSALVESAFKAADPDGDGTLDAKELKTP
AGQKLLSLIQ

In this protein, one of the EF hands lacks a proline, and EF1 lacks a carboxylate at positions 9 and 11, but it still undergoes a conformational response at free concentrations of rare earth elements in the picomolar range.

### Example B: Hansschlegelia sp

ASGADALKALNKDNDDSLEIAEVIHAGATTFTAINPDGDTTLESGETKG
RLTEKDWARANKDGDQTLEMDEWLKILRTRFKRADANKDGKLTAAELDS
KAGQGVLVMIMK

In this protein (31% identity), only one of the EF hands has a proline at the second position, and only one has an Asp residue at the first position. However, it still undergoes a conformational response to free concentrations of rare earth elements in the picomolar range and much more weakly to other metals (e.g. calcium).

### Example C: Xanthomonas axonopodis

AQAQVQVQDSQQYLQRMDTDGDGRVSLDEYLAWMSYAFDQRDTDH
DGVLQGDELPG RRGKPITRAAHRATLIARFARQDANGDGYLSAR
ELLAPPR

This protein possesses ˜33% sequence identity with M. extorquens LanM, it contains only 3 EF hands (underlined, referred to as EF1, EF2, and EF4 based on sequence alignment with the M. extorquens LanM), none of which have a proline, and yet it undergoes a conformational response to free concentrations of rare earth elements in the picomolar range and much more weakly to other elements (e.g. calcium). Note that, although EF1 and EF2 are only 13 residues apart, the distance between the adjacent EF2 and EF4 in this sequence is longer (25 amino acids) than in most lanmodulins because one of the EF hands (EF3) is missing.

In certain embodiments, the LanM protein provided herein comprises, consists of, or consists essentially of the amino acid sequence set forth in SEQ ID NO:1 or portion thereof, or a sequence with at least 10%, 15%, 20%, 25%, 30%, 35%, 40%, 45%, 50%, 55%, 60%, 70%, 75%, 80%, 85%, 90%, 91%, 92%, 93%, 94%, 95%, 96%, 97%, 98%, or 99% identity to SEQ ID NO:1.

In certain embodiments, the LanM protein provided herein comprises, consists of, or consists essentially of the amino acid sequence set forth in SEQ ID NO:2 or portion thereof, or a sequence with at least 10%, 15%, 20%, 25%, 30%, 35%, 40%, 45%, 50%, 55%, 60%, 70%, 75%, 80%, 85%, 90%, 91%, 92%, 93%, 94%, 95%, 96%, 97%, 98%, or 99% identity to SEQ ID NO:2.

In certain embodiments, the LanM protein provided herein comprises, consists of, or consists essentially of the amino acid sequence set forth in SEQ ID NO:3 or portion thereof, or a sequence with at least 10%, 15%, 20%, 25%, 30%, 35%, 40%, 45%, 50%, 55%, 60%, 70%, 75%, 80%, 85%, 90%, 91%, 92%, 93%, 94%, 95%, 96%, 97%, 98%, or 99% identity to SEQ ID NO:3.

In certain embodiments, the LanM protein provided herein comprises, consists of, or consists essentially of the amino acid sequence set forth in SEQ ID NO:3 or portion thereof, or a sequence with at least 10%, 15%, 20%, 25%, 30%, 35%, 40%, 45%, 50%, 55%, 60%, 70%, 75%, 80%, 85%, 90%, 91%, 92%, 93%, 94%, 95%, 96%, 97%, 98%, or 99% identity to SEQ ID NO:4.

In certain embodiments wherein the LanM protein comprises an amino acid sequence with less than 100% identity to SEQ ID NO:1, SEQ ID NO:2, SEQ ID NO:3, SEQ ID NO:4, or a portion thereof, all of the substitutions giving rise to the sequence differences are conservative substitutions. In other embodiments, the LanM protein provided herein may comprise one or more non-conservative substitutions versus the LanM protein. In certain embodiments, the LanM protein provided herein comprises one, two, three, four, or five or more conservative substitutions versus a LanM protein.

TABLE 1
Exemplary LanM Amino Acid Sequences

| SEQ | | |
|---|---|---|
| ID NO. | NAME | SEQUENCE |
| 1 | LanM | APTTTTKVDIAAFDPDKDGTIDLKEALAAGSAAFDKLDPDKD |
| | | GTLDAKELKGRVSEADLKKLDPDNDGTLDKKEYLAAVEAQ |
| | | FKAANPDNDGTIDARELASPAGSALVNLIR |
| 2 | LanM-GSGC | APTTTTKVDIAAFDPDKDGTIDLKEALAAGSAAFDKLDPDKD |
| | | GTLDAKELKGRVSEADLKKLDPDNDGTLDKKEYLAAVEAQ |
| | | FKAANPDNDGTIDARELASPAGSALVNLIRGSGC |
| 3 | LanM Δ1-40 | TIDLKEALAAGSAAFDKLDPDKDGTLDAKELKGRVSEA |
| | | DLKKLDPDNDGTLDKKEYLAAVEAQFKAANPDNDGTI |
| | | DARELASPAGSALVNLIRHHHHHH |
| 4 | LanM-double (2-1) | APTTTTKVDIAAFDPDKDGTIDLKEALAAGSAAFDKLD |
| | | PDKDGTLDAKELKGRVSEADLKKLDPDNDGTLDKKEY |
| | | LAAVEAQFKAANPDNDGTIDARELASPAGSALVNLIRG |
| | | SGAEAAAKEAAAKEAAAKEAAAKAAPTTTTKVDIAAFD |
| | | PDKDGTIDLKEALAAGSAAFDKLDPDKDGTLDAKELK |
| | | GRVSEADLKKLDPDNDGTLDKKEYLAAVEAQFKAANP |
| | | DNDGTIDARELASPAGSALVNLIR |

REE are a group of seventeen chemical elements that includes yttrium and fifteen lanthanide elements. Scandium is found in most REE deposits and is often included.

TABLE 2
Rare Earth Elements

| | | Atomic | | | Atomic |
|---|---|---|---|---|---|
| Name | Symbol | Number | Name | Symbol | Number |
| lanthanum | La | 57 | dysprosium | Dy | 66 |
| cerium | Ce | 58 | holmium | Ho | 67 |
| praseodymium | Pr | 59 | erbium | Er | 68 |
| neodymium | Nd | 60 | thulium | Tm | 69 |
| promethium | Pm | 61 | ytterbium | Yb | 70 |
| samarium | Sm | 62 | lutetium | Lu | 71 |
| europium | Eu | 63 | scandium | Sc | 21 |
| gadolinium | Gd | 64 | yttrium | Y | 39 |
| terbium | Tb | 65 | | | |

The REE-binding proteins can bind any of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), scandium (Sc), yttrium (Y), or any combination thereof. The REE binding ligands can bind any of the elements in any oxidation state (e.g., Ln²⁺, Ln³⁺, Ln⁴⁺, etc.)

In some embodiments, the REE binding protein (e.g., LanM) binds a trivalent lanthanide ion (e.g. a REE) with a binding affinity (i.e., K_{d}, the thermodynamic dissociation constant commonly used for protein complexes) of between about 0.1 μM to about 1 μM. In some embodiments, the K_{d} is about 0.1 μM, about 100 μM, about 500 μM, about 1 nM, about 10 nM, about 50 nM, about 100 nM, about 500 nM, or about 1 μM. In still other embodiments, the K_{d} is in the pM range. Affinity can be determined by any suitable means known to one of skill in the art. Non-limiting examples include, titration with REE and detection using fluorescence, circular dichroism, ICP, NMR or calorimetry. In the case of tightly binding sequences, it may be necessary to employ competition experiments.

In some embodiments, the LanM includes a cysteine residue. In some embodiments, the cysteine residue is a C-terminal cysteine residue, an N-terminal cysteine residue, or an internal cysteine residue. In some embodiments, a short, hydrophilic, flexible linker separates the cysteine residue from LanM. Exemplary linkers include those having a glycine-serine amino acid chain comprising one to ten repeats of GlyxSery, wherein x and y are each independently an integer from 0 to 10, provided that x and y are not both 0 (e.g., GlySerGly, (Gly₄Ser)₂; (Gly₃Ser)₂; Gly₂Ser; or a combination thereof, such as (Gly₃Ser)₂Gly₂Ser).

In some embodiments, the LanM protein provided herein comprises, consists of, or consists essentially of the amino acid sequence set forth in SEQ ID NO:1 or portion thereof and a C-terminal cysteine residue, for example, GSGC. In some embodiments, the LanM protein provided herein comprises, consists of, or consists essentially of the amino acid sequence set forth in SEQ ID NO:2 or portion thereof and a C-terminal cysteine residue, for example, GSGC. In some embodiments, the LanM protein provided herein comprises, consists of, or consists essentially of the amino acid sequence set forth in SEQ ID NO:3 or portion thereof and a C-terminal cysteine residue, for example, GSGC.

### Biosorption Systems

Also provided are systems (i.e., biosorption/adsorption media) for REE extraction and preferential separation comprising an amount of the proteins including the LanM protein.

Biosorption is a chemical process based on a variety of mechanisms such as adsorption, absorption, ion exchange, surface complexation, and precipitation. When coupled with a material of biological origin such as proteins or biomass, this material is referred to as biosorption material. A biosorption material can for example, bind to REEs and separate them from REE containing materials (e.g., feedstocks). Provided herein are biosorption materials comprising proteins for preferentially separating REEs from REE containing material. REE extraction and preferential separation comprising an amount of the proteins.

These biosorption media, which include, for example, biofilm, microbeads, and carbon nanotube embedded membranes can be used for adsorption under continuous flow. It is contemplated that protein immobilization in biosorption media for use in flow through setups allows for complete (or substantially complete) separation of REEs from REE-containing mixed metal solutions to include separation of REEs from other REEs in the mixed metal solution in a single step and, for example, without the need for liquid-liquid separation, centrifugation, filtration, or both.

In some embodiments, the biosorption material is a bead and/or capsule. In some embodiments, the bead and/or capsule is suitable for the separation of REEs. In some embodiments, the bead and/or capsule comprises a REE selective protein. In some embodiments, the biosorption material is a microbead. As used herein, the term the term “capsule” is used interchangeably with “bead.”

In some embodiments, the proteins are attached to a solid support, for example, a column, a membrane, a bead, or the like. In some embodiments, the solid support is a porous support material. The solid support can be any suitable composition known to one of skill in the art including, for example, a polymer, agarose, alginate, acrylamide, regenerated cellulose, cellulose ester, plastic, or glass.

In one embodiment, the proteins are bound (i.e., embedded) within or to the surface of a bead. In some embodiments, the bead is a polymer. Suitable polymers include PEG (e.g., ˜10% PEG), alginate (e.g., ˜2% calcium alginate), acrylamide (e.g., ˜10% polyacrylamide), and agarose. In other embodiments the beads are glass, plastic, or steel.

In other embodiments, the disclosure provides methods of preparing a microbead solution for REE separation. In some embodiments, the methods for preparing a bead for REE separation comprise: (a) providing a protein that can selectively bind an REE and a porous support material functionalized with a conjugation agent, and (b) conjugating the protein to the porous support material via the conjugation agent.

In some embodiments, the methods comprise functionalizing the support material with a terminal amine (e.g., —NH₂). In some embodiments, the porous support material comprises agarose. In some embodiments, the porous support material is amine functionalized agarose.

In some embodiments, the conjugation agent is maleimide. In such embodiments, the methods may further comprise reacting the terminal amine with a maleimide to form a maleimide functionalized agarose bead. In some embodiments, the maleimide is succinimidyl 4-(N-maleimidomethyl)cyclohexane-1-carboxylate (SMCC).

In some embodiments, the methods further comprise conjugating a maleimide functionalized porous support material with the REE selective protein. In some embodiments, the protein includes a cysteine residue. In some embodiments, the porous support material is conjugated to the REE selective protein via the maleimide of the porous support material and the cysteine residue of the protein. In some embodiments, the REE selective protein including the cysteine residue is conjugated to the maleimide of the porous support material via “click” chemistry. Conjugating the REE selective protein to the porous support material via “click” chemistry is advantageous as it enables conjugation under biocompatible reaction conditions, namely aqueous conditions devoid of organic solvents.

In some embodiments, conjugating the protein to the porous support material can comprise any bioconjugation methodology using a conjugation agent. In some embodiments, the bioconjugation method uses thiol-ene “click” chemistry. In some embodiments, N-hydroxysuccinimide (NHS) can be used as a conjugation agent for bioconjugation of the protein to the porous support structure. Exemplary NHS crosslinker agents that can also be used as conjugation agents include dibenzycyclooctyne-N-hydroxysuccinimide (DBCO-NHS), bicyclononyne-N-hydroxysuccinimide (BCN-NHS), and dibenzocyclooctyne-sulfo-N-hydroxysuccinimide (DBCO-sulfo-NHS). In some embodiments, the bioconjugation method uses alkyne/azide “click” chemistry. For example, alkyne/azide “click” chemistry can be utilized to conjugate a protein comprising an azide moiety to a porous support material comprising an alkyne moiety used as the conjugation agent.

In some embodiments, the bead has a high concentration of REE-selective proteins. It is contemplated that a high loading of proteins can act, at least in part, to enhance the saturation capacity of the biosorption material by increasing the number of available REE binding sites. An increased number of REE binding sites leads to a larger percentage of REEs from the REE-containing material that complex with the REE binding ligands to form a protein-REE complex (e.g., increased saturation capacity). In some embodiments, the increase in saturation capacity correlates with an increase in adsorption capacity (i.e., an increase in the number of REEs that complex with REE selective proteins per unit volume or unit mass of the REE-containing material). It is contemplated that an increased saturation and adsorption capacity obviates the need from additional and energy-exhaustive steps such as centrifugation and filtration in the process of separating REEs.

In some embodiments, the high concentration of the protein (e.g., LanM) is about 2 μmol of protein per mL of total porous support material (e.g., a microbead) (pmol/mL), 2.2 μmol/mL, 2.4 μmol/mL, 2.6 μmol/mL, 2.8 μmol/mL, 3.0 μmol/mL, 3.2 μmol/mL, 3.4 μmol/mL, 3.6 μmol/mL, 3.8 μmol/mL, 4 μmol/mL, 5 μmol/mL, 10 μmol/mL, 15 μmol/mL, 20 μmol/mL, 25 μmol/mL, 30 μmol/mL, 35 μmol/mL, 40 μmol/mL, 45 μmol/mL, or 50 μmol/mL. In some embodiments, the high concentration of the protein (e.g., LanM) is between about 2 μmol/mL to about 4 μmol/mL, about 3 μmol/mL to about 4 μmol/mL, about 2.2 μmol/mL to about 3 μmol/mL, about 3.2 μmol/mL to about 4 μmol/mL, about 2 μmol/mL to about 3.6 μmol/mL, about 10 μmol/mL to about 30 μmol/mL, about 20 μmol/mL to about 30 μmol/mL, about 10 μmol/mL to about 40 μmol/mL, about 20 μmol/mL to about 40 μmol/mL, or about 25 μmol/mL to about 50 μmol/mL of total porous support material (e.g., a microbead).

In some embodiments, the high concentration of proteins is at least about 20 weight percent (wt %), at least about 5 wt %, at least about 10 wt %, at least about 15 wt %, at least about 20 wt %, at least about 25 wt %, at least about 30 wt %, at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, at least about 80 wt %, at least about 85 wt %, at least about 90 wt %, at least about 95 wt %, or more of the total weight of the bead or at least about 20 volume percent (vol %), at least about 5 vol %, at least about 10 vol %, at least about 15 vol %, at least about 20 vol %, at least about 25 vol %, at least about 30 vol %, at least about 35 vol %, at least about 40 vol %, at least about 45 vol %, at least about 50 vol %, at least about 55 vol %, at least about 60 vol %, at least about 65 vol %, at least about 70 vol %, at least about 75 vol %, at least about 80 vol %, at least about 85 vol %, at least about 90 vol %, at least about 95 vol % or more of the total porous support material (e.g., a microbead).

In some embodiments, the high adsorption capacity of the proteins is at least about 1 milligram (mg), at least about 2 mg, at least about 3 mg, at least about 4 mg, at least about 5 mg, at least about 6 mg, at least about 7 mg, at least about 8 mg, at least about 9 mg, at least about 10 mg, at least about 11 mg, at least about 12 mg, at least about 13 mg, at least about 14 mg, at least about 15 mg, at least about 16 mg, at least about 17 mg, at least about 18 mg, at least about 19 mg, at least about 20 mg, at least about 21 mg, at least about 22 mg, at least about 23 mg, at least about 24 mg, at least about 25 mg, at least about 26 mg, at least about 27 mg, at least about 28 mg, at least about 29 mg, at least about 30 mg, at least about 31 mg, at least about 32 mg, at least about 34 mg, at least about 35 mg, at least about 36 mg, at least about 37 mg, at least about 38 mg, at least about 39 mg, at least about 40 mg, at least about 41 mg, at least about 42 mg, at least about 43 mg, at least about 44 mg, at least about 45 mg, at least about 46 mg, at least about 47 mg, at least about 48 mg, at least about 49 mg, or at least about 50 mg of REE per gram (g) of protein.

In some embodiments, the high adsorption capacity of the protein is at least about 1 milligram (mg), at least about 2 mg, at least about 5 mg, at least about 10 mg, at least about 15 mg, at least about 20 mg, at least about 25 mg, at least about 30 mg, at least about 35 mg, at least about 40 mg, at least about 45 mg, at least about 50 mg, at least about 60 mg, at least about 65 mg, at least about 70 mg, at least about 75 mg, at least about 80 mg, at least about 85 mg, at least about 90 mg, at least about 95 mg, or at least about 100 mg of REE per gram (g) of proteins. In some embodiments, the proteins have an adsorption capacity between about 30 to about 70 mg of REE per g of protein.

In some embodiments, the high adsorption capacity of the protein is at least about 0.1 milligram (mg), at least about 2 mg, at least about 5 mg, at least about 10 mg, at least about 15 mg, at least about 20 mg, at least about 25 mg, at least about 30 mg, at least about 35 mg, at least about 40 mg, at least about 45 mg, at least about 50 mg, at least about 60 mg, at least about 65 mg, at least about 70 mg, at least about 75 mg, at least about 80 mg, at least about 85 mg, at least about 90 mg, at least about 95 mg, at least about 100 mg of REE per gram (g) of biosorption media (e.g., system and/or material). In some embodiments, the proteins have an adsorption capacity between about 30 to about 70 mg of REE per g of biosorption media.

In another embodiment, the proteins are encapsulated within and/or on a surface of the bead. When the proteins are encapsulated within and/or on the surface of the bead, the beads are able to efficiently bind the REEs. Once the REE-containing material is flowed on and/or through the bead, the immobilized LanM proteins are able to capture the REEs both within and on the surface of the bead, which optimizes the adsorption capacity of the bead by increasing the ratio of available binding sites (i.e., binding ligands) to total volume of the bead.

In some embodiments, the support material (e.g., microbeads) is porous. In some embodiments, the support material is a fibrous material. For example, in some embodiments, the support material is a fibrous material produced with electrospun fibers. The porous support material enables the flow of the REE containing material to contact not only the exterior surface, but also, the interior surface of the bead thereby increase the saturation and adsorption capacity of the support material for the REEs (i.e., increased accessibility). In some embodiments, the porous support materials have a pore diameter of at least about 0.10 nm, at least about 1.0 nm, at least about 10 nm, at least about 50 nm, at least about 100 nm, at least about 150 nm, at least about 200 nm, at least about 250 nm, at least about 300 nm, at least about 350 nm, at least about 400 nm, at least about 450 nm, at least about 500 nm, at least about 550 nm, at least about 600 nm, at least about 650 nm, at least about 700 nm, at least about 750 nm, at least about 800 nm, at least about 850 nm, at least about 900 nm, at least about 950 nm, or at least about 1000 nm. In some embodiments, the porous support materials have a pore diameter between about 1.0 nm to about 500 nm, about 0.10 nm to about 10 nm, about 150 nm to about 1000 nm, about 300 nm to about 600 nm, about 200 nm to about 800 nm, about 300 nm to about 500 nm, about 500 nm to about 1000 nm, or about 600 nm to about 800 nm.

### Methods

Also provided are methods of using proteins, for example LanM to preferentially separate REEs from REE-containing materials, and to separate REEs from other REEs.

Preferential separation of REEs from REE-containing materials is crucial for the development of technologies such as batteries, magnets, and electronics, however the similarity in the chemical properties of REEs makes them exceedingly challenging to separate from each other as well as from other non-REEs. Prevailing technologies are hampered by low selectivity towards REEs, particularly when the REEs are in the presence of a mixture of competing metals such as non-REEs (e.g., alkali, alkaline, and other transition metals). Even more challenging, is the separation of REEs from the competing metals wherein the competing metals are present at much higher concentration than the REEs. Due to the innate challenges in separating REEs, conventional technologies are limited in their ability to separate REEs in high purity and further has prevented the development of systems capable of preferentially separating REEs on an industrial, bulk scale. The present disclosure provides methods for the preferential separation of REEs with a high selectivity, high efficiency, and at a low cost, characteristics that each lend themselves towards a system capable of separating REEs on a large, industrial scale. In some embodiments, the methods for preferentially separating REEs from a REE-containing material include separating individual REEs or groups of REEs from other REEs or non-REEs. Preferential separation an individual REE refers to the isolation of an REE (e.g., La) from either another REE (e.g., Er), group of REEs, or a non-REE, wherein there is no or substantially no other element, REE or non-REE, present after the separation (i.e., in the eluted solution). For example, preferential separation methods provided herein enable the separation of Nd (i.e., an individual REE) from Y, Dy, and Tb (i.e., groups of REEs) and/or from competing metals (i.e., non-REEs). In some embodiments, the preferential separation provides high purity separation of individual REEs, wherein there is no or substantially no other individual REE, group of REEs and/or non-REEs present after the separation (i.e., in the eluted solution).

In one aspect provided herein are methods for preferentially separating REEs from a REE-containing material comprising the steps of: (a) providing a protein that can selectively bind one or more REEs; (b) contacting the protein with the REE-containing material, wherein the protein binds at least a portion of the one or more REEs to form a protein-REE complex and an REE-depleted material; (c) separating the protein-REE complex from at least a portion of the REE-depleted material; and (d) separating the REE from the protein to produce a regenerated protein. In some embodiments, the steps described are executed once. In other embodiments, the steps or a portion of the steps are executed more than once, for example, 2, 3, 4, 5, or more times. In some embodiments, the steps or portions of the steps are executed more than once with more than one REE-containing material, for example with 1, 2, 3, 4, 5, or more REE-containing materials.

In some embodiments, the steps or portions of the steps are repeated until at least about 100%, at least about 90%, at least about 80%, at least about 70%, at least about 60%, at least about 50%, at least about 40%, at least about 30%, at least about 20%, or at least about 10% of the REEs are separated from the protein-REE complex.

In some embodiments, the proteins are added to a column prior to contacting the proteins with the REE-containing material. In some embodiments, prior to adding the proteins to the column, the proteins are conjugated within or to the surface of a solid structure (e.g., a bead and/or capsule). When added to the column, the proteins are used, as defined conventionally in column chromatography, as the stationary phase. This enables a continuous flow system in which REE containing material is introduce to the column, and flows through the column. In some embodiments, the flow is from the top to the bottom of the column. In some embodiments, the flow is reversed and the flow is from the bottom to the top of the column.

In certain embodiments, a method for separating rare earth elements (REE) from a REE-containing material comprising one or more adsorption/desorption cycles. In some embodiments, the methods disclosed herein may include two or more adsorption/desorption cycles. In some embodiments, each cycle includes steps where the REEs are adsorbed to and desorbed from the protein separately. For example, in some embodiments, upon contacting the proteins with the REE-containing material, the REEs are adsorbed to the protein (i.e., form a protein-REE complex) and then upon introducing the solution, the REEs are desorbed from the REE binding ligand (i.e., break apart the protein-REE complex by dissociating the REE from the protein). In some embodiments, the REE-containing material is flowed over a column comprising the proteins until the column is saturated (i.e., all or substantially all protein binding sites are bound to an REE to form a protein-REE). In some embodiments, at least about 95%, at least about 90%, at least about 85%, at least about 80%, at least about 75%, at least about 70%, at least about 65%, at least about 60%, at least about 55%, or at least about 50% of the protein binding sites are bond to an REE. The initial adsorption step results in the majority (i.e., greater than 50%) of the non-REEs flowing through the column while the REEs are retained (i.e., bound by the REE binding ligand). The column is then washed with a solution to wash out or separate the residual unbound REEs or non-REEs from the protein-REE complexes. After saturation and wash, the solution is used to differentially separate (i.e., elute) the REEs from the proteins. In accordance with the embodiments disclosed herein, the differentially separated/eluted REE solution(s) are REE-enriched solutions that can have a higher concentration of REE as compared to the feed solution.

In some embodiments, the REEs are adsorbed and desorbed from the REE binding ligand simultaneously during mass transport process. For example, in some embodiments, the REE-containing material is flowed over a column comprising the proteins, wherein only a portion of the protein binding sites adsorb an REE to from a protein-REE complex. A solution is then flowed through the column, wherein the REEs undergo a series of adsorption and desorption (i.e., is a dynamic process) from the REE binding ligand as REEs proceed from the top to the bottom of the column. The difference in affinity of each REE for the REE binding ligand (i.e., solid phase) and the tunable solution (i.e., mobile phase) controls the migration rate of the REEs through the column. Heavier REEs migrate faster than lighter REEs through the column due to the weaker complexes that are formed between the heavier REEs and the protein as compared to the lighter REEs and the protein, providing a method for the preferential separation of the REEs. Specifically, Y migrates with the HREEs, whereas Sc migrates the slowest through column given that Lanmodulin forms the strongest complex with Sc. The simultaneous adsorption and desorption process enables the separation of individual and/or groups of REEs in a high purity. This method provides advantages over conventional REE separation process which require the use of expensive chemical resins or with hazardous solvents in order to obtain highly purified REEs and/or group of REEs.

In some embodiments, the methods for preferentially separating REEs from REE-containing material further comprise introducing a modifiable solution. The modifiable solution is a solution with a different concentration of chelator and/or different pH compared to the initial solution used to introduce REEs to the protein. The modifiable solution preferentially separates REEs from the REE-containing material. The preferential separation can be achieved when the solution is modified in a manner in which the affinity of an individual or group of REEs for the solution (i.e., mobile phase) is increased while the affinity of the individual or groups of REEs for the REE binding ligands (i.e., stationary phase) is decreased. Conversely, the preferential separation can also be achieved when the solution is modified in a manner in which the affinity of an individual or groups of REEs for the REE binding ligand (i.e., stationary phase) is increased while the affinity of the individual or group of REEs for the REE solution is decreased.

In some embodiments, the REEs are adsorbed to and desorbed from the protein separately in order to selective desorb one or more REEs from other REEs. For example, in some embodiments, the REE-containing material is flowed over a column comprising the proteins until the column is saturated (i.e., all or substantially all protein binding sites are bound to an REE to form a protein-REE). In some embodiments, at least about 95%, at least about 90%, at least about 85%, at least about 80%, at least about 75%, at least about 70%, at least about 65%, at least about 60%, at least about 55%, or at least about 50% of the protein binding sites are bond to an REE. In some embodiments, once saturated, one or more solutions are used to differentially separate (i.e., elute) one or more REEs from the proteins in a sequentially manner. For example, in some embodiments, a first solution is used to separate HREEs, a second solution used to separate MREEs, and a third solution is used to separate LREEs. In other embodiments, one or more solutions are used to preferentially separate one or more REEs from the proteins.

In some embodiments, the methods for preferentially separating REEs are continuous and the REE separation is uninterrupted by additional energy-intensive steps such as centrifugation and/or filtration. In other embodiments, the methods for preferentially separating REEs comprise an additional step of centrifugation filtration, or both.

In some embodiments, a protein-REE complex is formed at a pH of about 2.4 to about 7 In some embodiments, a protein-REE complex is formed at a pH of about 2.4, about 2.5, about 2.6, about 2.7, about 2.8, about 2.9, about 3, about 3.5, about 4, about 4.5, about 5, about 5.5, about 6, about 6.5, about 7, about 7.5, about 8, about 8.5, about 9, about 9.5, or about 10. In some embodiments, a protein-REE complex is formed at a pH of about 2.4 to about 10, wherein the protein does not bind any non-REE component (e.g., non-REE metals).

In some embodiments, an REE-selective protein binds two REEs per protein. For example, in some embodiments, the stochiometric ratio of REE to protein is 2:1. In some embodiments, the protein-REE complex comprises two REEs per one protein. In some embodiments, the protein is LanM and the stochiometric ratio of REE to LanM is 2:1.

In some embodiments, an REE-selective protein binds three REEs per protein. For example, in some embodiments, the stochiometric ratio of REE to protein is 3:1. In some embodiments, the protein-REE complex comprises three REEs per one protein. In some embodiments, the protein is LanM and the stochiometric ratio of REE to LanM is 3:1.

In some embodiments, an REE-selective protein binds four REEs per protein. For example, in some embodiments, the stochiometric ratio of REE to protein is 4:1. In some embodiments, the protein-REE complex comprises four REEs per one protein. In some embodiments, the protein is LanM and the stochiometric ratio of REE to LanM is 4:1.

In some embodiments, an REE-selective protein binds five REEs per protein. For example, in some embodiments, the stochiometric ratio of REE to protein is 5:1. In some embodiments, the protein-REE complex comprises five REEs per one protein. In some embodiments, the protein is LanM and the stochiometric ratio of REE to LanM is 5:1.

In some embodiments, an REE-selective protein binds six REEs per protein. For example, in some embodiments, the stochiometric ratio of REE to protein is 6:1. In some embodiments, the protein-REE complex comprises six REEs per one protein. In some embodiments, the protein is LanM and the stochiometric ratio of REE to LanM is 6:1.

In some embodiments, an REE-selective protein binds seven REEs per protein. For example, in some embodiments, the stochiometric ratio of REE to protein is 7:1. In some embodiments, the protein-REE complex comprises seven REEs per one protein. In some embodiments, the protein is LanM and the stochiometric ratio of REE to LanM is 7:1.

In some embodiments, an REE-selective protein binds eight REEs per protein. For example, in some embodiments, the stochiometric ratio of REE to protein is 8:1. In some embodiments, the protein-REE complex comprises eight REEs per one protein. In some embodiments, the protein is LanM and the stochiometric ratio of REE to LanM is 8:1.

In some embodiments, an REE-selective protein binds nine REEs per protein. For example, in some embodiments, the stochiometric ratio of REE to protein is 9:1. In some embodiments, the protein-REE complex comprises nine REEs per one protein. In some embodiments, the protein is LanM and the stochiometric ratio of REE to LanM is 9:1.

In some embodiments, an REE-selective protein binds ten REEs per protein. For example, in some embodiments, the stochiometric ratio of REE to protein is 10:1. In some embodiments, the protein-REE complex comprises ten REEs per one protein. In some embodiments, the protein is LanM and the stochiometric ratio of REE to LanM is 10:1.

In some embodiments, an REE selective protein is a LanM protein and LanM protein comprises, or consists essentially of the amino acid sequence set forth in SEQ ID NO:4 or portion thereof and binds four REEs per protein. For example, in some embodiments, the stochiometric ratio of REE to protein is 4:1, 5:1, 6:1, 7:1, 8:1, 9:1, or 10:1. In some embodiments, the protein-REE complex comprises four REEs per one protein. In some embodiments, the protein is LanM (SEQ ID NO:4) and the stochiometric ratio of REE to LanM is 4:1, 5:1, 6:1, 7:1, 8:1, 9:1, or 10:1.

In some embodiments, a REE is separated from the protein by contacting the protein-REE complex with a solution having a pH of less than about 2.4 In some embodiments, the solution has a pH of less than about 2.4, less than about 2.3, less than about 2.2, less than about 2.1, less than about 2, less than about 1.9, less than about 1.8, less than about 1.7, less than about 1.6, less than about 1.5, or less. In accordance with the embodiments described herein, a solution having a specific or specified pH is generated by using any solution or composition known in the art to produce that pH, e.g., an HCL solution, an H2SO4 solution, an HNO3 solution, a solution having a mixture of HCl/NaCl, H2SO4/KHSO4/Na2SO4, or other mixture, a solution of glycine, or any other compatible solvent capable of producing the desired solution.

In some embodiments, a REE is separated from the protein by contacting the REE-complex with a solution comprising a chelator. A chelating agent can include any compound comprising a functional group capable of binding non-REE metal or a REE. For example, in certain embodiments, the chelator or chelating agent can be a mono-carboxylic acid, a di-carboxylic acid, or a tri-carboxylic acid. Non-limiting examples of chelators or chelating agents include EDTA, citrate, dimercaprol, malonate, iminodiacetate, diglycolic acid, hydroxyisobutyric acid, polyaminocarboxylates, hydroxypyridinones, catechols, hydroxamates, acetate, nitrilotriacetate, dipicolinic acid, or a-hydroxyisobutyric acid.

In some embodiments, the REEs and/or groups of REEs are separated in a purity of at least about 10%, at least about 15%, at least about 20%, at least about 30%, at least about 40%, at least about 50%, at least about 55%, at least about 60%, at least about 65%, at least about 70%, at least about 80%, at least about 85%, at least about 90%, at least about 95%, or at least about 100%, relative to any other REE and/or group of REEs.

In some embodiments, the REEs and/or groups of REEs are separated in a purity of at least about 10%, at least about 15%, at least about 20%, at least about 30%, at least about 40%, at least about 50%, at least about 55%, at least about 60%, at least about 65%, at least about 70%, at least about 80%, at least about 85%, at least about 90%, at least about 95%, or at least about 100%, relative to any other element.

In some embodiments, the REEs and/or groups of REEs are separated in a purity of at least about 10%, at least about 15%, at least about 20%, at least about 30%, at least about 40%, at least about 50%, at least about 55%, at least about 60%, at least about 65%, at least about 70%, at least about 80%, at least about 85%, at least about 90%, at least about 95%, or at least about 100%, relative to any non-REE component. In some embodiments, the non-REE component is a non-REE element selected from Mg²⁺, Al³⁺, Ca2+, C0²⁺, Ni²⁺, Cu²⁺, Fe²⁺, Fe³⁺, Zn²⁺, U, Th, and other alkali, alkaline earth, and transition metals found in REE-containing feedstocks.

In some embodiments, the methods provided herein allow for the preferential separation of REEs from Fe³⁺. In some embodiments, LanM has a higher selectivity (i.e., binding affinity) to REEs relative to Fe³⁺. In some embodiments, Fe³⁺ is preferentially separated from the REEs by contacting the protein-REE complex with a solution having a pH of about 4 to about 5, wherein Fe³⁺ precipitates from the solution and the REE remains bond to the protein. In some embodiments, the REEs are then separated from the protein-REE complex by contacting the protein-REE complex with a solution having a pH of 2.4 or less.

In some embodiments, the REEs and/or groups of REEs are separated in a purity of at least about 10%, at least about 15%, at least about 20%, at least about 30%, at least about 40%, at least about 50%, at least about 55%, at least about 60%, at least about 65%, at least about 70%, at least about 80%, at least about 85%, at least about 90%, at least about 95%, or at least about 100%, relative to Fe³⁺

In some embodiments, the methods provide preferential separation of REEs adjacent to each other on the periodic table. For example, in some embodiments, the methods provide preferential separation of La from Ce, Ce from Pr, Pr from Nd, Nd from Pm, Pm from Sm, Sm from Eu, Eu from Gd, Gb from Tb, Tb from Dy, Dy from Ho, Ho from Er, Er from Tm, Tm from Yb, and/or Yb from Lu.

In some embodiments, the methods provide preferential separation of REEs from other REEs or non-REE metals having similar ionic radii. For example, in some embodiments, the methods provide preferential separation of La from Y, Ce from Y, Pr from Y, Nd from Y, Pm from Y, Sm from Y, Eu from Y, Gb from Y, Tb from Y, Dy from Y, Ho from Y, Er from Y, Tm from Y, Yb from Y, Lu from Y, or Sc to Y.

In some embodiments, the methods provide preferential separation of REEs from Sc. For example, in some embodiments, the methods provide preferential separation of La from Sc, Ce from Sc, Pr from Sc, Nd from Sc, Pm from Sc, Sm from Sc, Eu from Sc, Gb from Sc, Tb from Sc, Dy from Sc, Ho from Sc, Er from Sc, Tm from Sc, Yb from Sc, Lu from Sc, or Y from Sc.

In some embodiments, the methods provide preferential separation of REEs based on the ionic radius, atomic radius, and/or weight of the REE. In some embodiments, REEs with a smaller ionic radius are preferentially separated from REEs with a large atomic radius. In some embodiments, the preferential separation of the REEs is influenced by the effects of the lanthanide contraction, wherein the ionic radii of the lanthanides significantly decrease upon moving from left to right on the periodic (i.e., La to Lu). In some embodiments, the methods provide preferential separation of REEs based on the ionic radius, atomic radius, and/or weight of the REE using a two-step desorption process.

In some embodiments, the two-step desorption process comprises contacting the protein-REE complex with a first solution having a specific pH and a second solution having a specific pH. In some embodiments, the first step of the desorption process comprises introducing a solution having a first pH to the protein-REE complex and the second step of the desorption process comprises introducing a solution having a second pH to the protein-REE complex. In other embodiments, the desorption process comprises more than two steps. In some embodiments, the desorption process includes a first step of introducing a first solution comprising a specific pH to the protein-REE complex, a second step of introducing a second solution comprising a specific pH, and a third step of introducing a third solution comprising a specific pH. In some embodiments, the desorption process includes a first step of introducing a first solution comprising a specific pH to the protein-REE complex, a second step of introducing a second solution comprising a specific pH, and a third step of introducing a third solution comprising a specific pH, and a fourth step of introducing a fourth solution having a specific pH. In some embodiments, the first pH is about 1.9 to about 2.4. For example, in some embodiments, the first pH is about 1.9, about 2.0, about 2.1, about 2.2, about 2.3, or about 2.4. In some embodiments, the second pH is about 1.5 to about 1.7. For example, in some embodiments, the second pH is about 1.5, about 1.6, or about 1.7. In other embodiments that include more than two steps, the pH is sequentially lowered from between about 2.4 to about 1.5 or less. In some embodiments, contacting the solution having a first pH (i.e., higher pH) with the protein-REE complex separates a REE having a smaller iconic radius from the REE-complex whereas a REE having a larger ionic radius does not separate from the REE-complex. In some embodiments, contacting the solution having a second pH (i.e., lower pH) separates a REE having a smaller ionic radius from the REE complex. In some embodiments, the REE having a smaller ionic radius is Dy or Y and the REE have a greater ionic radius is Nd or Pr. In some embodiments, the methods enable the preferential desorption of Dy from Nd, Dy from Pr, or Y from Nd. In some embodiments, the methods enable to preferential separation of LREEs (e.g., La—Nd), HREEs (Ho—Lu plus Y), and medium REEs (MREEs; Sm—Dy).

In some embodiments, the two-step desorption process comprises contacting the protein-REE complex with a first solution comprising a chelator and another solution (e.g., a second solution) having a specific pH. In some embodiments, the first step of the desorption process comprises introducing a solution comprising a chelator to the protein-REE complex and the second step of the desorption process comprises introducing a solution having a specific pH to the protein-REE complex. In other embodiments, the desorption process comprises more than two steps. In some embodiments, the desorption process includes a first step of introducing a first solution comprising a chelator to the protein-REE complex, a second step of introducing a second solution comprising a chelator solution, and a third step of introducing a third solution having a specific pH. In some embodiments, the desorption process includes a first step of introducing a first solution comprising a chelator to the protein-REE complex, a second step of introducing a second solution comprising a chelator solution, and a third step of introducing a third solution comprising a chelator solution, and a fourth step of introducing a fourth solution having a specific pH. In some embodiments, the chelator is citrate or malonate. In some embodiments, the specific pH is less than 3.0. In some embodiments, the specific pH is less than 2.0. In some embodiments, the specific pH is between about 1.7 to about 2.4. For example, in some embodiments, the pH is about 1.5, about 1.6, about 1.7, about 1.8, about 1.9, about 2.0, about 2.1, about 2.2, about 2.3, or about 2.4. In some embodiments, contacting the solution comprising a chelator with the protein-REE complex separates a REE having a smaller ionic radius from the REE-complex whereas a REE having a larger ionic radius does not separate from the REE-complex. In some embodiments, contacting the solution at a specific pH (e.g., 1.7 to 2.2) separates a REE having a smaller ionic radius from the REE complex. In some embodiments, the REE having a smaller ionic radius is Dy or Y and the REE have a larger ionic radius is Nd or Pr. In some embodiments, the methods enable the preferential desorption of Dy from Nd, Dy from Pr, or Y from Nd. In some embodiments, the methods enable to preferential separation of LREEs (e.g., La—Nd), HREEs (Ho—Lu plus Y), and MREEs (Sm—Dy).

In some embodiments, LREEs such as La, Ce, Pr, Nd, Sm, or Eu are preferentially separated from HREEs such as Gd, Tb, Dy, Ho, Er, Tm, Yb, Lu, Sm, Y, or Sc. In some embodiments, the methods provide preferential separation of LREEs from HREEs comprising the steps of (a) contacting the protein-REE complexes with a chelating agent (e.g., citrate) at a first concentration, wherein the HREEs separate from the protein-REE complexes and (b) contacting the remaining protein-REE complexes (e.g., proteins bound to LREEs) with a chelating agent (e.g., citrate) at a second concentration to separate the remaining REEs. In some embodiments, the first concentration of the chelating agent is about 5 mM to about 10 mM. For example, in some embodiments, the first concentration of the chelating agent is about 5 mM, about 6 mM, about 7 mM, about 8 mM, about 9 mM, or about 10 mM. In some embodiments, the second concentration of the chelating agent is about 15 mM to about 50 mM. For example, in some embodiments, the second concentration of the chelating agent is about 15 mM, about 20 mM, about 25 mM, about 30 mM, about 35 mM, about 40 mM, about 45 mM, or about 50 mM.

In some embodiments, the present disclosure provides methods for preferentially separating REEs in a single step. Single-step separation occurs when the REE-containing material is introduced to the proteins once and results in the isolation and purification of the individual REE or groups of REEs with no or no or substantially no other element from a different group of REEs and/or non-REEs after the separation (i.e., the REE-containing material does not need to be introduced to the column more than once to achieve high purity). This contrasts with conventional REE separation technologies that require multiple rounds of purification of the REE-containing material, which is not only inefficient, but also, expensive.

Aspects of the present disclosure also provide methods of preferentially separating Sc from REE-containing materials using REE selective proteins such as LanM. Sc can be preferentially separated from REEs and non-REE containing material by selectively desorbing Sc from a protein-REE complex (e.g., protein-Sc complex). In some embodiments, Sc is selectively desorbed from the protein by contacting a protein-REE complex with a solution comprising a chelator that separates Sc, but not other REEs from the protein. In some embodiments, the solution comprises malonate. In some embodiments, contacting a protein-REE complex with a solution comprising malonate selectively desorbs Sc without disrupting (e.g., separating REE from the protein) the protein-REE complexes, wherein the REE is a HREE. In a non-limiting embodiment, such a solution (e.g., a first solution) includes malonate at a concentration of 20-50 mM. In another non-limiting embodiment, such a solution (e.g., a first solution) includes citrate at a concentration of about 3.0 mM. Once Sc is desorbed with the first solution, one or more additional solutions can be used to selectively desorb additional REEs. For example, in some embodiments, a second solution containing citrate at a concentration of about 15.0 mM and a third solution containing citrate at a concentration of about 25 mM to about 50 mM, and a fourth solution having a pH of about 1.5 can be sequentially flowed over the protein-REE complexes to remove additional REEs of Y,HREE/MREE, and La/Ce, respectively.

In some embodiments, methods of preferentially separating Scfrom REE-containing materials using REE selective proteins comprises: (a) providing a plurality protein that can selectively bind one or more REEs; (b) contacting the plurality of proteins with the REE containing material, wherein the plurality of proteins bind at least a portion of the one or more REEs to form a plurality of protein-REE complexes and an REE-depleted material; (c) separating the plurality of protein-REE complexes from at least a portion of the REE-depleted material; (d) separating Sc from the plurality of proteins by contacting the plurality of protein-REE complexes with a solution comprising a chelator, for example, malonate or citrate; (e) separating HREEs (e.g., Lu, Yb) from the plurality of proteins by contacting the plurality of protein-REE complexes with a solution comprising a low concentration of a chelator; (f) separating Y from the plurality of proteins by contacting the plurality of protein-REE complexes with a solution comprising an intermediate concentration of a chelator (e.g., malonate or citrate) or a solution comprising a pH of about 2.3; (g) separating MREEs from the plurality of proteins by contacting the plurality of protein-REE complexes with a solution comprising a high concentration of a chelator or a solution having an intermediate pH; and (h) separating LREEs from the plurality of proteins by contacting the plurality of protein-REE complexes with a solution comprising a low pH (1.7). In some embodiments, the chelator is citrate. In some embodiments, a solution comprising a low concentration of a chelator comprises the chelator at a concentration of about 5 mM. In some embodiments, the chelator is citrate. In some embodiments, a solution comprising an intermediate concentration of a chelator comprises the chelator at a concentration of about 15 mM. In some embodiments, a solution comprising a high concentration of a chelator comprises the chelator at a concentration of about 30 mM. In some embodiments, a solution having a higher pH has a pH of about 2.3. In some embodiments, a solution having an intermediate pH has a pH of about 2.1 In some embodiments, a solution having a low pH has a pH of about 1.7.

The REE-containing material may be any material known to contain or suspected to contain REE. In some embodiments the material is a solid material, a semi-solid material, or an aqueous medium. In a preferred embodiment, the material is an aqueous solution. Non-limiting examples of suitable materials for use in extraction of REE include leachates derived from rare earth ores (e.g., bastnasite, monazite, loparite, xenotime, allanite, and the lateritic ion-adsorption clays), geothermal brines, coal, coal byproducts, mine tailings, phosphogypsum, acid leachate of solid source materials, REE solution extracted from solid materials through ion-exchange methods, or other ore materials, such as REE-containing clays, volcanic ash, organic materials, and any solids/liquids that react with igneous and sedimentary rocks.

In some embodiments, the REE-containing material is a low-grade material wherein the REEs are present in less than about 2 wt % of the total weight of the low-grade material. In other embodiments, the REE-containing material is a high-grade material, wherein the REE are present in greater than about 2 wt % of the total weight of the high-grade material.

In some embodiments, the REE-containing material comprises less than about 5 wt %, less than about 10 wt %, less than about 15 wt %, less than about 20 wt %, less than about 25 wt %, less than about 30 wt %, less than about 35 wt %, less than about 40 wt %, less than about 45 wt %, less than about 50 wt % REEs of the total weight of the REE-containing material.

The proteins can also be used for recovering REE from recycled REE-containing products such as, compact fluorescent light bulbs, electro ceramics, fuel cell electrodes, NiMH batteries, permanent magnets, catalytic converters, camera and telescope lenses, carbon lighting applications, computer hard drives, wind turbines, hybrid cars, x-ray and magnetic image systems, television screens, computer screens, fluid cracking catalysts, phosphor-powder from recycled lamps, and the like. These materials are characterized as containing amounts of REE, including, for example, scandium, yttrium, lanthanum, cerium, praseodymium, neodymium, samarium, promethium, europium, gadolinium, terbium, dysprosium, erbium, thulium, ytterbium, lutetium, or any combination thereof. In some embodiments, the REEs are recovered from a liquid waste stream from a given industry (e.g., an effluent from a factory that needs to be decontaminated from its REEs or hospital effluents for potential recovery of gadolinium) or a leachate solution coming from these REE containing materials.

In some embodiments, the material is pre-processed prior to providing the proteins. Non-limiting examples of suitable pre-processing includes acid leaching, bioleaching, ion-exchange extraction, pH adjustment, iron oxide precipitation, temperature cooling (e.g., geothermal brines). In other embodiments, prior to providing the proteins, the REE-containing material is refined to remove at least a portion of non-REE metals.

In some embodiments, at least a portion of the proteins are attached (i.e., immobilized) to a surface of a solid support prior to contacting with a REE-containing material. In some embodiments, attachment of the protein to the surface of a solid support is reversible. It is contemplated that protein immobilization in biosorption medium for use in flow-through setups allows for complete (or substantially complete) separation of REEs from REE-containing mixed metal solutions in a single step. In one embodiment, about 20%, about 30%, about 40%, about 50%, about 60%, about 70%, about 80%, about 90%, about 91%, about 95%, about 97%, about 98%, about 99%, or 100% of the REE in the REE-containing material (e.g., mixed metal solution) is extracted in a single step. In some embodiments, about 1%, 5%, 10%, 15%, 20%, about 30%, about 40%, about 50%, about 60%, about 70%, about 80%, about 90%, about 91%, about 95%, about 97%, about 98%, about 99%, or 100% more of the REE in the REE-containing material (e.g., mixed metal solution) is extracted in a single step as compared to an amount of REE extracted in a single step using conventional extraction methods.

The binding of REE to the proteins can be reversible. In some embodiments, at least a portion of the REE in the proteins-REE complex is desorbed (i.e., removed or separated) from the proteins. Non-limiting examples of suitable methods include acid treatment (e.g., sulfuric acid/HNO3 and HCl), citrate, acetate, malonate, and gluconate. In a preferred embodiment, the removal step is performed by acid-stripping. In another preferred embodiment, wherein the removal step is performed using an amount of citrate.

The proteins can also be reused. In some embodiments, the methods further comprise removing the REE from the proteins to regenerate proteins. The proteins can be used 2, 3, 4, 5, 6, 7, 8, 9, 10, 15, 20, 25, 30, or more times. In other embodiments, the proteins are single use. The proteins can be re-conditioned by any means known to one of skill in the art. For example, the proteins may be cleaned with buffer to wash off the citrate to re-generate proteins. In one embodiment, the methods further comprise reusing the regenerated proteins to carry out the extraction of REE from REE-containing material.

The proteins can be reused 2, 3, 4, 5, 6, 7, 8, 9, 10, 15, 20, 25, 30 or more times while also maintaining their high adsorption capacity. In some embodiments, the proteins maintain an adsorption capacity of about 10 mg of REE, about 15 mg of REE, about 20 mg of REE, about 30 mg of REE, about 40 mg of REE, about 50 mg of REE, about 60 mg of REE, or about 70 mg of REE per g of the proteins during each of the adsorption cycles. In some embodiments, the proteins maintain an adsorption capacity of about 30 to about 70 mg of REE per g of the proteins for 9 cycles.

Aspects of the disclosure provide a kit of parts comprising: (a) a protein that can selectively bind an REE and (b) instructions for differentially separating REEs from a REE-containing material. In some embodiments, the protein is conjugated to a porous support material (e.g., microbead).

### EXAMPLES

### Example 1: Protein-Based Method for REE Separation

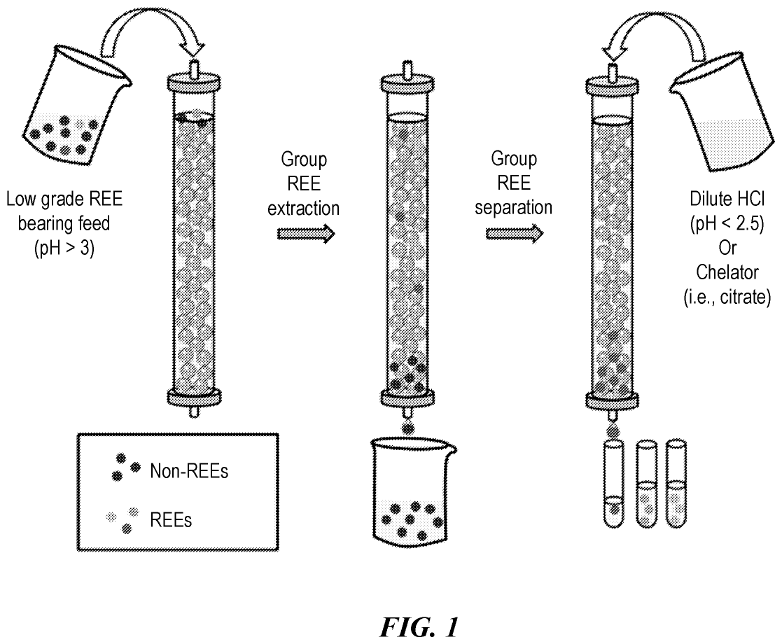

The extraction and subsequent separation of individual REEs starting from REE-bearing feedstocks represents a challenging yet essential task for the growth and sustainability of renewable energy technologies. As an important step toward overcoming the technical and environmental limitations of current REE processing methods, the following example demonstrates a bio-based, all-aqueous REE extraction and separation scheme using the REE-selective lanmodulin (LanM) protein. LanM was conjugated onto porous support materials using thiol-maleimide click chemistry to enable REE purification and separation under flow-through conditions. Immobilized LanM maintains the attractive properties of the free protein, including remarkable REE selectivity and the ability to bind REEs at low pH. This example further demonstrates the ability of LanM to achieve high-purity separation of the clean energy critical REE pairs Nd/Dy and Nd/Y and to transform an industrially relevant low-grade leachate into separate heavy and light REEs fractions in a single column run. A key advantage of the following process over the prior art is the compatibility with low-grade feedstock leachates, the lack of organic solvents, and the ability to achieve efficient separation of certain REE pairs while using ˜90% of the column capacity, which contrasts with traditional ion exchange chromatography. This technology may be further developed to enable a sustainable, low-cost REE recovery and separation process that is broadly applicable to REE feedstocks. FIG. 1 provides a simplified schematic depicting the overall process of REE separation outlined in Example 1.

### Methods

### Chemicals and Materials

REE chloride salts (>99.9%), solvents, and buffers were purchased from Millipore Sigma. Amine functionalized agarose beads were purchased from Nanocs Inc. N-Succinimidyl 4-(maleimidomethyl) cyclohexane-1-carboxylate (SMCC) was purchased from Chem-Impex International, Inc. without further purification. NHS-Fluorescein (5/6-carboxyfluorescein succinimidyl ester), mixed isomer, was purchased from ThermoFisher Scientific.

### Preparation of LanM

The plasmid containing the gene encoding for LanM-GSGC was obtained from Twist Bioscience (pET-29b(+)-LanM-GSGC). The gene sequence consisted of the codon-optimized wt-LanM sequence with (ggcagcggctgc) inserted before the stop codon.²⁰ The protein was overexpressed in E. coli BL21(DE3) cells (NEB) at 37° C. after induction with 0.2 mM IPTG at an OD₆₀₀ₙₘ of 0.6. Purification was carried out as described except all buffers contained 5 mM TCEP.³⁹ Briefly, cells were lysed and loaded to a 25-mL (2.5×5.0 cm) Q-Sepharose Fast Flow column and eluted using a 0.01-1 M NaCl gradient. LanM-GSGC containing fractions were purified further using gel filtration chromatography (HiLoad 16/600 Superdex 75 pg column (120 mL) in 30 mM MOPS, 100 mM KCl, 5 mM CaCl₂, 5% glycerol, 5 mM TCEP, pH 7.0. LanM-GSGC-containing fractions were exchanged by FPLC into 20 mM acetate, 10 mM EDTA, 100 mM NaCl, 5% glycerol, pH 4.0, concentrated to ˜3 mM, and frozen in liquid N2 for storage.

### Maleimide Functionalization of Aqarose Beads

Amine functionalized agarose microbeads (1.2 ml) were transferred into a 5 mL Eppendorf tube and washed with pH 7.4 phosphate-buffered saline (PBS) three times and resuspended at a final volume of ˜1.7 mL (1.2 mL microbeads and 0.5 mL PBS supernatant). 0.15 g SMCC was dissolved in 3.4 mL DMSO and then combined with the microbeads. After 2.5 h incubation on a rocker mixer at room temperature, the functionalized agarose microbeads were washed with DMSO three times to remove unreacted SMCC and the washed with coupling buffer (50 mM HEPES (pH 7), 50 mM KCl, and 10 mM ethylenediaminetetraacetic acid (EDTA)) three times to remove DMSO solvent. The maleimide-microbeads were then used for LanM immobilization within 2 h.

### LanM Immobilization

LanM immobilization was carried out using a thiol-maleimide click chemistry conjugation reaction. Specifically, right before immobilization, the LanM-GSGC protein was buffer exchanged into the pH 7.0 coupling buffer (50 mM HEPES, 50 mM KCl, and 10 mM EDTA) using VivaSpin®2 centrifugal concentrators (molecular weight cut-off 3,000 g/mol, GE Healthcare), which yielded a final protein concentration of ˜2 mM. Then 2 mL of LanM solution was combined with 1 mL of maleimide-microbeads and the conjugation reaction was run for 16 h at room temperature. The unconjugated LanM proteins were removed by washing with pH 7 coupling buffer and the LanM-microbeads were stored in pH 7 coupling buffer for subsequent tests. The maleimide-microbeads were also incubated with coupling buffer without LanM protein as an unconjugated control sample.

### Breakthrough Column Experiments

Econo-Column glass chromatography columns (Bio-Rad; 5 cm×0.5 cm) were filled with MilliQ water (18.2 MΩ cm⁻¹) before LanM-microbeads were added gravimetrically. Columns were washed with 25 mM HCl, MilliQ water, and conditioned with buffer solution (same buffer for breakthrough experiment, see below) before conducting breakthrough experiments. REE stock solutions were prepared by dissolving individual REE chloride salts in 1 mM HCl. The stock solutions were diluted either in 10 mM buffer (pH 5: Homopiperazine-1,4-bis(2-ethanesulfonic acid, Homo-PIPES); pH 4: acetate acid; pH 3.5-2.2: glycine) or HCl at the desired pH. The REE solutions were pumped at 0.5 mL/min unless otherwise specified and the column effluent was collected in 1.0 mL aliquots. For single REE ion solution, REE ion concentrations were quantified by Arsenazo Ill assay. Specifically, 40 μL of sample was combined with 40 μL of 12.5 wt. % trichloroacetic acid (TCA) and then added to 120 μL of filtered 0.1 wt. % Arsenazo in 6.25 wt. % TCA. Absorbance at 652 nm was measured and compared to standards to determine the REE metal ion concentrations. The accuracy of the colorimetric assay was also confirmed in previous work by ICP-MS. For experiments with REE mixture or leachates, the metal ion concentrations were determined by ICP-MS.

For REE pair separation experiment, the metal ion purity is defined as

$${Purity}_{REE 1} = \frac{{C}_{REE 1}}{{C}_{REE 1} + {C}_{REE 2}}$$

Where C_{REE1} and C_{REE2} are molar concentration of REE1 and REE2, respectively. Separation factor

$${β}_{i j} = \frac{{Y}_{i} / {Y}_{j}}{{X}_{i} / {X}_{j}}$$

Where Yᵢ and Yⱼ are molar concentration of metal ions i and j in eluent, respectively. Xᵢ and Xⱼ are molar concentration of metal ions i and j in feed, respectively.

To determine the intra-REE selectivity of lanmodulin, the REE distribution factor (D) between the LanM phase (bound) and solution phase (unbound) is calculated as:

$$D = \frac{{[ M ]}_{LanM}}{{[ M ]}_{Liquid}}$$

where [M]_{LanM} and [M]_{Liquid} is the metal molar concentration in the LanM phase and solution phase at equilibrium, respectively. Note that [M]_{liquid}=[M]_{ad}; However, [M]_{LanM}=([M]_{de}*4-[M]_{ad}*0.8)/4, as free REE cannot be removed completely from the adsorption process.

The separation factor (SF) is defined as:

$$S F = \frac{{D}_{REE 1}}{{D}_{REE 2}}$$

where D_{REE1} and D_{REE2} are distribution factor of REE1 and REE2, respectively. To normalize D values collected from two batches, the following equation is used:

$$log {SF}_{\frac{1}{2}} = log \frac{{D}_{REE 1}}{{D}_{REE 2}} = log {D}_{REE 1} - log {D}_{REE 2}$$

LanM Labelling with Fluorescein

LanM (0.3 mL, 2 mM) was exchanged into 4-(2-hydroxyethyl)-1-piperazineethanesulfonic acid (HEPES) buffer (10 mM; pH 7) with 100 mM KCl and 8 mM Nd³⁺ using a spin column (Zeba™ Spin Desalting Columns, 7K MWCO, ThermoFisher Scientific). Fluorescein NHS ester (1 mg) was dissolved in dimethyl sulfoxide (DMSO, 30 μL) and added into the LanM solution at ˜3 mg/mL final concentration. After a 3 h incubation at room temperature, the untagged dyes were removed using a spin column (Zeba™ Spin Desalting Columns, 7K MWCO, ThermoFisher Scientific).

### Confocal Microscopy

For confocal microscopy, fluorescein labeled LanM (F-LanM) was used for immobilization on agarose microbeads. The microbeads were then dropped on a microscope glass coverslip along with a drop of VECTASHIELD® antifade mounting medium (Vectorlabs). The samples were imaged using a Zeiss LSM 710 confocal microscope equipped with a 40× NA 1.1 water immersion microscope objective. Airyscan mode was used to acquire 3D fluorescence images of LanM-agarose.

### Arsenazo III Assay for Sc and Nd Quantification

Sc and Nd concentrations in batch experiments and breakthrough curves were determined using the Arsenazo Ill assay. Specifically, for Sc, 80 μL of sample was combined with 100 μL of 0.2 M pH 2.8 glycine buffer and then added to 20 μL of filtered 0.3 wt. % Arsenazo in 0.2 M glycine. For Nd, 40 μL of sample was combined with 40 μL of 12.5 wt. % trichloroacetic acid (TCA) and then added to 120 μL of filtered 0.1 wt. % Arsenazo in 6.25 wt. % TCA. Absorbance (Nd at 652 nm; Sc at 675 nm) was measured and compared to standards to determine the metal ion concentrations. The accuracy of the colorimetric assay was also confirmed in previous work by ICP-MS.

### Spectrofluorometric Titrations of Wt-LanM

A solution of 20 μM Chelex-treated LanM was prepared in 20 mM acetate, 100 mM KCl, pH 5.0. In experiments that began with metalated LanM, 40 μM (2 equivalents) of metal was also added. The protein solution (600 μL) was placed in a 10-mm quartz spectrofluorometry cuvette (Starna Cells, 18F-Q-10-GL14-S) and assayed using a PerkinElmer fluorescence spectrometer FL 6500 in kinetic mode (80 kW power, 278 nm excitation, 2.5 nm excitation slit width, 307 nm emission, 5 nm emission slit width). Titrations were carried out through addition of at least 0.6 μL of titrant (either 10 mM metal or 1 mM citrate, pH 5.0) followed by ˜1 min of signal equilibration. For each data point, 10 s of signal were averaged after equilibration. These values were corrected for dilution and normalized to F₃₀₇ₙₘ=1 for the apoprotein.

### Inductively Coupled Plasma Mass Spectrometry (ICP-MS)

The elemental composition of the multi-element synthetic solutions and leachate solutions were determined using an ICP-MS. In the case of Dy/Nd binary synthetic solution, ICP-MS analyses were performed on an ICP-MS. All samples and calibration standards were acidified using concentrated nitric acid (70 wt %, purity≥99.999% trace metals basis, Sigma-Aldrich) to 2.5% (v/v), and spiked with internal standards of In, Rh, and Bi to adjust for shifts in signal intensity during analysis. Samples were analyzed in both hydrogen (for Ca and Si) and helium (for all other elements) reaction gas mode to reduce doubly charged and polyatomic interferences. Due to the complex matrix of coal ash leachate, it was necessary to use correction equations to adjust for mass interferences with ⁴⁵Sc and ¹⁵³Eu. A single replicate was used for the column breakthrough tests given the high sampling frequency throughout the breakthrough curves. All feed solutions were performed in triplicate. To describe the experimental uncertainty of metal ion quantification in complex synthetic solutions and leachate solutions, a coefficient of variation for each metal ion concentration was determined based on the triplicate of feed solutions.

### Leachinq and PH Adjustment of PRB Coal Ash

Leaching and pH adjustment of PRB coal ash was performed according previous works.^{34,35} Briefly, a PRB coal fly ash sample collected in 2017 from a pulverized coal-fired power plant was leached in 40 mL of a 1 M HCl solution for 4 h at 85° C. at a pulp density of 100 g/L. The leachate was cooled to room temperature and centrifuged at 3000 g for 15 min to remove any undissolved particles. The supernatant of leachate solution was then collected and received dropwise additions of a 10 M NaOH stock solution until a desired endpoint pH value was achieved (as monitored by a combination pH electrode). The pH adjusted leachate was centrifuged again to remove precipitates formed during pH adjustment and then filtered using a 0.2 μm polypropylene filter.

### Results and Discussion

Immobilization of LanM onto Aqarose Microbeads

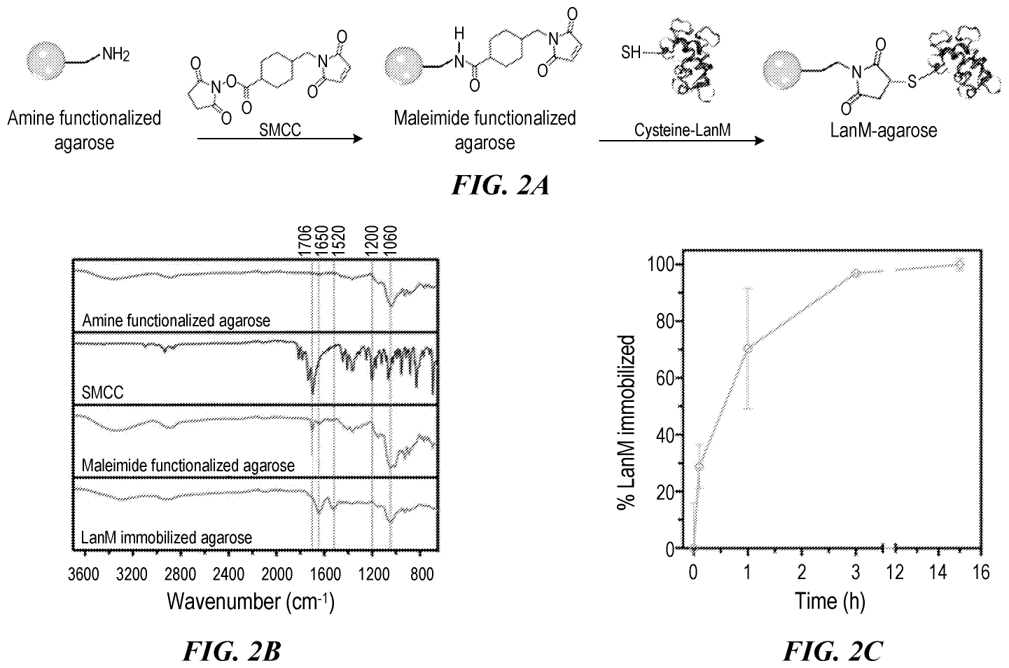

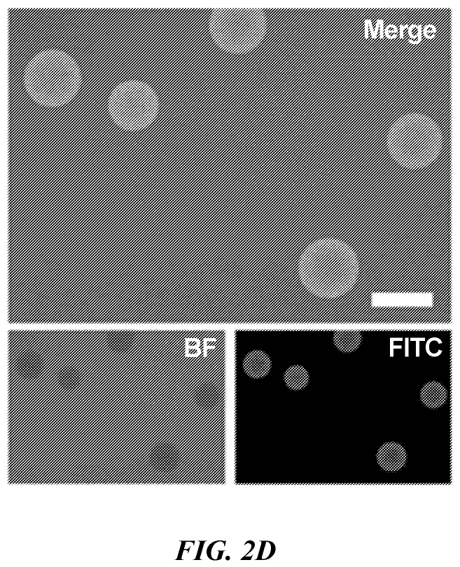

To facilitate the application of LanM for REE recovery in a flow-through format, a LanM variant containing a C-terminal cysteine residue with a GSG spacer (hereafter “LanM”) was immobilized on agarose microbeads using thiol-maleimide click chemistry (FIG. 2A).^{22,23} Compared with other immobilization strategies, such as physical binding and entrapment, site-specific covalent attachment enables stable and surface-accessible protein display, which is desirable for repeated cycles of adsorption/desorption under harsh conditions (e.g., low pH and high ionic strength). Thiol maleimide chemistry was pursued given the ease of encoding a terminal cysteine residue and its stability at low pH (vide infra). Purified LanM was handled at low pH (see methods) to preclude sulfur oxidation while avoiding the use of a reducing agent, which was found to adversely affect LanM immobilization (data not shown). Maleimide and LanM functionalization of the agarose beads was confirmed by FT-IR (FIG. 2B) and the immobilization kinetics were monitored by quantifying the free LanM concentration in the conjugation solution over a 16-h conjugation reaction. As shown in the FIG. 2B, the 1060 cm⁻¹ peak was assigned to a glycosidic bond that forms the backbone of the agarose polymer. In the SMCC spectrum, the peaks located at 1200 cm⁻¹ and 1706 cm⁻¹ were ascribed to succinimide and maleimide groups, respectively. After reaction of amine-functionalized agarose with SMCC, the presence of a 1706 cm⁻¹ peak and the lack of a 1200 cm⁻¹ peak indicates successful maleimide functionalization. Lastly, after the thiol-maleimide click chemistry, the LanM-agarose spectrum showed two peaks at 1650 cm⁻¹ and 1520 cm⁻¹, which are correspond to Amide I and Amide II, respectively, suggesting the immobilization of LanM proteins.

Approximately 97% of the added LanM was loaded within 3 h (FIG. 2C), resulting in an immobilization density of 2.89±0.34 μmol LanM/mL agarose. To visualize the distribution of LanM within the agarose bead, a fluorescently tagged variant of LanM (FITC-LanM, green) was incorporated during immobilization. Confocal microscopy imaging confirmed a homogenous distribution of FITC-LanM within the agarose microbeads (FIG. 2D).

### Immobilized LanM Retains the Ability to Bind REEs at Low PH and is Stable for Reuse

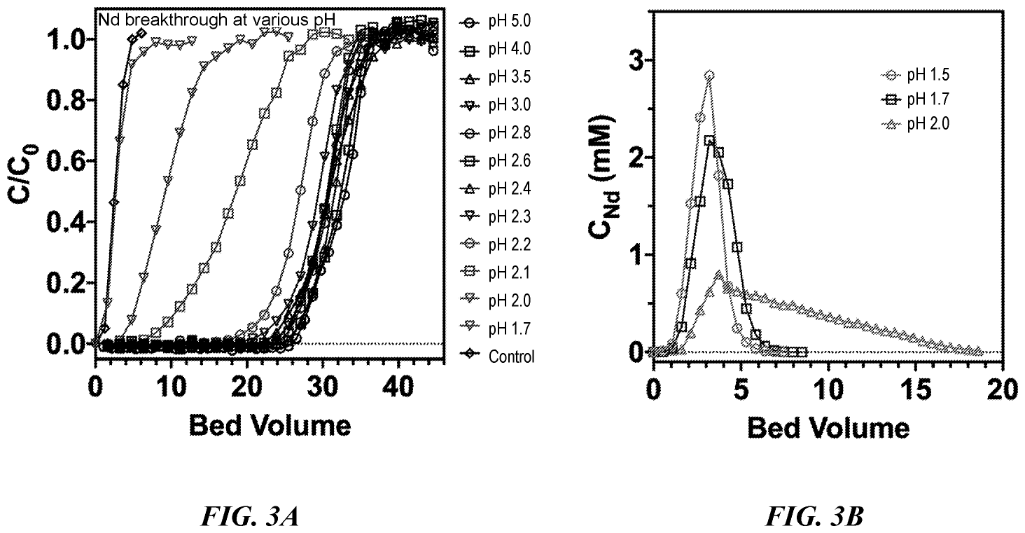

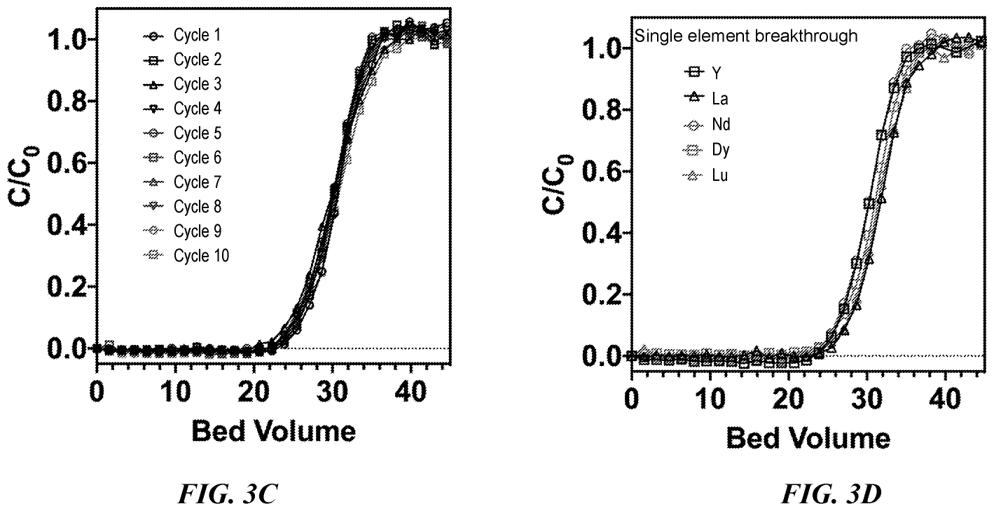

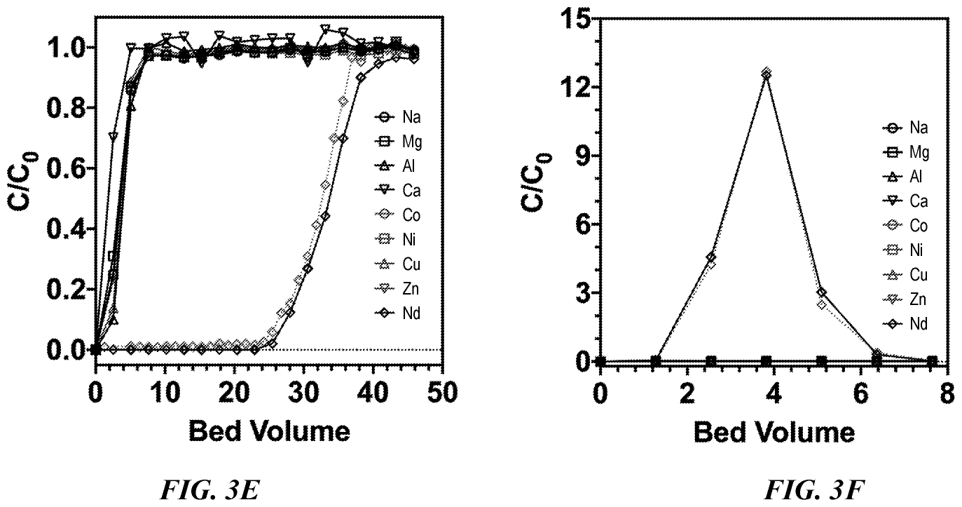

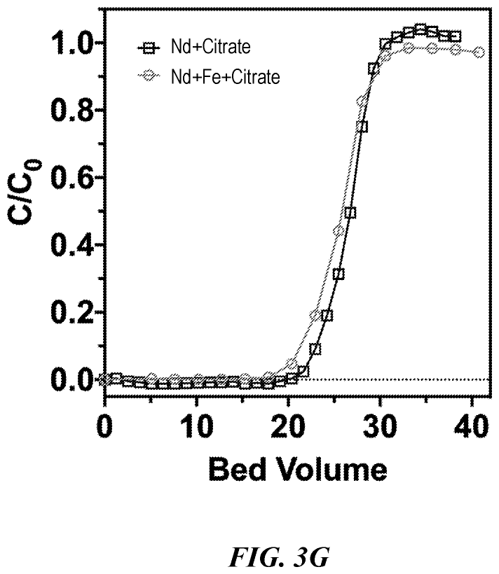

To test the efficacy of the immobilized LanM for REE extraction under flow through conditions, fixed-bed columns were packed with the LanM conjugates and influent breakthrough behavior was assessed with synthetic REE-containing solutions. Nd³⁺ was selected as a representative model REE system due to its abundance in REE deposits and high criticality for renewable energy technologies. The effect of pH on Nd breakthrough was assessed as shown in FIG. 3A (Experimental conditions: 0.2 mM Nd in 10 mM glycine buffer (pH>2.2 condition; For pH<2.1 conditions, Nd was diluted in HCl solution.), 0.5 mL/min flow rate; control experiment was performed at pH 3 by using a column packed with maleimide functionalized agarose). The Nd breakthrough point (at pH 5) occurred after ˜25 bed volumes, in contrast to 1 bed volume with non-conjugated agarose beads, which is due to the passage of the void volume, indicating that LanM retains high affinity for REE upon immobilization (FIG. 3A).

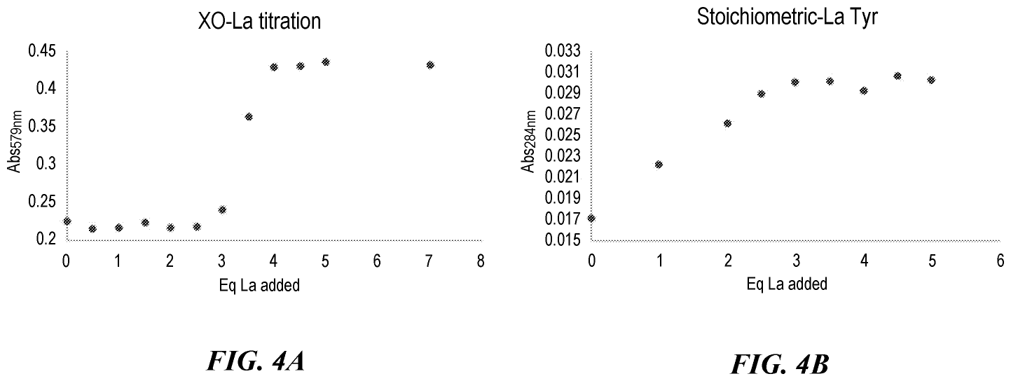

The adsorption capacity of the LanM column was 5.78 μmol/mL, which corresponds to a 2:1 stoichiometry of Nd per immobilized LanM. This contrasts with the absorption capacity of LanM in solution as shown in FIGS. 4A and 4B. In particular, stoichiometric titrations of LanM-Cys with La(Ill) demonstrate binding of 3 equivalents of REEs (FIGS. 4A and 4B) as evidenced by a competitive titration using xylenol orange as an indicator (FIG. 4A, titration performed in chelex-treated 20 mM MES, 100 mM KCl, pH 6.0 and LanM's conformational response to La(Ill) tracked as a shift in the A284 nm (FIG. 4B, titration performed in chelex-treated 30 mM MOPS, 100 mM KCl, pH 7). Interestingly, these results show that in solution, LanM binds 3 equivalent REEs (FIGS. 4A and 4B), suggesting that one metal site is destabilized upon immobilization, or in the column format.

Prior studies revealed that solubilized LanM can bind REEs at a pH as low as 2.5-3, based on this finding, the effect of influent pH on Nd extraction performance over a pH range of 1.7-5 was tested (FIG. 3A). The results indicate that immobilized LanM can effectively bind Nd down to pH 2.4, consistent with the results for solubilized LanM.¹⁶ Consistent with the behavior observed in solution, Nd binding to immobilized LanM is reduced by 50% at pH 2.2 and becomes insignificant at pH:51.7.

Taking advantage of the pH dependence of REE binding, Nd desorption was tested by pumping HCl solutions through Nd-saturated columns. The effect of pH (HCl concentration) on Nd breakthrough was assessed as shown in FIG. 3B (columns were pre-adsorbed with 40 bed volumes of 0.2 mM Nd at pH 3). A sharp desorption peak was observed between 1-6 bed volumes with pH at or below 1.7 with Nd concentrated by over an order of magnitude relative to the feed solution (FIG. 3B). In contrast, a pH 2.0 HCl solution yielded a tailed desorption profile with 16.5 bed volumes required to desorb >95% of the Nd. Importantly, the LanM-based sorbent was resilient to repeated low pH exposures given that 10 consecutive absorption/desorption cycles (pH 3.0 and 1.5, respectively) yielded no reduction in adsorption capacity (FIG. 3C, experimental conditions: flow rate of 0.5 mL/min. Desorption condition: 10 bed volumes of pH 1.5 HCl). To further determine the immobilized LanM's affinity to entire REE series, breakthrough curves with other representative REEs (i.e., Y, La, Dy, and Lu; FIG. 3D, experimental conditions: Feed: 0.2 mM) were performed at pH 3.0 and yielded indistinguishable results from that of Nd. Overall, the results demonstrate effective and reversible REE binding by immobilized LanM under low pH conditions.

### LanM Enables High-Purity Recovery and Concentration of REEs

To test the REE selectivity of immobilized LanM, Nd (0.2 mM) breakthrough experiments were conducted with a synthetic feed solution at pH 3.0 that contains millimolar levels of Mg²⁺, A1³⁺, Ca²⁺, C0²⁺, Ni²⁺, Cu²⁺, and Zn²⁺, which are abundant in REE-containing feedstocks.²⁴ The breakthrough of Nd occurred after 24 bed volumes (FIG. 3E, experimental conditions: metal ion breakthrough curves using synthetic feed solution containing 14 mM Na, 3.3 mM Mg, 9.1 mM Al, 2.3 mM Ca, 1.9 mM Co, 1.9 mM Ni, 1.8 mM Cu, 2.1 mM Zn, and 0.2 mM Nd at pH 3), whereas all non-REEs emerged with the void volume. Importantly, the Nd breakthrough curve and subsequent desorption curve were indistinguishable from the curves observed with a synthetic solution lacking non-REEs (FIG. 3F, gray diamonds indicate the Nd breakthrough/desorption profiles without competing non-REE ions, experimental conditions: desorption profile of metal ions was collected following treatment with pH 1.5 HCl), indicating that the behaviors of the REE and non-REE are completely decoupled in the process owing to the selectivity of LanM. Lastly, whereas the limited solubility of Fe³⁺ precluded its use in the multielement experiment, a breakthrough experiment using a binary Nd/Fe solution containing citrate to maintain Fe solubility through coordination, confirmed the selectivity of LanM for Nd³⁺ over Fe³⁺ (FIG. 3G, experimental conditions: Feed: Nd+Citrate: 0.2 mM Nd, 20 mM citrate, pH 3; Nd+Fe+Citrate: 0.2 mM Nd, 0.9 mM Fe, 20 mM citrate, pH 3). The ion concentration in the synthetic feeds for the data collected in FIG. 3G is enumerated below in Table 3.

TABLE 3
Ion Concentration of Synthetic Feed Solution (mM)

| Ion | Average | Standard deviation |
|---|---|---|
| Fe | 0.94 | 0.01 |
| Nd | 0.20 | 0.00 |

The selectivity of LanM for Nd over Fe³⁺ ions was studied separately because of (1) their low solubility at pH 3 condition (<100 μM) and (2) the instability of Fe³⁺ ions at between pH 3-4 range, which can form collide particles in the form of Fe(OH)₃. Previous studies showed that such collide particles, which may stably present as suspension in solution for up to 2 years, cannot be effectively removed even by 0.22 μm filters due to their small sizes. Additionally, such colloid particles may physically attach on to solid adsorbent materials and columns during flow-through experiments. To address this, 20 mM citrate was added to the Nd/Fe synthetic solution to improve the solubility of Fe³⁺ ions at pH 3 condition, allowing study of the REE selectivity of LanM over Fe³⁺. As shown in FIG. 3G, Nd breakthrough curves are almost identical with and without the presence of Fe³⁺, indicating LanM still have high REE selectivity over Fe³⁺ after immobilization. In practical, thanks to the lower Fe³⁺ solubility at higher pH (<10 μM at pH 4 and <0.01 μM at pH 5), Fe impurity can be largely removed by increasing pH to pH 4-5 range.

Collectively, these results suggest that immobilized LanM retains the high REE selectivity of LanM in solution and can be employed to separate REEs from non-REEs.

### Liqand Competition in Solution Reveals Potential for Intra-REE Separations

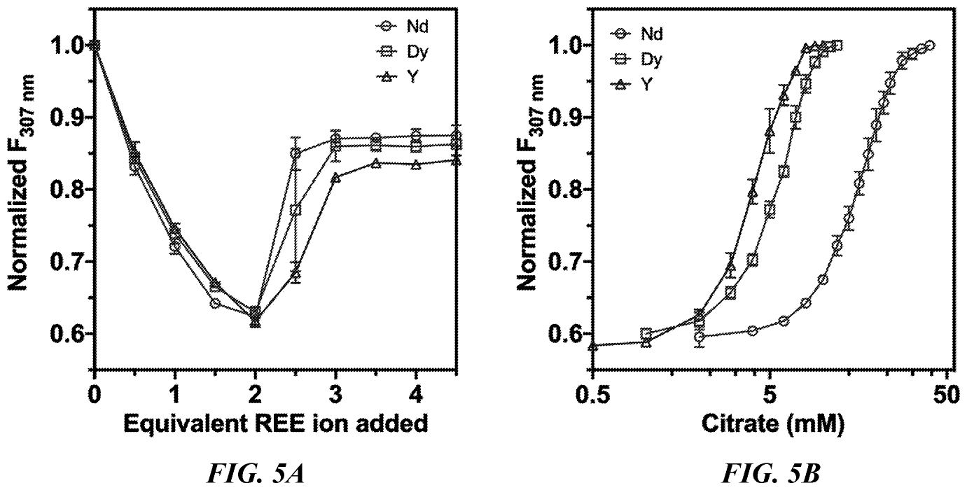

Initial characterization of LanM revealed that the protein exhibits an inverse affinity trend relative to most REE ligands, with highest, and roughly similar, affinity for La³⁺—Nd³⁺, and ˜5-fold lower affinity for Ho^{3+.20} However, previous studies of REE extraction in solution¹⁶ and on column (vide supra) have not yet attempted to exploit these differences for intra-REE separations. It was contemplated that competition with a mild chelator with a heavy REE preference, such as citrate,²⁵ could allow for enhanced selectivity in a desorptive process. To establish this principle, ligand competition with LanM in solution was first investigated. Taking advantage of the observation that the fluorescence of the sole tyrosine residue in LanM is quenched upon REE binding, which can be attributed to different solvent exposure and hydrogen bonding in the two protein conformations.²⁶ Titrations of LanM with REE displayed a maximal change in tyrosine fluorescence at 2 equiv. REE, which was counteracted upon binding of the third equivalent (FIG. 5A). It was posited that the REE-binding site responsible for this third equivalent also corresponds to the site destabilized upon immobilization. This fluorescence change provides a convenient handle to assay conditions for REE desorption from LanM. These studies, using individual titrations with Y, Dy, or Nd-bound LanM, revealed that citrate selectively outcompeted LanM for HREEs before LREEs, as expected given LanM's apparent K_{d}s (FIG. 5B). For example, 6 mM citrate is sufficient to almost fully desorb Dy from LanM, whereas Nd is still fully bound to the protein. The sharp desorption profiles (occurring over a 4-fold concentration range) are consistent with cooperativity in LanM's metal binding behavior.²⁰ These results demonstrate that, despite LanM's high REE affinity, mild chelators can be used to desorb REEs. Furthermore, and importantly, adjustment of conditions can be used to selectively desorb HREEs from LanM without destabilizing LREE-LanM complexes, setting the stage for on-column separations. The ion concentration in the synthetic feeds for the data collected in FIGS. 5A and 5B is enumerated below in Table 4.

TABLE 4
Ion Concentration in the Synthetic
Feed Solution (mM) in FIGS. 5A-5B

| | | | Coefficient |
|---|---|---|---|
| | | Standard | of variation |
| Element | Average | deviation | (%) |
| Na | 14.01 | 0.14 | 1.00 |
| Mg | 3.33 | 0.07 | 2.00 |
| Al | 9.18 | 0.09 | 1.00 |
| Ca | 2.32 | 0.19 | 8.12 |
| Co | 1.93 | 0.01 | 0.77 |
| Ni | 1.95 | 0.02 | 0.87 |
| Cu | 1.82 | 0.02 | 1.00 |
| Zn | 2.13 | 0.02 | 1.07 |
| Nd | 0.19 | 0.00 | 0.86 |

### Separation Between REE Pairs

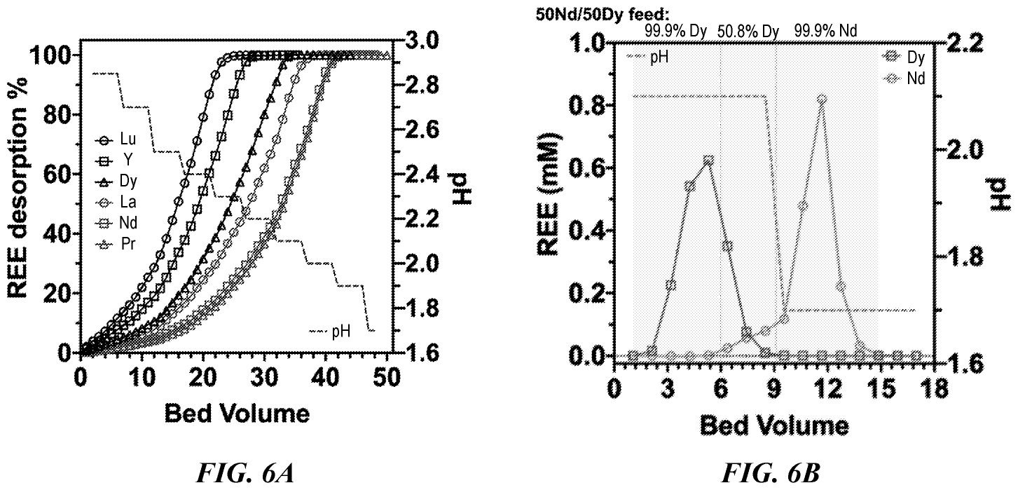

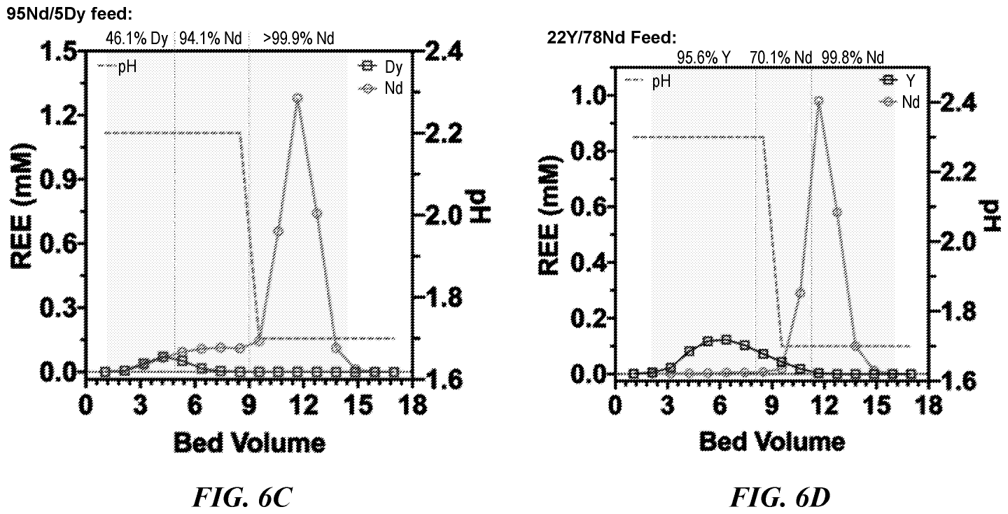

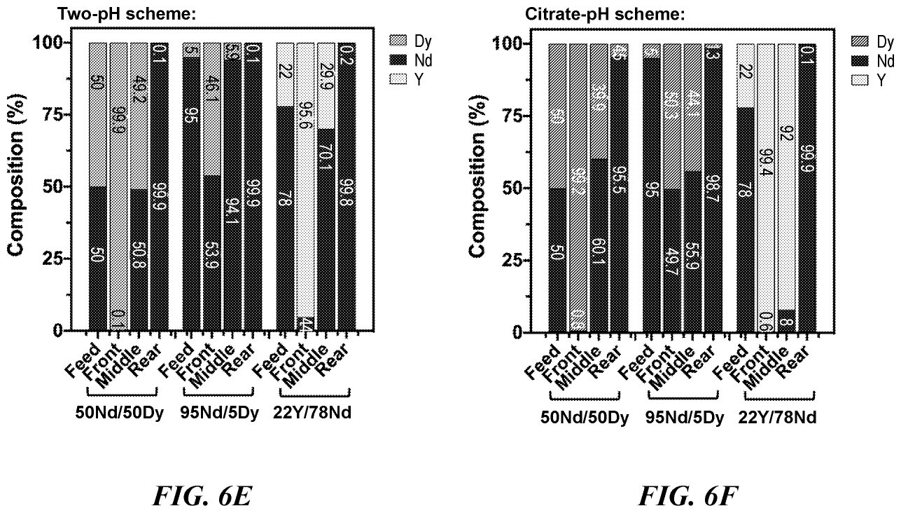

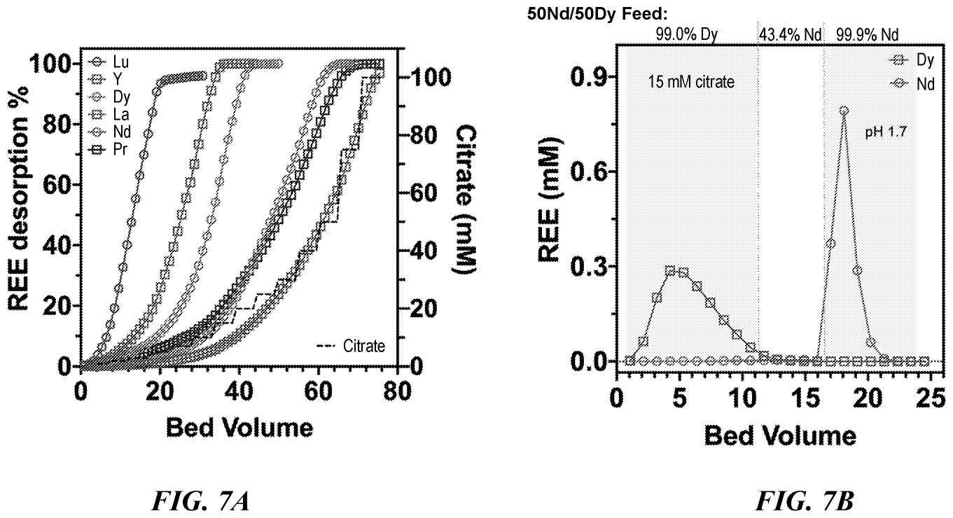

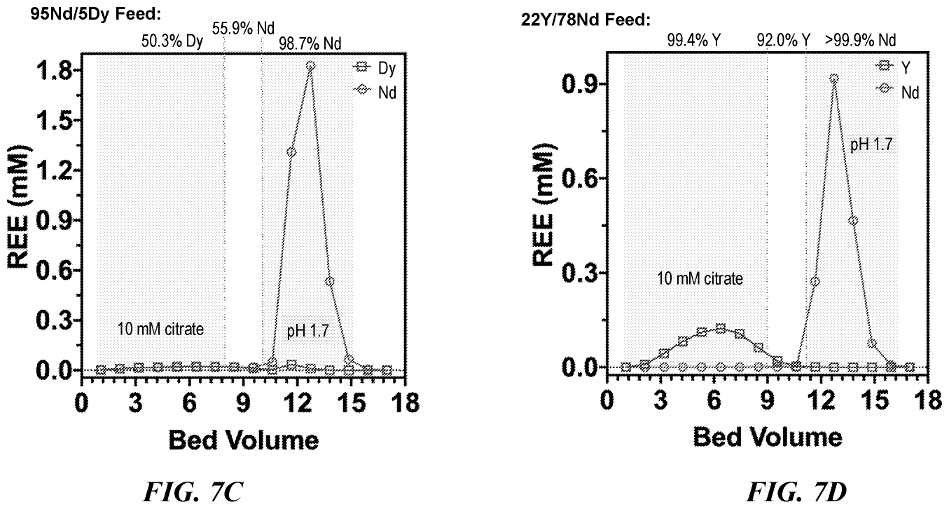

The next experiments examined whether judicious selection of desorption conditions could allow on-column separation among REEs, first using individual REE-loaded LanM columns over a range of pH (FIG. 6A, REE ion desorption was normalized to the total REE desorbed; experimental conditions: independent single REE solutions were used to load column to 90% saturation) and citrate steps (FIG. 7A, pH 5). A representative set of REEs (Y, La, Pr, Nd, Dy, and Lu) was tested independently to cover the entire ionic radius range of lanthanides (La and Lu) and based on their criticality for renewable energy technologies (Y, Pr, Nd, and Dy). Distinct elution profiles were observed for each REE under both desorption conditions (FIG. 6A and FIG. 7A). For citrate, the order of REE elution was correlated with the ionic radius and roughly consistent with the relative affinity of LanM-REE and citrate-REE complexes. A similar trend was observed for pH with the notable exception that Nd required a lower pH for desorption compared to La. This result is suggestive of higher relative stability for the LanM-Nd complex compared to LanM-La, and is consistent with a local maximum in stability for LanM in the Nd/Pr range.^{16,20} It was contemplated that the differences in stability among tested LanM REE complexes can be exploited to achieve separation between certain REE pairs.

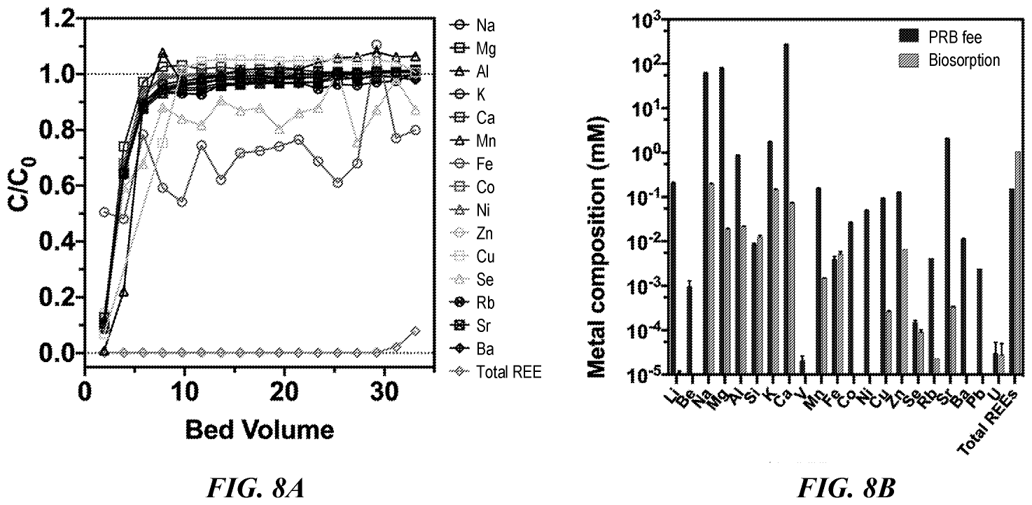

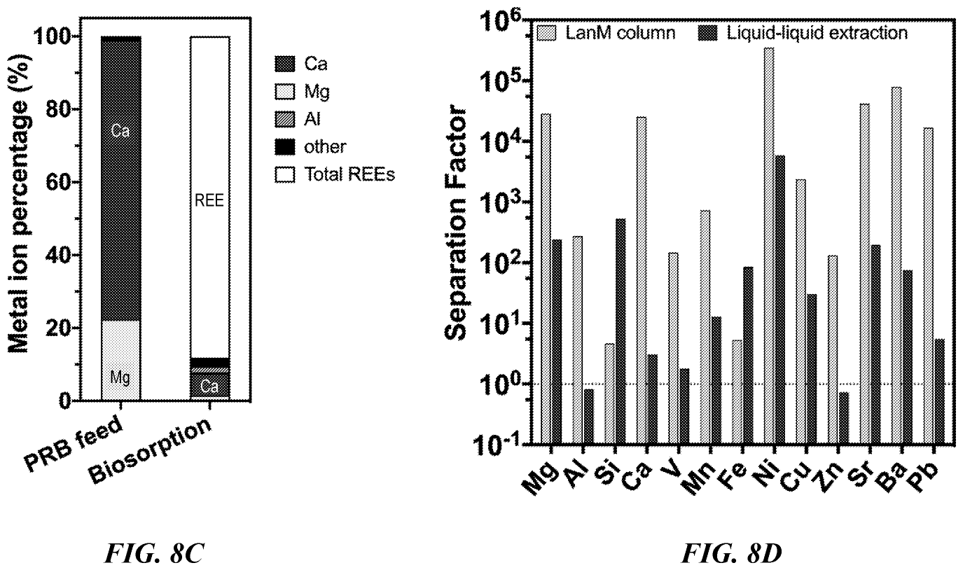

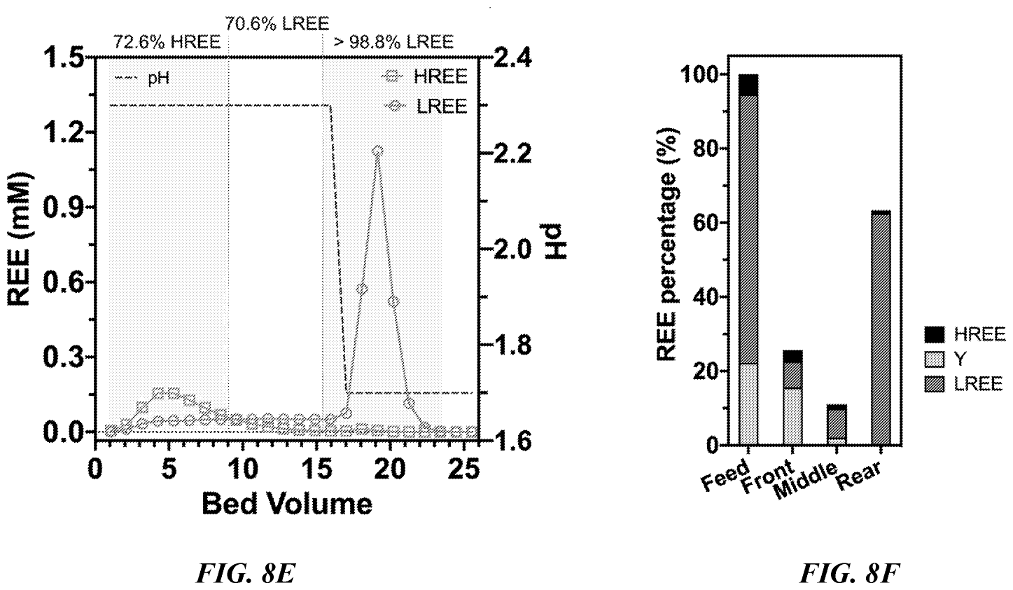

Single adsorption/desorption cycle for Dy/Nd separation. Guided by the above results, REE separation efficacy of LanM was tested by loading the column to 90% saturation with a solution of Nd and Dy at 50:50 molar ratio followed by a stage-wise desorption process (FIG. 6B, a feedstock comprised of a 50:50 mixture of Dy:Nd was loaded to 90% column saturation and then subjected to a two pH desorption scheme (2.1 and 1.7) and FIG. 7B, a feedstock comprised of a 50:50 mixture of Dy:Nd subjected to a two-step desorption scheme using 15 mM citrate (pH 5) followed by pH 1.7). Remarkably, Dy and Nd were eluted in separate fractions with high purity by employing either a two-pH desorption scheme or by combining an initial citrate-mediated Dy desorption following by pH-mediated elution of Nd. For the two-pH scheme, 76.2% of the Dy was eluted with 99.9% purity with an initial pH 2.1 desorption, while 76.8% of the Nd was eluted with 99.9% purity with a second pH 1.7 desorption. Less than <24% of the loaded REE material was present in the peak overlap region; considering the identical Nd/Dy composition as the feed solution (50.8% Dy; FIG. 8B), this fraction can be combined with the initial feed solution and processed during a future purification cycle (hence, avoiding any loss of REE). Desorption using 15 mM citrate eluted 94.2% of the adsorbed Dy at a purity of 99.1%, while a subsequent pH desorption step eluted the remaining Nd (99.2%) at a purity of 94.6%. Collectively, these results show that high purity Dy/Nd products can be obtained when starting with an equimolar mixture of both metal ions.

To test with a feedstock composition that reflects NdFeB magnet-bearing E-waste, the column was loaded to 90% of saturation with a feed solution comprised of 95% Nd and 5% Dy.²⁷ The use of Nd as a surrogate for the combined Nd/Pr content (typically 3:1 Nd:Pr) is supported by the nearly identical desorption profiles of both metal ions as a function of pH or citrate concentration (FIGS. 6A and 7A). Successful separation of Dy from Nd/Pr would enable the production of high-value dysprosium and didymium oxide products that could be fed back into the magnet manufacturing supply chain. As shown in FIG. 6C (a feedstock comprised of a 5:95 mixture of Dy:Nd loaded to 90% column saturation and then subjected to a two-pH desorption scheme (2.2 and 1.7)) and FIG. 7C (a feedstock comprised of a 50:50 mixture of Dy:Nd was loaded to 90% column saturation and then subjected to a two-step desorption scheme using 10 mM citrate (pH 5) followed by pH 1.7), both the two-pH and citrate desorption schemes yielded high-purity Nd fractions (99.8% and 98.7%, respectively) while significantly upgrading the Dy purity. For example, a pH 2.2 elution step yielded 88.6% of the Dy content at 46.1% purity while the citrate desorption step yielded 73.9% of the Dy at a purity of 48.9%.

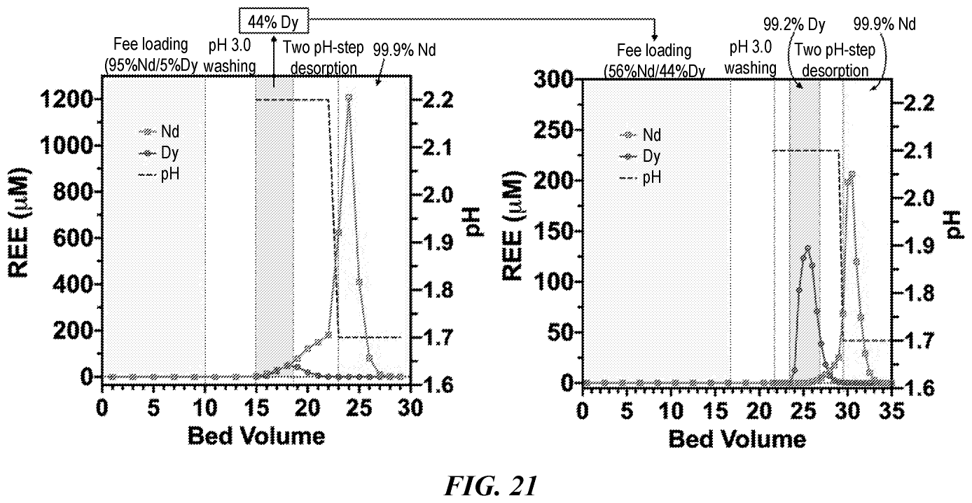

Two adsorption/desorption cycles for Dy/Nd separation. Given the successful Dy/Nd separation results with 50:50 mixtures (FIG. 6B), the roughly 50%-purity Dy fractions generated in an initial column step (i.e., a first adsorption/desorption cycle) from a 95% Nd/5% Dy feedstock (FIG. 6C) were tested to determine whether those fractions can be further upgraded to high purity by performing a second adsorption/desorption cycle. To this end, the Nd/Dy separation using the 95% Nd/5% Dy feed was repeated 5 times to accumulate sufficient volume of a 44% purity Dy desorption fraction to enable a second separation step (or cycle) (FIG. 21). Despite the dilute nature of the Nd/Dy feed solution, the LanM column exhibited a high loading yield (>99%) and achieved 88% of Dy at a purity of 99.2% and 82% of Nd at a purity of 99.9%, respectively, following the pH desorption step. These results suggest that the LanM column is able to effectively separate Dy from Nd starting with low-purity Dy solutions typical of E-waste leachates. An even higher yield and product purity is likely to be achieved with higher operational volumes. In addition, further REE separations can likely be realized upon fine tuning of column operation conditions, such as pH, competing chelator identity and concentration, flow rate, and column geometry.

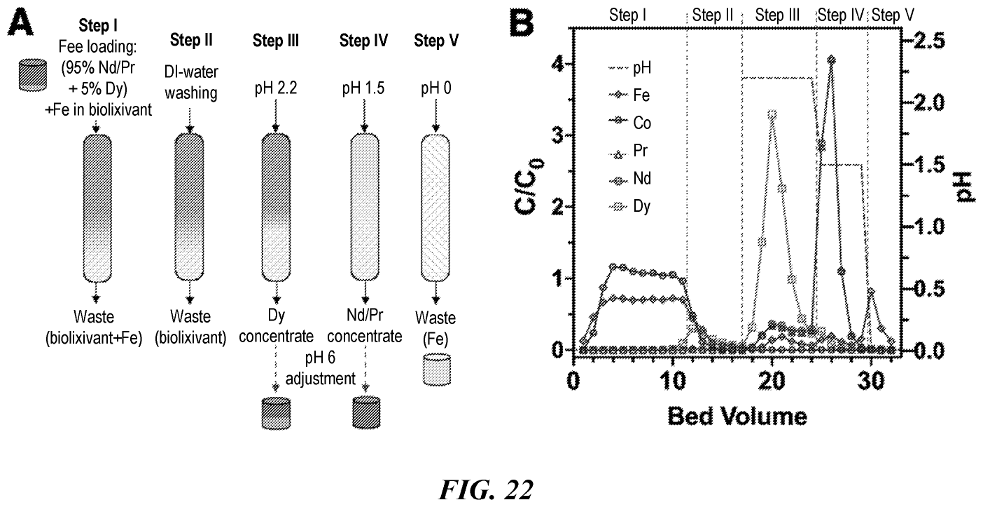

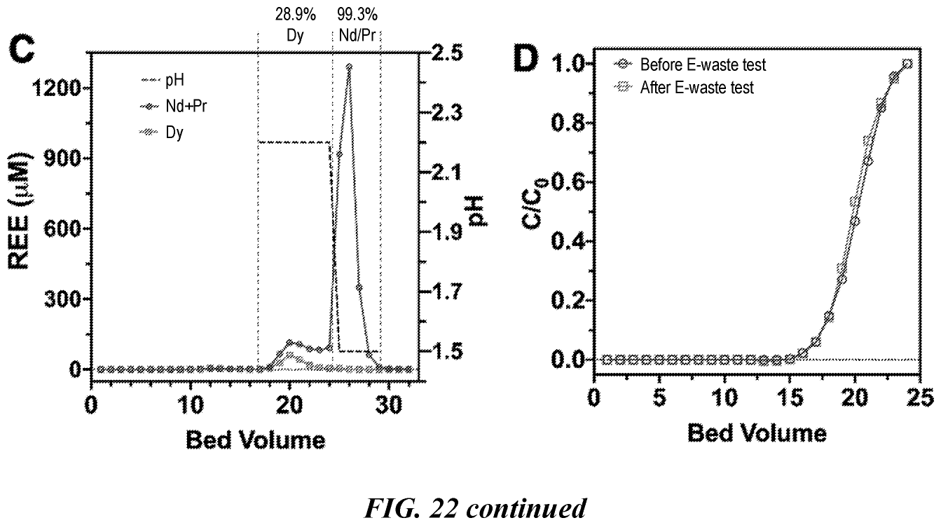

Dy/Nd separation with E-waste samples. Results with synthetic solutions suggested that LanM can effectively separate a 95% Nd/5% Dy feed into high purity Nd and Dy products in two adsorption/desorption cycles. To demonstrate the recovery and separation of REE from real E-waste, a stepwise separation scheme was tested using bioleachate prepared from HDD scraps (INL). After loading the E-waste bioleachate onto the LanM column (adsorption step), a distilled water washing step was used to remove the unbound metal ions and the biolixiviant. A 2-step desorption at pH 2.2 and pH 1.5 was then used to selectively desorb Dy and generate high-purity Nd/Pr, respectively. Finally, a 1 M HCl treatment step was used to remove Fe precipitates and regenerate the LanM column (FIG. 22A-B).

Using this scheme, Dy concentrate (28.9%; relative to total REE) and high purity Nd/Pr (99.3%) solutions were achieved. Based on prior results with synthetic solutions, it is likely that a second column step can be used to further upgrade the Dy fraction to high purity. A moderate level of Fe impurity was co-extracted in each desorption fraction, but previous work has indicated that this Fe can be effectively removed by precipitation, induced by adjustment of the solution to pH 6. The Fe remaining on the column could be removed by a 1 M HCl (pH 0) cleaning step (FIG. 22C). Lastly, the column stability was tested using a breakthrough curve with a 0.4 mM Nd solution (pH 3) with the regenerated column (FIG. 22D). No loss in REE adsorption capacity was observed. This study indicated that the LanM column can be used to process E-waste bioleachate to produce Dy concentrate and high purity Nd/Pr.

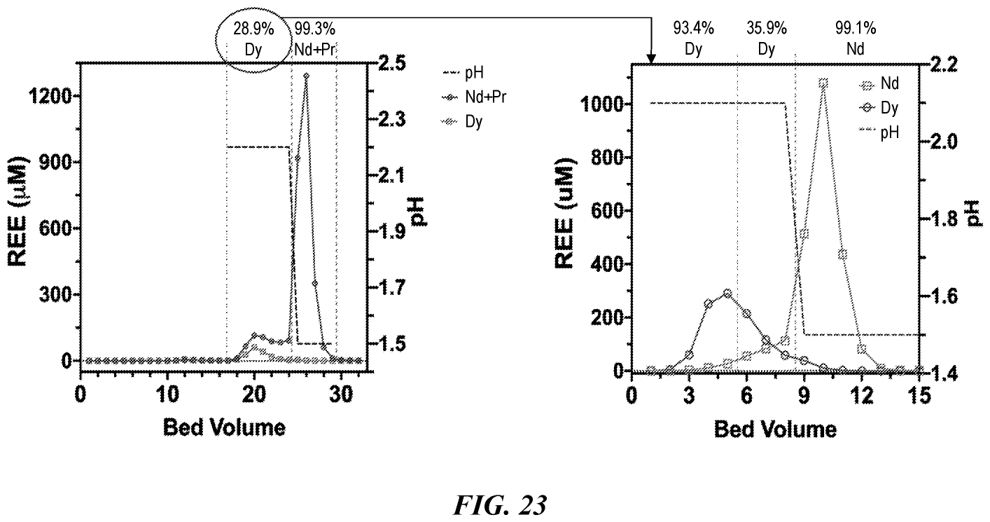

To test the efficacy of a second column step (i.e., a second adsorption/desorption cycle) to further refine the 28.9% Dy fraction into high-purity Dy and Nd, a synthetic 71% Nd/29% Dy feedstock solution was loaded onto the lanmodulin column and subjected to a two-step pH desorption process. High-purity Dy and Nd solutions were achieved (FIG. 23).

As compared to the immobilized LanM separation and purification processes discussed above, other extraction methods are not as effective. For example, in a recent liquid-liquid extraction study using bis(2,4,4-trimethylpentyl) phosphinic acid (Cyanex272) for Nd/Dy separation, a feed solution containing 6.7% Dy was concentrated to ˜47% purity Dy using a single extraction stage with 85.8% Dy extraction yield.²⁸ A separate study that employed a counter current extraction configuration with 2-ethylhexyl phosphonic acid mono-2-ethylhexyl ester (PC 88A) ligand required three and four extraction stages, respectively, to upgrade a 20% Dy /80% Nd feed solution to 76.7% and 95.2% purity Dy solutions.²⁹ As such, the immobilized LanM can be used as an effective and environmentally friendly alternative to liquid-liquid extraction to produce high purity (up to 99.9%) Nd and Dy separation.

Next, the ability of LanM to separate Y from Nd was tested (FIG. 6D, a feedstock comprised of a 22:78 mixture of Y:Nd was loaded to 90% column saturation and then subjected to a two-pH desorption scheme (2.3 and 1.7) and FIG. 7D, a feedstock comprised of a 22:78 mixture of Y:Nd was loaded to 90% column saturation and then subjected to a two-step desorption scheme using 10 mM citrate (pH 5) followed by pH 1.7), as both REEs are abundant in primary REE deposits (e.g., those bearing monazite, xenotime, and/or allanite), and are considered critical for green energy technologies. The proportion of Y and Nd in the feed solution was set to 22% and 78%, respectively, to resemble the HREE to LREE ratio in a typical coal byproduct leachate.^{30, 31} Two distinct peaks were collected after a two-step pH desorption, with 95.6% Y purity and 99.8% Nd purity achieved, respectively. A small overlap region (<25% of the adsorbed metals) of nearly identical composition as the influent feed solution (79.1% Nd+20.9% Y) was collected and can be recycled as feed solution in a subsequent adsorption/desorption cycle. Remarkably, the citrate/pH combination yielded nearly complete Y/Nd separation; 95.8% of Y was eluted at 99.4% purity while 99.7% of Nd was eluted with >99.9% purity. Thus, with a single adsorption/desorption cycle, Y can be separated from Nd. A summary of REE composition in feedstock and three desorption zones by using two-pH scheme and citrate-pH scheme is provided at FIGS. 6E and 6F, respectively. The ion concentration in the synthetic feeds for the data collected in FIGS. 6A-6F is enumerated below in Table 5.

TABLE 5
Ion Concentration in the Synthetic
Feed Solution (μM) in FIGS. 6A-6F

| | | | |
|---|---|---|---|
| | | | Coefficient |
| | | Standard | of variation |
| Ion | Average | deviation | (%) |

50/50 Feed

| | | | |
|---|---|---|---|
| Nd | 178.92 | 0.59 | 0.33 |
| Dy | 179.37 | 0.92 | 0.51 |

95/5 Feed

| | | | |
|---|---|---|---|
| Nd | 337.52 | 0.92 | 0.27 |
| Dy | 17.63 | 0.07 | 0.40 |

Grouped REE Extraction and Separation from a Low-Grade Feedstock Leachate

Recovering REEs from abundant waste products, such as coal fly ash and red mud, provides a potential means to diversify the REE supply while avoiding pollution inherent to mining. However, the leachate solutions produced from such low-grade REE bearing wastes, which contain high levels of metal ion impurities, such as A1³⁺, Ca²⁺, and Fe³⁺, are problematic for traditional liquid-liquid extraction approaches.^{5,24} For example, Al³⁺ is commonly co-extracted with REEs in liquid-liquid extraction, resulting in low REE purity and formation of emulsion via gelatinous hydroxides.²⁴ Similarly, other impurities, such as Ca²⁺, may cause fouling in liquid-liquid extraction processes through gypsum formation.³² As such, leachate solutions are commonly subjected to a pre-treatment precipitation step to remove impurities before feeding into a liquid-liquid extraction unit. Although selective precipitation is effective for removing certain impurities, such as Fe³⁺, the complete removal of A13+ presents a major challenge as REE hydroxides co-precipitate with aluminum hydroxide.^{24, 33} Therefore, a REE extraction method with high REE selectivity over non-REE impurities, particularly A13'⁰ and Ca²⁺, is highly desirable.

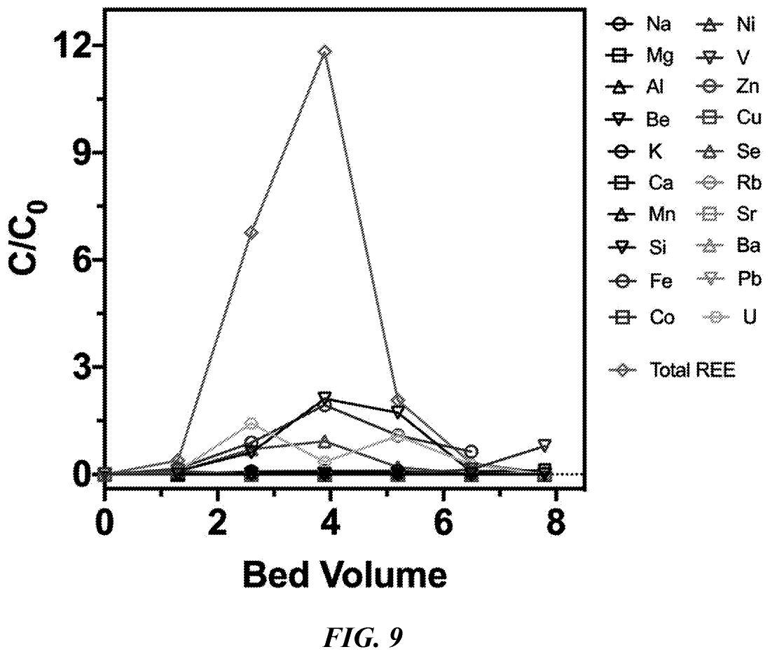

As an industrially relevant performance test, LanM column-based REE extraction and separation concepts were demonstrated using a low-grade leachate (0.043% REE, excluding monovalent ions) prepared from Powder River Basin (PRB) fly ash.^{34, 35} The leachate contains ˜150 μM total REEs compared with mM levels of Na, Mg, Al, Ca, and Sr (Tables 6A-6B), and significant transition metal content (e.g., Zn, Ni, Cu, and Mn). Using the LanM-based column, the breakthrough of REEs occurred after 30 bed volumes, whereas non-REEs were eluted in the void volume (FIG. 8A, adsorption profiles of metal ions; adsorption condition: pH 5, 0.5 mL/min. 1 bed volume=0.8 mL). To assess the REE purity of the adsorbed metal content, the metal ion composition was determined following non-selective desorption using a pH 1.5 solution. Over 96.5% of the REEs were desorbed within the most concentrated fractions (total 3.9 bed volumes), with an average enrichment factor of 6.9 (FIG. 8B and FIG. 9, metal compositions in PRB feed and recovered biosorption solutions). A total REE purity of 88.2% was observed (FIG. 8C, metal ion percentage in PRB feed and recovered biosorption solutions (excluding monovalent ions)), indicating a striking 2050-fold increase in purity compared with the feed solution (0.043% REEs, excluding monovalent ions). Importantly, the radionuclide uranium was not concentrated from the PRB leachate.

TABLE 6A
Ion concentration in PRB feed solution (μM) at pH 5

| | | | Coefficient |
|---|---|---|---|
| | | Standard | of variation |
| Ion | Average | deviation | (%) |
| Li | 203.010 | 9.856 | 4.86 |
| Be | 0.963 | 0.325 | 33.71 |
| Na | 61626.204 | 989.812 | 1.61 |
| Mg | 80210.044 | 1178.194 | 1.47 |
| Al | 858.709 | 16.698 | 1.94 |
| Si | 8.503 | 0.594 | 6.98 |
| K | 1739.691 | 32.054 | 1.84 |
| Ca | 273173.440 | 1477.705 | 0.54 |
| Sc | 0.069 | 0.009 | 12.59 |
| V | 0.021 | 0.005 | 23.88 |
| Mn | 157.649 | 1.738 | 1.10 |
| Fe | 3.990 | 0.546 | 13.68 |
| Co | 27.054 | 0.165 | 0.61 |
| Ni | 50.702 | 0.421 | 0.83 |
| Cu | 94.046 | 0.577 | 0.61 |
| Zn | 127.133 | 0.324 | 0.26 |
| Se | 0.151 | 0.015 | 10.07 |
| Rb | 4.176 | 0.002 | 0.04 |
| Sr | 2057.752 | 9.659 | 0.47 |
| Y | 35.326 | 0.136 | 0.38 |
| Ba | 11.503 | 0.067 | 0.58 |
| La | 23.636 | 0.060 | 0.25 |
| Ce | 46.504 | 0.156 | 0.34 |
| Pr | 6.300 | 0.027 | 0.42 |
| Nd | 24.453 | 0.114 | 0.47 |
| Sm | 4.467 | 0.040 | 0.89 |
| Eu | 0.987 | 0.003 | 0.27 |
| Gd | 4.188 | 0.016 | 0.38 |
| Tb | 0.600 | 0.004 | 0.69 |
| Dy | 3.448 | 0.020 | 0.58 |
| Ho | 0.648 | 0.005 | 0.75 |
| Er | 1.780 | 0.016 | 0.92 |
| Tm | 0.224 | 0.001 | 0.28 |
| Yb | 1.256 | 0.020 | 1.59 |
| Lu | 0.185 | 0.004 | 1.93 |
| Pb | 2.443 | 0.004 | 0.15 |
| Th | 0.000 | 0.000 | N/A |
| U | 0.030 | 0.023 | 38.84 |

TABLE 6B
Ion concentration (μM) in the three most
concentrate fractions of PRB desorption solution

| Ion | Average* |
|---|---|
| Li | 0.011 |
| Be | 0.000 |
| Na | 199.627 |
| Mg | 19.309 |
| Al | 21.718 |
| Si | 12.628 |
| K | 148.153 |
| Ca | 74.565 |
| Sc | 0.339 |
| V | 0.000 |
| Mn | 1.487 |
| Fe | 5.206 |
| Co | 0.000 |
| Ni | 0.000 |
| Cu | 0.272 |
| Zn | 6.660 |
| Se | 0.092 |
| Rb | 0.023 |
| Sr | 0.339 |
| Y | 10.956 |
| Ba | 0.000 |
| La | 177.838 |
| Ce | 448.001 |
| Pr | 63.554 |
| Nd | 247.153 |
| Sm | 44.894 |
| Eu | 9.838 |
| Gd | 36.405 |
| Tb | 4.307 |
| Dy | 14.679 |
| Ho | 1.200 |
| Er | 1.506 |
| Tm | 0.067 |
| Yb | 0.346 |
| Lu | 0.050 |
| Pb | 0.000 |
| Th | 0.000 |
| U | 0.028 |

*The uncertainty for each element was assumed based on the triplicate test from Table 5A.

To benchmark the REE selectivity of the LanM column-based method with a traditional liquid-liquid extraction approach (FIG. 8D, values above dot line suggest higher REE affinity over non-REE), the separation factor for total REEs relative to base metal ions was compared with data generated using the widely commercial extractant, Di-2-ethyl-hexylphosphoric acid (DEHPA) with coal fly ash leachate of a comparable composition.³⁶ While the LanM-based approach exhibited higher selectivity against all non-REE impurities with the exception of Fe and Si, the orders of magnitude higher selectivity for Mg²⁺, A13+, and Ca²⁺ is particularly compelling considering their abundance low-grade feedstock leachates.^{31, 37} It is contemplated that the lower relative selectivity against Si and Fe reflects the formation of unfilterable colloid particles that accumulate on column and are dissolved during the low pH desorption step. As such, the current column-based approach necessitates pre-column methods to remove Fe/Si content in order to maximize the effect of LanM. In sum, these results highlight the ability of the immobilized LanM to selectively concentrate REEs from low-grade leachates containing a wide array of metal ion impurities.

Following the demonstration of effective removal of the vast majority of non-REE impurities during the adsorption stage, the ability of LanM to enable grouped separation of the REEs adsorbed from the PRB leachate (LREE (La−Gd): 72%; HREE (Tb−Lu+Y): 28%) using a two-pH desorption scheme was tested. Grouped separation of REEs into HREE and LREE fractions is an important early step during liquid-liquid extraction and requires multiple extraction and stripping stages to enrich for HREEs and typically involves the use of a different extractant compared to that used in the non-REE impurity removal stage.⁵ Two distinct peaks comprised predominately of either HREEs or LREEs were observed (FIG. 8E); 82% of the HREEs were eluted with 72.6% purity at pH 2.3, while 80% of the LREEs were eluted with 98.8% purity at pH 1.7 desorption. Sc was also co-extracted with other REEs and desorbed with LREE group; however, its concentration was particularly low in the PRB leachate (0.02 μM). To achieve a similar purity LREE separation from an acid leachate generated from ion adsorption, 11-stages of counter-current extraction were required through a stepwise liquid-liquid extraction process, where two common extractants, 2-ethyl-hexyl phosphonic acid mono-2-ethylhexyl ester (HEH(EHP), P507) and di-(2-ethyl hexyl)phosphoric acid (HDEHP, P204), were exploited sequentially.³⁸ Collectively, these results highlight a key advantage of the LanM column: the ability to achieve both non-REE impurity removal and grouped REE separation with low-grade leachates in a single adsorption/desorption step without using organic solvent or hazardous chemicals. Based on the separation data with Nd/Dy, and Nd/Y pairs, finer separation within the HREE and LREE groups is likely possible by linking multiple adsorption/desorption steps and/or by judicious incorporation of organic chelators in the desorption step. Selective desorption of HREE (Tb−Lu+Y) and LREE (La−Gd) by a two-step pH scheme are shown in FIG. 8E (the values above panel D indicate the purity of LREE or HREE regarding total REE contents, three elution zones are divided by vertical dot lines) and the REE ratio regarding total REE in PRB feed and three elution zones is shown in FIG. 8F (experimental conditions: 29.1 bed volumes of PRB fly ash leachate was pumped through a 0.94 mL column, followed by a washing step with 10 bed volumes of pH 3.5 water. Desorption was performed with 16 bed volumes of pH 2.3 HCl solution and 10 bed volumes of pH 1.7 HCl solution, sequentially). The ion concentration in the synthetic feeds for the data collected in FIGS. 8A-8D is enumerated below in Table 7 and the ion concentration (pM) in the PRB leachate feed, and three desorption zones in FIGS. 8E and 8F is enumerated below in Table 8.

TABLE 7
Ion concentration of synthetic feed solution (μM) in FIGS. 8A-8D

| | | | Coefficient |
|---|---|---|---|
| | | Standard | of variation |
| Ion | Average | deviation | (%) |
| Y | 74.81 | 0.97 | 1.30 |
| Nd | 255.74 | 2.36 | 0.92 |

TABLE 8
Ion concentration (μM) in the PRB leachate
feed, and three desorption zones in FIG. 8E and 8F

| | Feed | | | | | |
|---|---|---|---|---|---|---|
| | | | Coefficient | | | |
| | | Standard | of | Front | Middle | Rear |
| Ion | Average | deviation | variation (%) | — | — | — |
| Li | 221.220 | 8.278 | 3.742 | — | — | — |
| Be | 1.247 | 0.057 | 4.530 | — | — | — |
| Na | 83262.588 | 2974.199 | 3.572 | — | — | — |
| Mg | 84478.993 | 2756.163 | 3.263 | 5.246 | 0.743 | 0.044 |
| Al | 981.596 | 22.688 | 2.311 | 10.474 | 2.076 | 1.725 |
| K | 1910.022 | 55.005 | 2.880 | — | — | — |
| Ca | 126773.564 | 3260.582 | 2.572 | 7.645 | 1.500 | 1.030 |
| Sc | 0.021 | 0.000 | 1.089 | 0.012 | 0.009 | 0.045 |
| Ti | 546.018 | 13.212 | 2.420 | 0.318 | 0.290 | 0.291 |
| V | 0.009 | 0.000 | 1.530 | — | — | — |
| Cr | 0.028 | 0.001 | 3.869 | 0.015 | 0.015 | 0.100 |
| Mn | 165.665 | 3.303 | 1.994 | 0.044 | 0.024 | 0.019 |
| Fe | 3.011 | 0.128 | 4.244 | 1.103 | 0.644 | 0.889 |
| Cc | 26.208 | 0.119 | 0.455 | 0.003 | 0.000 | 0.001 |
| Ni | 48.771 | 0.689 | 1.413 | 0.375 | 0.012 | 0.043 |
| Cu | 82.732 | 1.478 | 1.786 | 1.977 | 0.122 | 0.108 |
| Zn | 107.892 | 1.259 | 1.167 | 0.226 | 0.214 | 0.236 |
| Se | 2.799 | 0.050 | 1.784 | 1.977 | 3.010 | 7.355 |
| Rb | 4.641 | 0.009 | 0.193 | 0.120 | 0.033 | 0.002 |
| Sr | 1929.365 | 4.871 | 0.252 | 0.684 | 0.076 | 0.032 |
| Y | 34.502 | 0.176 | 0.510 | 77.459 | 13.651 | 0.152 |
| Cd | 0.755 | 0.001 | 0.157 | 0.000 | — | 0.001 |
| Cs | 0.218 | 0.002 | 1.021 | 0.000 | — | 0.000 |
| Ba | 11.581 | 0.101 | 0.868 | 0.007 | 0.011 | 0.005 |
| La | 25.551 | 0.087 | 0.341 | 16.677 | 23.929 | 41.284 |
| Ce | 49.069 | 0.229 | 0.468 | 11.680 | 17.585 | 115.726 |
| Pr | 6.230 | 0.023 | 0.375 | 0.860 | 1.316 | 15.781 |
| Nd | 21.673 | 0.110 | 0.508 | 2.696 | 4.208 | 55.799 |
| Sm | 4.328 | 0.036 | 0.828 | 0.334 | 0.556 | 10.439 |
| Eu | 1.145 | 0.005 | 0.447 | 0.173 | 0.283 | 2.445 |
| Gd | 4.266 | 0.016 | 0.370 | 2.425 | 3.680 | 6.761 |
| Tb | 0.645 | 0.003 | 0.409 | 0.606 | 0.794 | 0.701 |
| Dy | 3.490 | 0.020 | 0.559 | 4.891 | 4.736 | 1.738 |
| Ho | 0.690 | 0.004 | 0.560 | 1.311 | 0.773 | 0.084 |
| Er | 1.835 | 0.008 | 0.460 | 3.951 | 1.257 | 0.071 |
| Tm | 0.235 | 0.002 | 0.665 | 0.579 | 0.078 | 0.001 |
| Yb | 1.243 | 0.005 | 0.428 | 2.943 | 0.211 | 0.016 |
| Lu | 0.189 | 0.003 | 1.410 | 0.457 | 0.013 | 0.004 |
| Pb | 2.165 | 0.024 | 1.113 | 0.749 | 0.004 | 0.002 |
| U | 0.024 | 0.000 | 1.049 | 0.057 | 0.002 | 0.002 |

Separation of REEs Using pH-Based Desorption

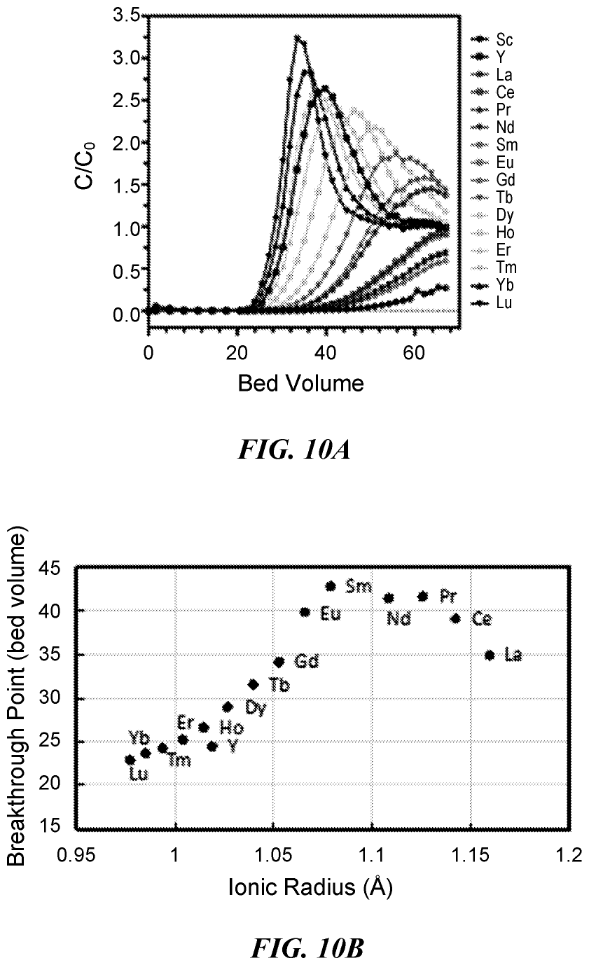

Differential affinity of LanM among REEs. To identify the REE selectivity preference of immobilized LanM, a synthetic solution containing all REEs (except Pm) was used for a column breakthrough test (FIGS. 10A and 10B). No REE eluted until the column reached the breakthrough point determined by single element experiments. Breakthrough occurred in the order of Lu, Yb, Tm, Y, Er, Ho, Dy, Tb, Gd, La, Ce, Eu, Nd, Pr, Sm, and Sc, revealing a preference towards middle-light REE over heavy REE, which is in agreement with binding affinity determinations for the solubilized protein. Interestingly, in the breakthrough region, the effluent concentrations of the heavy rare earths were higher than those of their feed concentrations suggestive of competitive displacement by REEs with higher affinity for LanM. This differential affinity of LanM among REEs can be exploited for REE separation.

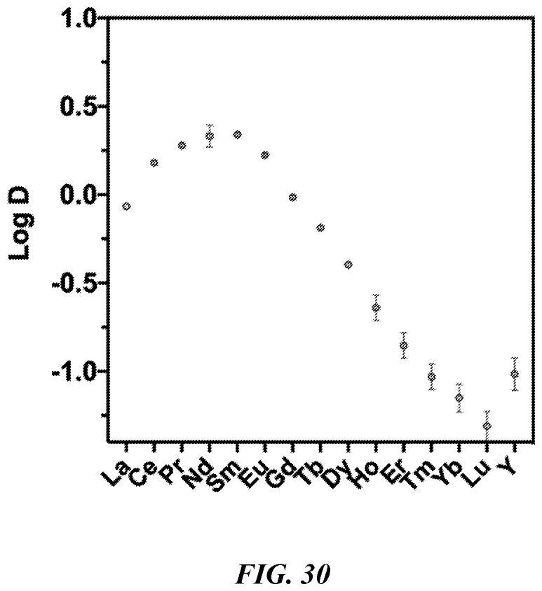

The intra-REE selectivity of lanmodulin was also quantified. Separation factors were determined by quantifying the equilibrium distribution of REEs between solution and immobilized lanmodulin (FIG. 30). More specifically, the REE series was split into two solutions with equimolar concentrations of REEs, which were flowed over a column containing immobilized lanmodulin in circular fashion until equilibrium was reached. Subsequently, the metal ion concentrations in the solution before and after adsorption were quantified. Overall, the data reveal a strong preference of lanmodulin for light-middle REEs over heavy REEs, which is in strong agreement with the competition breakthrough curve data in FIG. 10 and the binding affinity determinations for the free protein. Notable differences in distribution coefficients were observed between adjacent and middle REEs (SF of 1.4-1.8 for adjacent metal ions, Table 1, below), highlighting the potential to employ lanmodulin for the separation of these elements. In contrast, the minimal difference in distribution coefficients for Ce to Eu (SF less than or equal to 1.3 for any pair in this region), suggests that separation processes that rely exclusively on lanmodulin's affinity for REEs are unlikely to be effective for separation of these REEs.

TABLE 1
Separation factors_{(1/2)} for REE series after one adsorption cycle.
Determined from distribution coefficients from FIG. 30.

| | | 1 | | | | | | | | | | | | | | |
|---|---|---|---|---|---|---|---|---|---|---|---|---|---|---|---|---|
| | | La | Ce | Pr | Nd | Sm | Eu | Gd | Tb | Dy | Ho | Er | Tm | Yb | Lu | Y |
| 2 | D | 0.9 | 1.5 | 1.9 | 2.1 | 2.2 | 1.7 | 0.9 | 0.6 | 0.4 | 0.2 | 0.1 | 0.1 | 0.1 | 0.0 | 0.1 |
| La | 0.9 | | | | | | | | | | | | | | | |
| Ce | 1.5 | 0.6 | | | | | | | | | | | | | | |
| Pr | 1.9 | 0.5 | 0.8 | | | | | | | | | | | | | |
| Nd | 2.1 | 0.4 | 0.7 | 0.9 | | | | | | | | | | | | |
| Sm | 2.2 | 0.4 | 0.7 | 0.9 | 1.0 | | | | | | | | | | | |
| Eu | 1.7 | 0.5 | 0.9 | 1.1 | 1.3 | 1.3 | | | | | | | | | | |
| Gd | 0.9 | 0.9 | 1.6 | 2.1 | 2.3 | 2.4 | 1.8 | | | | | | | | | |
| Tb | 0.6 | 1.3 | 2.3 | 2.9 | 3.3 | 3.4 | 2.6 | 1.4 | | | | | | | | |
| Dy | 0.4 | 2.1 | 3.7 | 4.6 | 5.2 | 5.3 | 4.1 | 2.3 | 1.6 | | | | | | | |
| Ho | 0.2 | 3.8 | 6.6 | 8.3 | 9.4 | 9.6 | 7.3 | 4.1 | 2.8 | 1.8 | | | | | | |
| Er | 0.1 | 6.1 | 10.8 | 13.6 | 15.4 | 15.7 | 12.0 | 6.6 | 4.6 | 2.9 | 1.6 | | | | | |
| Tm | 0.1 | 9.2 | 16.3 | 20.4 | 23.1 | 23.5 | 18.0 | 9.9 | 7.0 | 4.4 | 2.5 | 1.5 | | | | |
| Yb | 0.1 | 12.2 | 21.5 | 26.9 | 30.4 | 31.0 | 23.7 | 13.1 | 9.2 | 5.8 | 3.2 | 2.0 | 1.3 | | | |
| Lu | 0.0 | 17.5 | 31.0 | 38.8 | 43.9 | 44.7 | 34.2 | 18.9 | 13.2 | 8.4 | 4.7 | 2.9 | 1.9 | 1.4 | | |
| Y | 0.1 | 8.9 | 15.7 | 19.7 | 22.3 | 22.7 | 17.4 | 9.6 | 6.7 | 4.3 | 2.4 | 1.4 | 1.0 | 0.7 | 0.5 | |

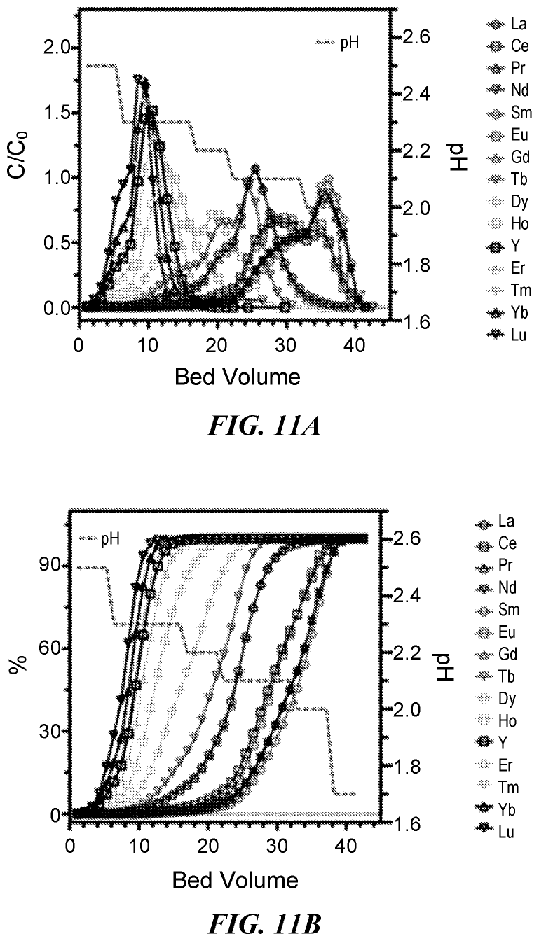

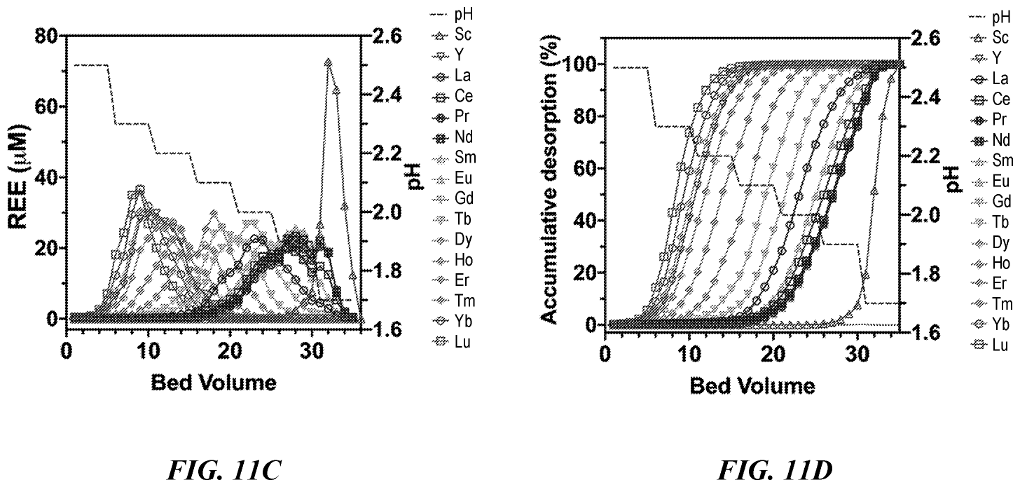

Guided by the differential breakthrough of REEs, the possibility of using pH-step to selectively desorb REE groups from a REE loaded LanM column was explored. To perform such experiment under practical condition, LanM column was pre-loaded with a synthetic REE solution that resembles the REE composition in coal fly ash leachate and then employed a multi-step pH desorption (FIGS. 11A-11D). In particular, for equimolar REE ion solution, 13 μM of each REE (Lu, Yb, Tm, Y, Er, Ho, Dy, Tb, Gd, La, Ce, Eu, Nd, Pr, Sm, and Sc) was dissolved in a pH 3, 10 mM glycine solution. The REE solution was pumped at 0.5 mL/min through a LanM column, and the column effluent was collected in 1.0 mL aliquots. The REE concentrations in effluents were determined by ICP-MS. The REE eluate was divided into three groups: heavy REEs (Ho—Lu+Y), middle REEs (Gd−Dy)+La, and light REEs (Ce−Eu). Heavy REEs desorbed first within the pH 2.3-2.5 range, whereas all light REEs, except La, were retained on column until the pH reached pH 2.1 and below. Middle REEs eluted within an intermediate pH range. These results suggested that co-extracted REEs from coal fly ash leachate (or other ore-based feedstock leachates) could be separated into three sub-groups in a single adsorption/desorption cycle. It is likely that the REEs in separated groups can be further separated using additional adsorption/desorption cycles with the LanM column.

### Separation of REEs Using Strategic Selection of Organic Chelators

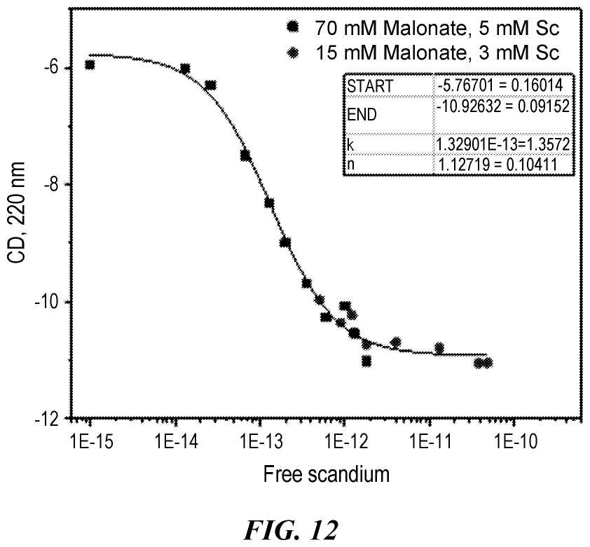

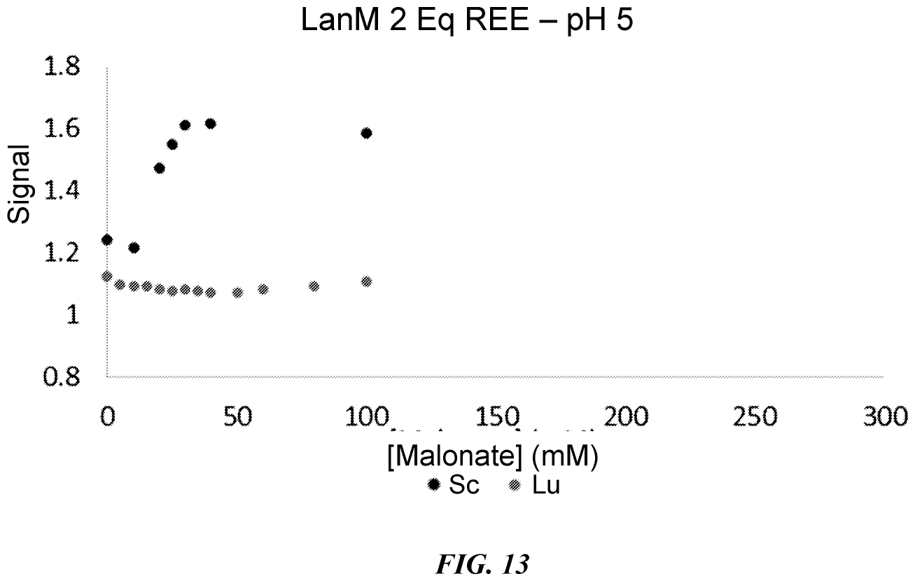

LanM enables Sc separation from REEs. LanM binds Sc with sub-picomolar apparent dissociation constant (FIG. 12). During this experiment, [6]222 nm was monitored at various malonate-buffered free Sc^{III} concentrations using CD spectroscopy, and data were fitted to the Hill equation. The Hypspec computer program⁴⁰ was used to calculate the speciation diagram for various Sc-malonate systems in 20 mM acetate, 100 mM KCl, pH 5. The derived free-metal concentrations were used in the affinity determination using a standard CD titration protocol. Sc-malonate did not contribute to the signal, so only one blank was required. Strikingly, data with free LanM indicates that the chelator malonate can strip Sc from the LanM-Sc complex at concentrations that do not affect heavy REE-LanM binding (FIG. 13). As such, malonate could be employed to separate Sc with high purity from a REE feedstock leachate by loading the LanM column with REEs in an initial adsorption step and then using malonate to selectivity recover Sc. Subsequent desorption steps with other chelators or through pH modulation can then be employed for further separation of REEs.

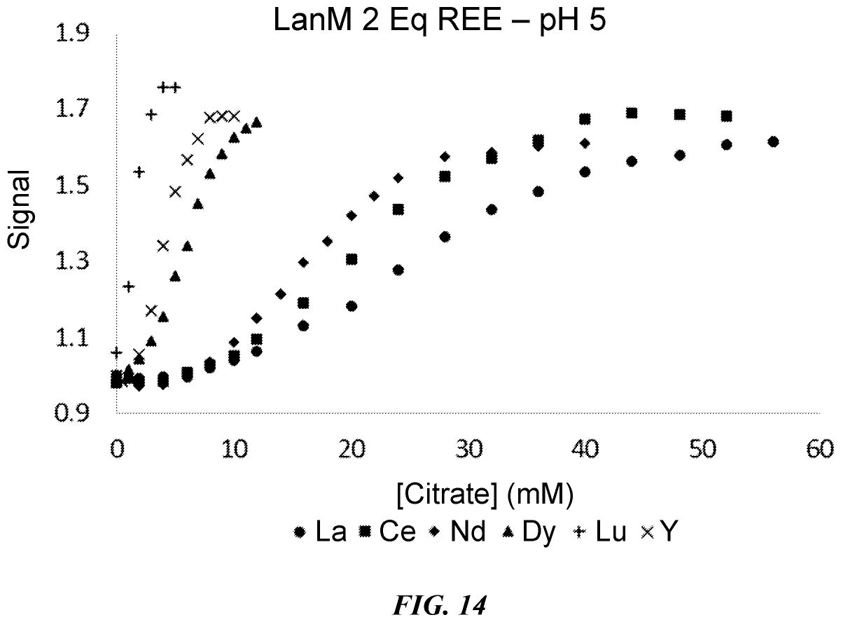

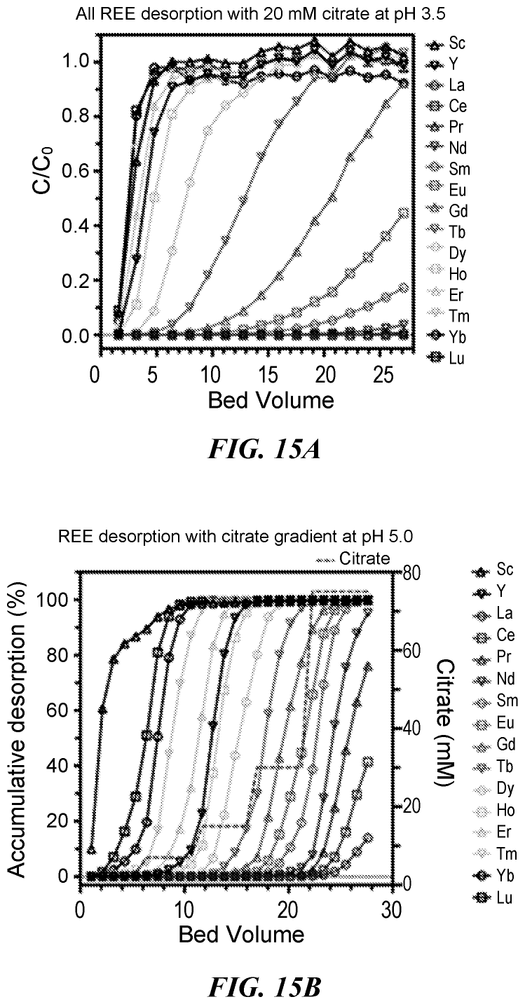

Effect of citrate on REE breakthrough and desorption. Based on the promising citrate desorption data for free LanM (FIG. 14), the effect of citrate on REE breakthrough was investigated. A synthetic solution containing all REEs (except Pm) was prepared in 20 mM citrate at pH 3.5 for a column breakthrough test. The breakthrough order of REE elution was correlated with the ionic radius. (FIG. 15A) HREEs (Lu—Tb, Y, Sc) broke through with in first 5 bed volumes whereas LREEs (La—Nd) did not breakthrough until 25+ bed volumes. Guided by the equimolar REE breakthrough experiment, the possibility of using citrate steps to selectively desorb REE from a REE loaded LanM column was explored. (FIG. 15B). As expected, the REE desorption order of REE elution also followed the ionic radius. However, surprisingly, the vast majority of Sc (80%) was desorbed by dilute citrate (3 mM) before all other REEs including Lu. This data, along with previous work with free LanM, suggests that co-extracted Sc can be effectively separated from all other REEs by using a dilute chelator solution. The use of malonate in place of citrate may allow for even cleaner separation of Sc from REEs (FIGS. 15A and 15B). The data also suggest that a staged desorption process with citrate can be employed to generate separate HREE, MREE, and LREE pools.

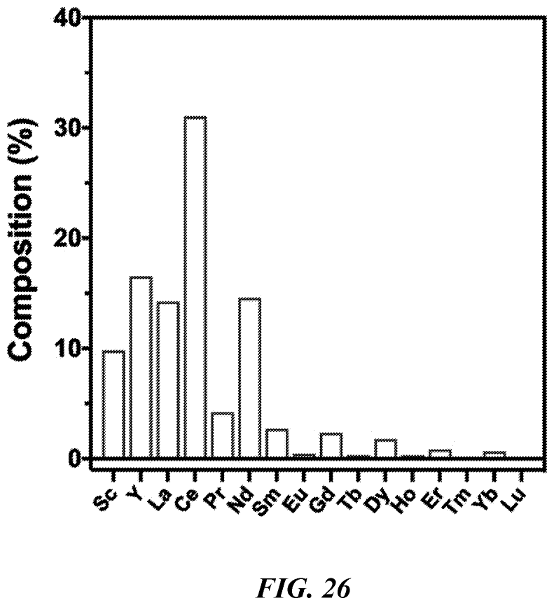

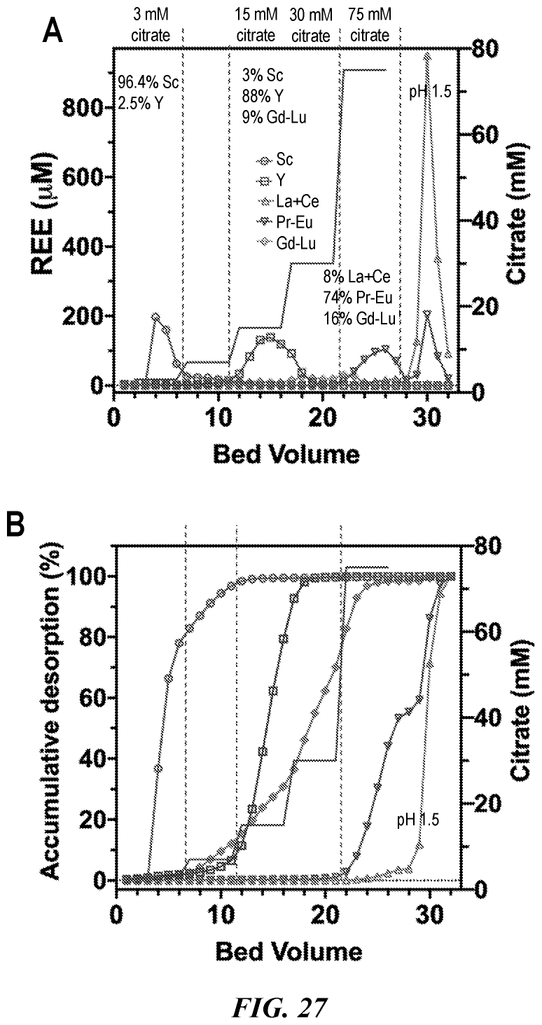

Sc/Y separation from a synthetic ore leachate by citrate-pH step desorption. A synthetic REE solution that resembles the REE composition in an ore-based feedstock leachate solution (e.g., bauxite and its waste residues, allanite, coal and coal combustion products; FIG. 26) was used to develop a desorption-based process to separate Sc and Y from the lanthanides. Given the preferential binding of citrate for Sc and Heavy-REE combined with the preferential binding of Light-Medium-REE by lanmodulin (LanM), a desorption scheme using multiple citrate steps was tested to determine whether that scheme could be used to separate the Sc and Y from the L-MREE. Work presented in FIG. 15B revealed that citrate (pH 5) concentrations of 3 mM and 15 mM were effective in desorption of Sc and Y, respectively, from the LanM column. Further, a 3 mM citrate desorption step yielded a Sc solution with purity of 96.4% and 77% recovery yield. FIG. 27 shows the desorption profiles (A) and cumulative yields (B). A subsequent desorption step using 15-30 mM citrate, yielded a Y solution with 88% purity and >90% recovery yield. A high value MREE+Nd/Pr enriched fraction was generated next using 75 mM citrate. The remaining REEs were desorbed using a pH 1.5 solution, yielding a La/Ce enriched solution. These results indicate that Sc and Y can be effectively removed from the adsorbed lanthanide fraction by using a citrate-pH desorption strategy.

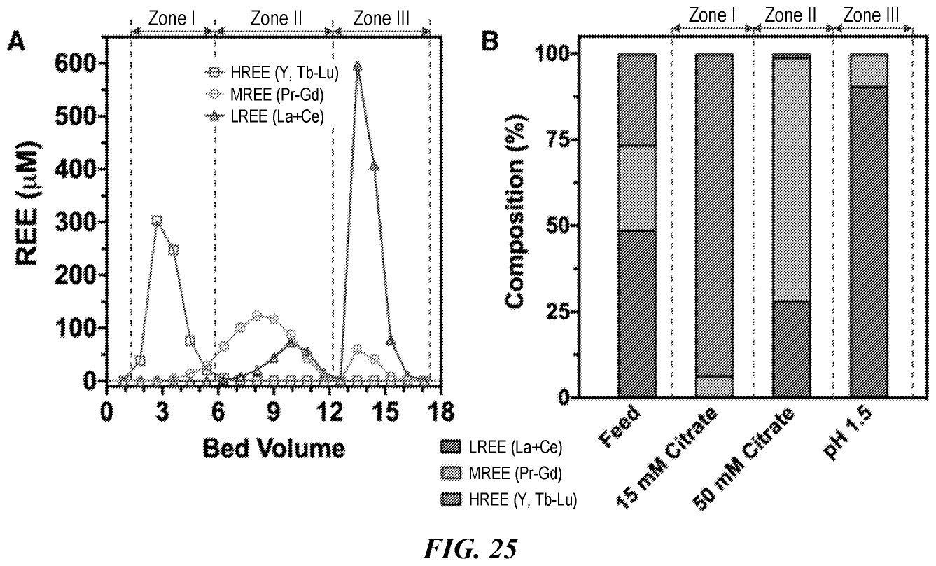

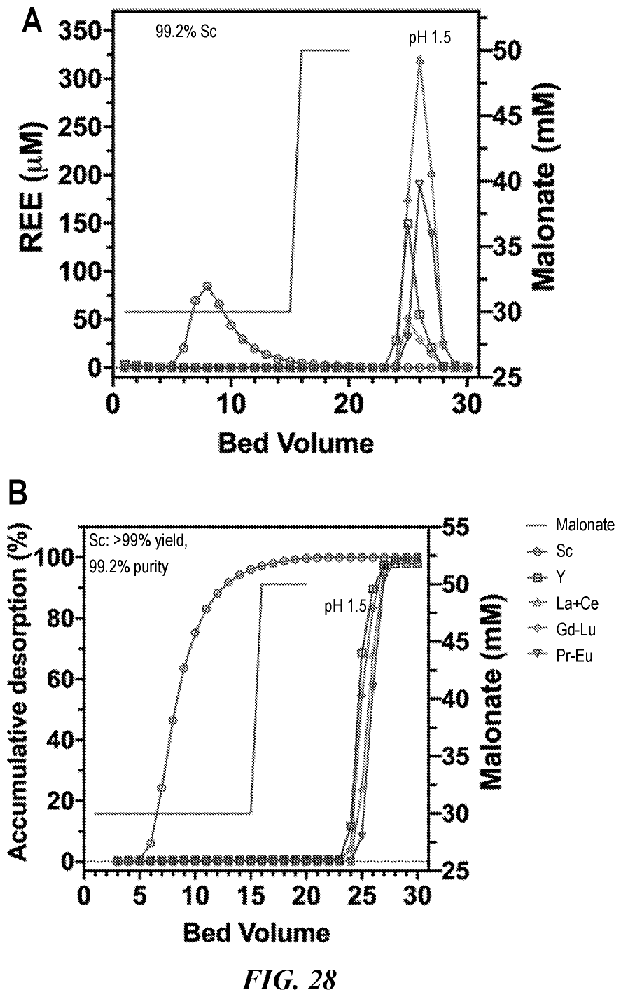

Sc/Y separation from a synthetic ore leachate by malonate desorption. Given the promising Sc/Lu separation results with free lanmodulin protein and malonate chelator depicted in FIG. 13, the efficacy of malonate to achieve high-purity Sc separation from REEs was tested. The LanM-column was loaded with an ore-based feedstock leachate solution (e.g., bauxite and its waste residues, allanite, coal and coal combustion products; FIG. 26) and subjected to desorption using increasing concentrations of malonate. The data suggest that 30 mM malonate is sufficient to separate Sc in high-purity and yield (>99% purity; >99% yield) from the REE-loaded lanmodulin column (FIG. 28). Based on the data in FIGS. 25 and 27, subsequent citrate desorption steps can likely be employed to yield Y-enriched, MREE-enriched, and La/Ce-enriched fractions. In sum, these data highlight the ability of lanmodulin to yield high-purity Sc solutions when coupled with desorbents, such as carboxylate-containing chelators.

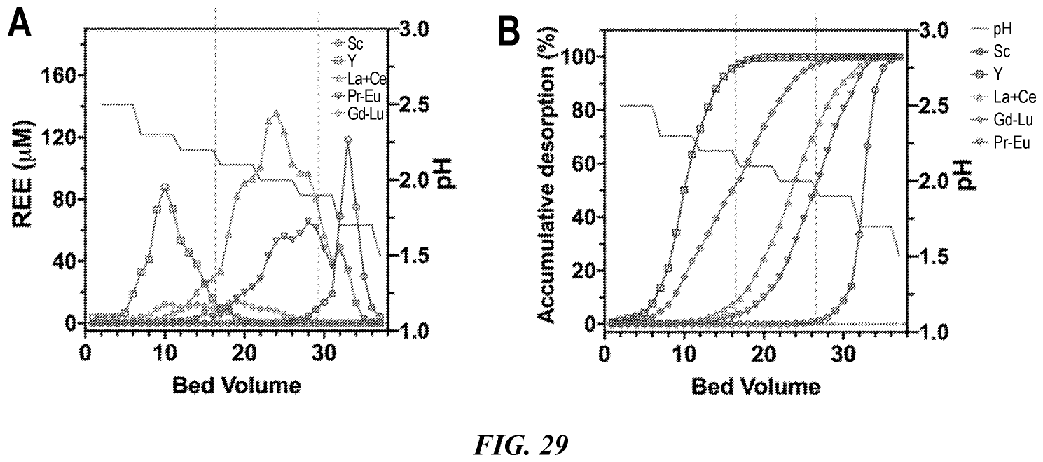

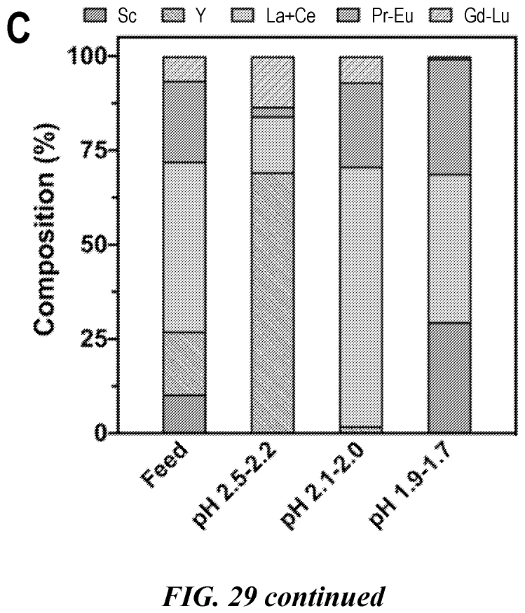

pH-based desorption for Sc separation. Given the preferential binding of lanmodulin for Sc over REEs, a desorption scheme using pH was tested to determine whether that scheme can be used for Sc separation. To test this, the LanM-column was loaded with a synthetic REE solution that resembled the REE composition in a typical Sc containing ore-based feedstock leachate solution (e.g., bauxite and its waste residues, allanite, coal and its resulting combustion products). Using a stepwise decrease in pH, it was observed that Sc is eluted in the last fraction with a purity of 30% (FIG. 29). Since the impurities are LREE and MREEs rather than HREEs, Sc can be easily separated in high purity in a subsequent step using a chelator (e.g., malonate or citrate) and following the logic from above.

Separation of grouped REEs Using Organic Chelators

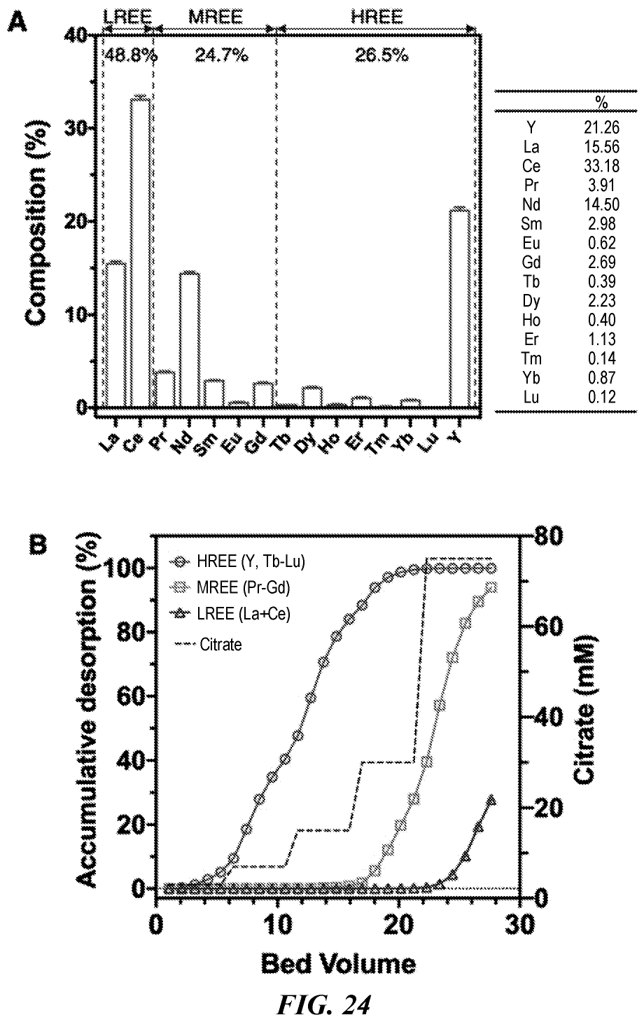

Based on the differential desorption experiments presented above (FIG. 7A and FIG. 15A-B), citrate was identified as a promising candidate for separating light/middle/heavy REEs. To determine the most effective strategy for desorption-based REE separation between LREE/MREE/HREE, the LanM-column was loaded with a synthetic REE solution that resembled the REE composition in a typical ore-based feedstock leachate solution (FIG. 24A; LREE(La+Ce): MREE(Pr—Gd): HREE(Y, Tb−Lu)=˜50%: 25%: 25%) and employed a step-wise citrate desorption scheme (FIG. 24B). LanM has similarly high affinity for middle and light REEs, precluding the use of pH-based desorption gradient for MREE v LREE separation. Given the higher affinity of citrate for MREE over LREE, a desorption scheme using citrate was tested to determine whether that scheme could be used to separate LREE/MREE/HREE into discrete groups. It was found that HREE can be selectively separated from MREE and LREE using 15 mM citrate, whereas a citrate concentration of 25-50 mM could effectively separate MREE from LREE.

After determining the optimal citrate concentration for REE separation, a follow-up experiment was performed to demonstrate grouped REE separation via a stepwise citrate-pH scheme (FIG. 25A-B). The HREEs were nearly quantitatively desorbed in high purity (HREE purity of 93.6%) using a 15 mM citrate desorption step (HREE purity of 93.6%). Subsequently, a 50 mM citrate step produced a MREE-enriched solution with purity of 70.7%. Lastly, a non-selective pH 1.5 desorption step desorbed the remaining REE, producing a concentrated La/Ce solution (purity of 90.6%). The results suggest that light REE (La and Ce) can be effectively depleted from HREE and MREE in a single citrate-based desorption step when starting with a synthetic REE composition (representative of that in a primary ore). As such, the LanM-based approach enables REE vs. non-REE separation (adsorption step) and grouped HREE/MREE/LREE separation (desorption step) in a single adsorption/desorption process.

This separation effect would also likely be observed with other soluble organic chelators such as iminodiacetic acid (IDA), ethylenediaminetetraacetic acid (EDTA), diethylenetriaminepentaacetic acid (DTPA), [S,S]-ethylenediamine-N,N′-diglutaric acid (EDDG), etc. Chelators such as malonate and HIBA, while effective for Sc and HREE desorption, have insufficient MREE affinity to compete with LanM at reasonable concentrations.

### CONCLUSION

The following example demonstrated a biomaterial based, all-aqueous REE extraction and separation platform using a REE-selective protein chelator. This concept represents a crucial step towards sustainable REE production and minimizing global dependence on primary REE resources. A recently discovered REE-binding protein chelator, LanM, was immobilized onto a bio-renewable, agarose support resin to enable flow through REE extraction and facile reuse. Immobilized LanM retained its remarkable REE selectivity and facilitated near quantitative REE separation from non-REE impurities. Followed by sequential pH gradient or mild chelator treatment, co-extracted Nd/Dy—the most critical REE pair for E-waste recovery—were separated to high purity (99.9% purity) within 1 or 2 adsorption/desorption cycles depending on the feed ratio. The study further demonstrated the application of LanM for REE extraction and separation into heavy and light REE groups in a single adsorption/desorption cycle. A key advantage of the disclosed process over the prior art is the compatibility with low-grade feedstock leachates, the lack of organic solvents, the ability to achieve high-purity separation of certain REEs while using the entire column capacity, compatibility with acidic feedstocks, high selectivity toward REE during the first adsorption step, allowing minimization of the downstream process, and no concentration of radioactive impurities (e.g., U and Th).

### Example 2: Improving the Carbon Atom Economy and Loading Capacity of LanM Column

As discussed in Example 1, a 2:1 stoichiometry of Nd per immobilized LanM was observed. Interestingly, in solution, LanM binds 3 equivalent REEs, suggesting that one metal site is destabilized upon immobilization, or in the column format. This result suggested that there was potential to shorten the protein (i.e., by removing the nonfunctional binding site) without perturbing the 2:1 stoichiometry. Shortening the protein length without perturbing the 2:1 stoichiometry can increase the overall immobilization rate of LanM on the column, and consequently the amount of REE that can be loaded on the column, thereby increasing the efficacy of REE separation from REE containing material.

The Amino Acid Sequence of LanM can be Shortened without Perturbing the REE:LanM Stoichiometry for Immobilized LanM

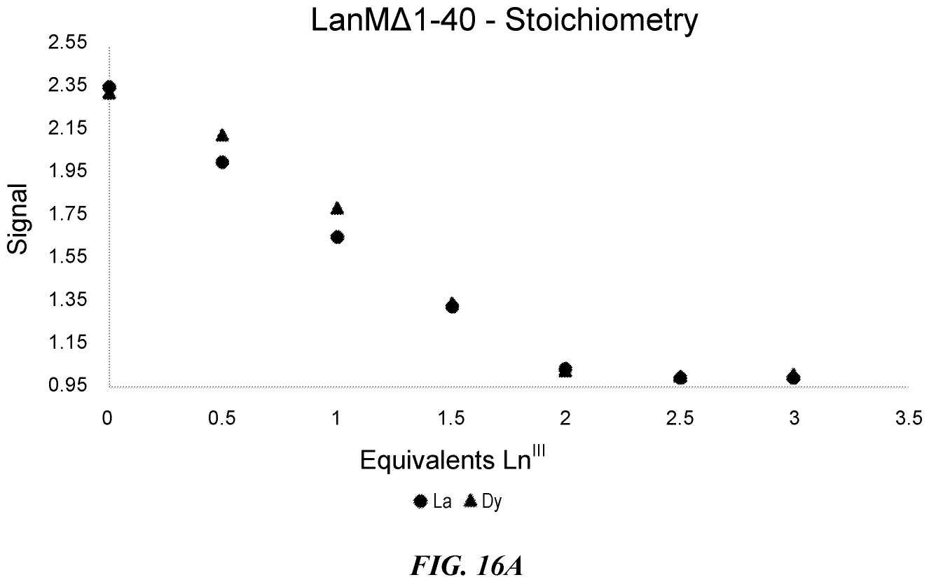

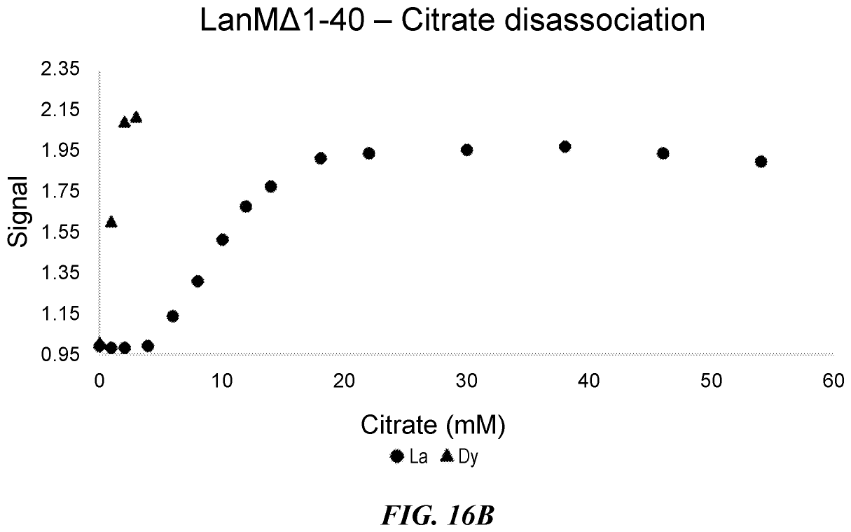

Removal of the first 40 amino acids of LanM (i.e. 19 amino acids from the N-terminus of SEQ ID NO:1) yielded a functional protein with a 10% reduction in molecular weight and a 2:1 stoichiometry of REE to LanM (FIGS. 16A and 16B) and, unexpectedly, improved the Dy/La separation prospects (FIGS. 16A and 16B); Dy could be completely recovered from LanM at citrate concentrations that did not cause disassociation of the LanM-La complex. This highlights the potential for high-purity MREE v. LREE separation using LanM α1-40 (Table 1, SEQ ID NO:2).

### Double LanM Protein

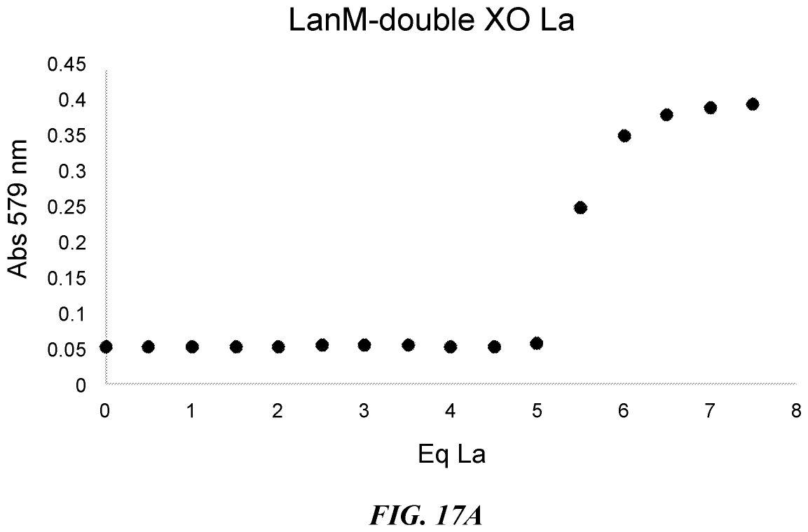

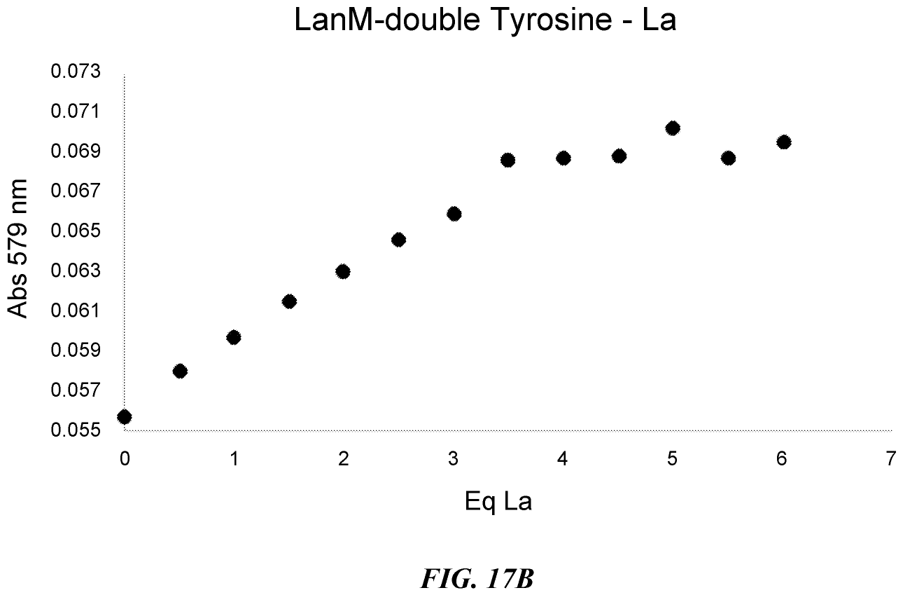

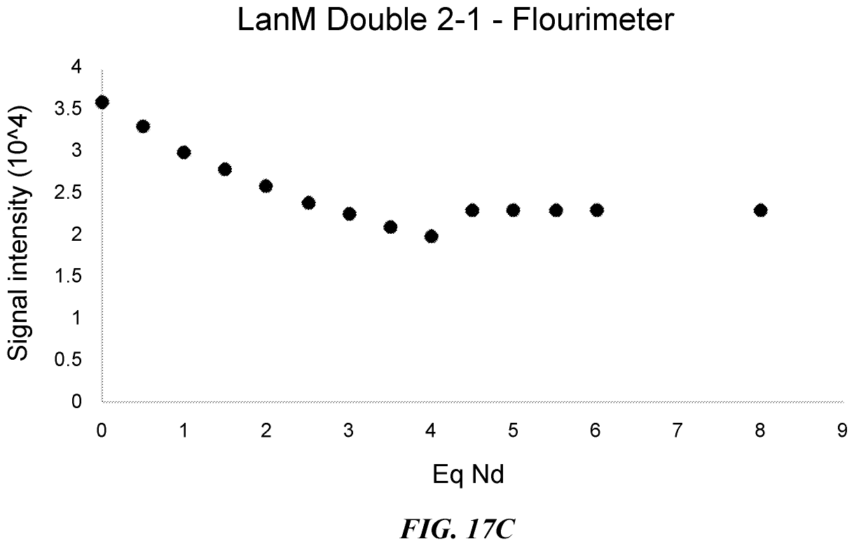

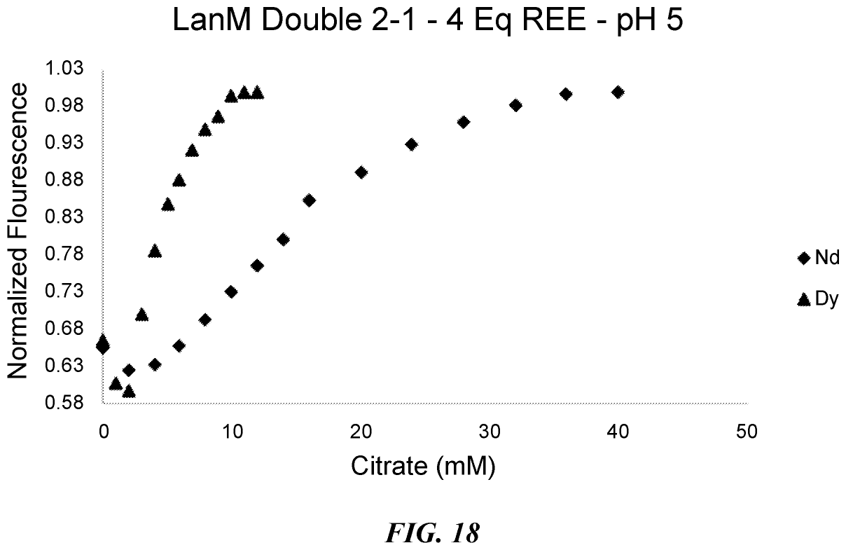

To increase the REE adsorption capacity of the LanM column, a double LanM construct was generated (called LanM-double (2-1), Table 1, SEQ ID NO:3) and the REE binding stoichiometry was tested. The stoichiometry is ˜4:1 to 5:1 in solution (i.e., the third binding site of 1 of the LanM units appears functional), but is likely to be 4:1 when immobilized based on the 2:1 stoichiometry of immobilized single LanM (FIGS. 17A-17C). The LanM-double protein will be appended with a terminal cysteine and immobilized on agarose resin for subsequent tests. Importantly, the Nd/Dy disassociation characteristics of double LanM were very similar to WT LanM (FIG. 18). This suggests that double LanM is likely to enable on column Dy separation from Nd using citrate as a desorbent.

### Example 3: Continuous HREE/LREE Separation by Using a Three-Column Rotation Scheme

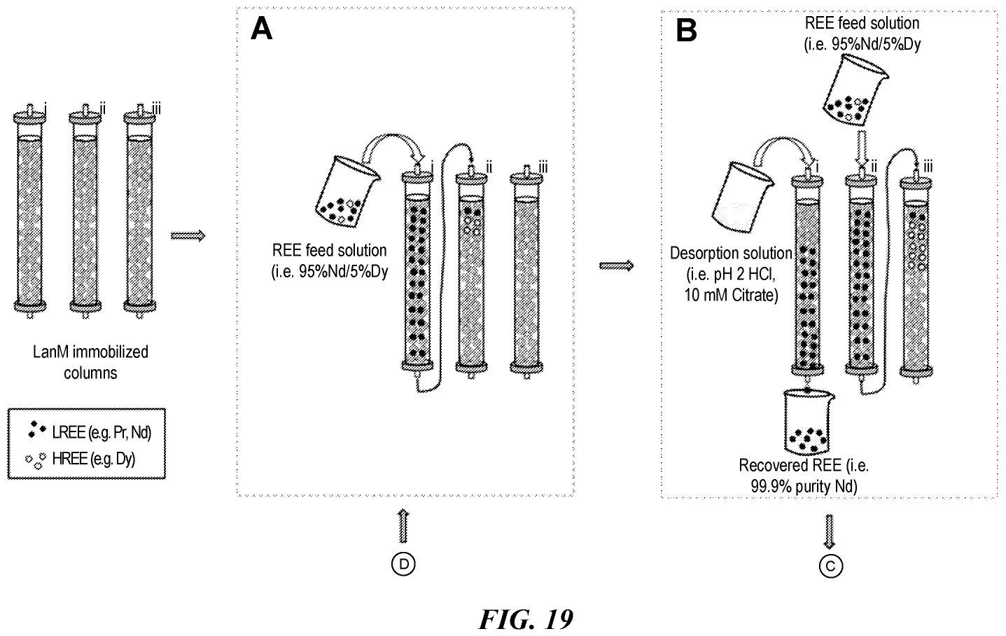

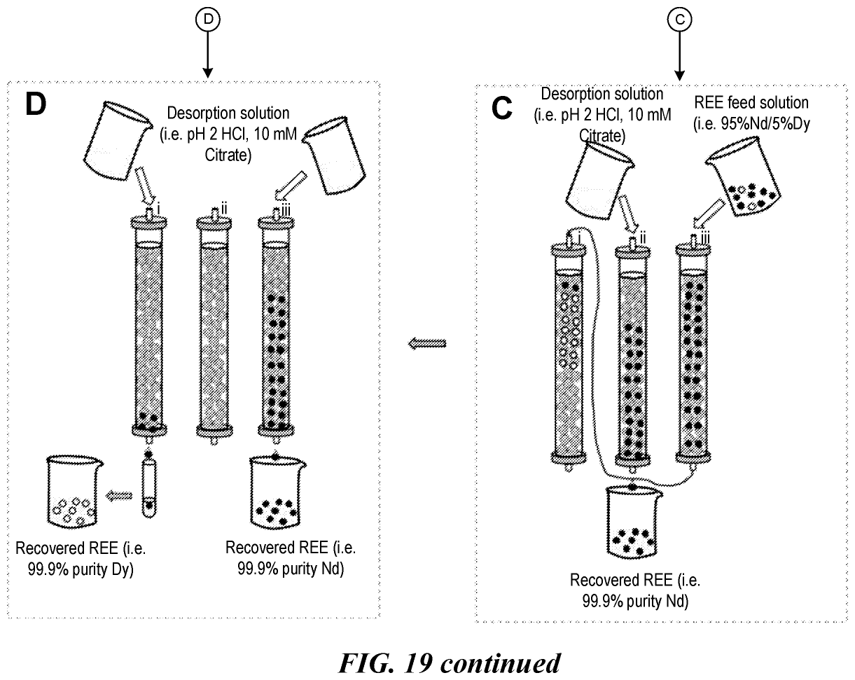

The following example describes a continuous method for REE separation by using three LanM columns in rotation as shown in FIG. 19. Such scheme includes 4 steps (step A-D) to complete a separation cycles, allowing high efficient, high yield REE separation (i.e. single REE product with >99.9% purity with >99% yield). Methods

Econo-Column glass chromatography columns (Bio-Rad; 5 cm×0.5 cm) were filled with DI-water before LanM-microbeads were added gravimetrically. Columns were washed with 25 mM HCl, DI-water and conditioned with DI-water before breakthrough experiments. REE feed solution (39 μM Al, 2.3 μM Mn, 3.1 mM Fe, 54 M Co, 2.5 μM Cu, 5.6 μM Zn, 34.9 μM Pr, 371 μM Nd, and 21.8 μM Dy) was prepared by using biolixiviant to leachate REEs from electronic waste (E-waste). The feed was pumped through column at 0.5 mL/min and effluents were collected in 1.0 mL aliquots. The REE concentrations in effluents were determined by ICP-MS.

### Results and Discussion

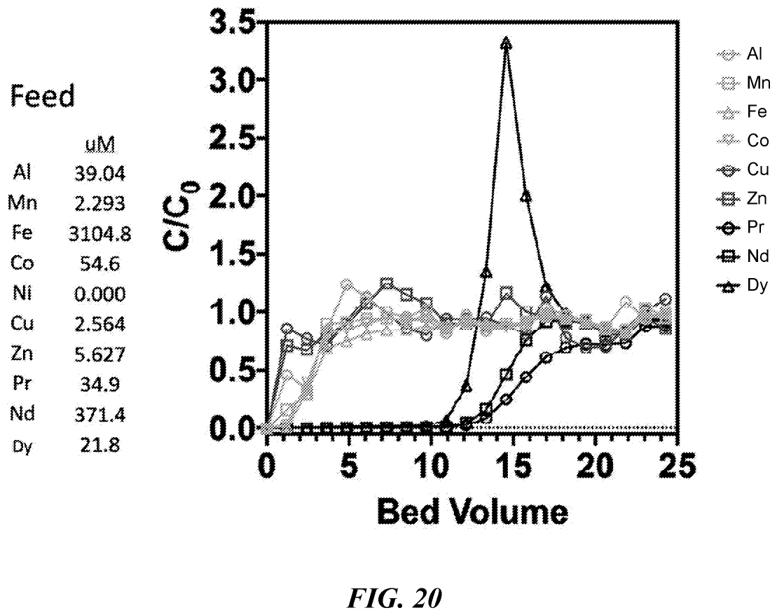

In step A, REE solution, for example Nd and Dy at pH >2.5 condition, is pumped through column i and effluents is collected by column ii. Before reaching the full capacity of column i, both Nd and Dy are extracted by column i, which is supported by previous breakthrough experiment as shown in FIG. 20 (bed volumes 1-10). However, once column i is exhausted (after bed volume 10), Dy ions will be selectively displaced by Nd due to LanM's higher LREE selectivity over HREE. Step 1 will be finished when all Dy ions are displaced by Nd ions in column i, allowing Nd enrichment in column i. It is worth noting that Dy ions are collected in column ii. In step B, the previously extracted Nd in column i will be desorbed through either low pH or mild chelator to recover high purity Nd (99.9%) and regenerate column i for following adsorption. Meanwhile, column ii and column iii are connected in series to repeat the Nd enrichment in column ii and Dy enrichment in column iii until column ii is fully loaded with Nd. In step C, the same desorption and Nd/Dy enrichment processes are repeated as described in step B with rotated column order. In step D, Dy and Nd will be recovered, respectively. Then step A will be repeated to start a new separation cycle. Notably, the process can be fully continuous if a column iv is added, in which case the Dy loaded column can be swapped with column iv to perform Dy desorption independently and that Dy desorption can be performed less frequently depending on the Nd/Dy ratio.

From the foregoing, it will be appreciated that specific embodiments of the invention have been described herein for purposes of illustration, but that various modifications may be made without deviating from the scope of the invention. Accordingly, the invention is not limited except as by the appended claims.

### REFERENCES

1. Cheisson, T.; Schelter, E. J., Rare earth elements: Mendeleev's bane, modern marvels. Science 2019, 363 (6426), 489.
2. Marx, J.; Schreiber, A.; Zapp, P.; Walachowicz, F., Comparative Life Cycle Assessment of NdFeB Permanent Magnet Production from Different Rare Earth Deposits. Acs Sustain Chem Eng 2018, 6 (5), 5858-5867.
3. Sprecher, B.; Xiao, Y.; Walton, A.; Speight, J.; Harris, R.; Kleijn, R.; Visser, G.; Kramer, G. J., Life Cycle Inventory of the Production of Rare Earths and the Subsequent Production of NdFeB Rare Earth Permanent Magnets. Environ Sci Technol 2014, 48 (7), 3951-3958.
4. Wang, L.; Huang, X.; Yu, Y.; Zhao, L.; Wang, C.; Feng, Z.; Cui, D.; Long, Z., Towards cleaner production of rare earth elements from bastnaesite in China. J Clean Prod 2017, 165, 231-242.
5. Xie, F.; Zhang, T. A.; Dreisinger, D.; Doyle, F., A critical review on solvent extraction of rare earths from aqueous solutions. Minerals Engineering 2014, 56, 10-28.
6. Vahidi, E.; Zhao, F., Environmental life cycle assessment on the separation of rare earth oxides through solvent extraction. Journal of Environmental Management 2017, 203, 255-263.
7. Lee, J. C. K.; Wen, Z., Rare Earths from Mines to Metals: Comparing Environmental Impacts from China's Main Production Pathways. Journal of Industrial Ecology 2017, 21 (5), 1277-1290.
8. El-Sofany, E. A., Removal of lanthanum and gadolinium from nitrate medium using Aliquat-336 impregnated onto Amberlite XAD-4. Journal of Hazardous Materials 2008, 153 (3), 948-954.
9. Nagaphani Kumar, B.; Radhika, S.; Ramachandra Reddy, B., Solid-liquid extraction of heavy rare-earths from phosphoric acid solutions using Tulsion CH-96 and T-PAR resins. Chem Eng J 2010, 160 (1), 138-144.
10. Horwitz, E. P.; Chiarizia, R.; Dietz, M. L.; Diamond, H.; Nelson, D. M., Separation and preconcentration of actinides from acidic media by extraction chromatography. Anal Chim Acta 1993, 281 (2), 361-372.
11. Florek, J.; Chalifour, F.; Bilodeau, F.; Larivi6re, D.; Kleitz, F., Nanostructured Hybrid Materials for the Selective Recovery and Enrichment of Rare Earth Elements. Advanced Functional Materials 2014, 24 (18), 2668-2676.
12. Zhang, H.; McDowell, R. G.; Martin, L. R.; Qiang, Y., Selective Extraction of Heavy and Light Lanthanides from Aqueous Solution by Advanced Magnetic Nanosorbents. Acs App/Mater Inter 2016, 8 (14), 9523-9531.
13. Hu, Y.; Drouin, E.; Larivi6re, D.; Kleitz, F.; Fontaine, F.-G., Highly Efficient and Selective Recovery of Rare Earth Elements Using Mesoporous Silica Functionalized by Preorganized Chelating Ligands. Acs App/Mater Inter 2017, 9 (44), 38584-38593.
14. Hu, Y.; Misal Castro, L. C.; Drouin, E.; Florek, J.; Kshlig, H.; Larivi6re, D.; Kleitz, F.; Fontaine, F.-G., Size-Selective Separation of Rare Earth Elements Using Functionalized Mesoporous Silica Materials. Acs App/Mater Inter 2019, 11 (26), 23681-23691.
15. Hu, Y.; Florek, J.; Larivi6re, D.; Fontaine, F.-G.; Kleitz, F., Recent Advances in the Separation of Rare Earth Elements Using Mesoporous Hybrid Materials. The Chemical Record 2018, 18 (7-8), 1261-1276.
16. Deblonde, G. J. P.; Mattocks, J. A.; Park, D. M.; Reed, D. W.; Cotruvo, J. A.; Jiao, Y., Selective and Efficient Biomacromolecular Extraction of Rare-Earth Elements using Lanmodulin. Inorg Chem 2020, 59 (17), 11855-11867.
17. Gupta, N. K.; Gupta, A.; Ramteke, P.; Sahoo, H.; Sengupta, A., Biosorption-a green method for the preconcentration of rare earth elements (REEs) from waste solutions: A review. Journal of Molecular Liquids 2019, 274, 148-164.
18. Park, D. M.; Reed, D. W.; Yung, M. C.; Eslamimanesh, A.; Lencka, M. M.; Anderko, A.; Fujita, Y.; Riman, R. E.; Navrotsky, A.; Jiao, Y. Q., Bioadsorption of Rare Earth Elements through Cell Surface Display of Lanthanide Binding Tags. Environ Sci Technol 2016, 50 (5), 2735-2742.
19. Park, D. M.; Brewer, A.; Reed, D. W.; Lammers, L. N.; Jiao, Y. Q., Recovery of Rare Earth Elements from Low-Grade Feedstock Leachates Using Engineered Bacteria. Environ Sci Technol 2017, 51 (22), 13471-13480.
20. Cotruvo, J. A.; Featherston, E. R.; Mattocks, J. A.; Ho, J. V.; Laremore, T. N., Lanmodulin: A Highly Selective Lanthanide-Binding Protein from a Lanthanide-Utilizing Bacterium. Journal of the American Chemical Society 2018, 140 (44), 15056-15061.
21. Awadhiya, A.; Kumar, D.; Rathore, K.; Fatma, B.; Verma, V., Synthesis and characterization of agarose-bacterial cellulose biodegradable composites. Polymer Bulletin 2017, 74 (7), 2887-2903.
22. Hermanson, G. T., Chapter 6 —Heterobifunctional Crosslinkers. In Bioconjugate Techniques (Third Edition), Hermanson, G. T., Ed. Academic Press: Boston, 2013; pp 299-339.
23. Nair, D. P.; Podg6rski, M.; Chatani, S.; Gong, T.; Xᵢ, W.; Fenoli, C. R.; Bowman, C. N., The Thiol-Michael Addition Click Reaction: A Powerful and Widely Used Tool in Materials Chemistry. Chem Mater 2014, 26 (1), 724-744.
24. Judge, W. D.; Azimi, G., Recent progress in impurity removal during rare earth element processing: A review. Hydrometallurgy 2020, 196, 105435.
25. Byrne, R. H.; Li, B., Comparative complexation behavior of the rare earths. Geochim Cosmochim Ac 1995, 59 (22), 4575-4589.
26. Mattocks, J. A.; Ho, J. V.; Cotruvo, J. A., A Selective, Protein-Based Fluorescent Sensor with Picomolar Affinity for Rare Earth Elements. Journal of the American Chemical Society 2019, 141 (7), 2857-2861.
27. Yang, Y.; Walton, A.; Sheridan, R.; Guth, K.; GauB, R.; Gutfleisch, O.; Buchert, M.; Steenari, B.-M.; Van Gerven, T.; Jones, P. T.; Binnemans, K., REE Recovery from End-of-Life NdFeB Permanent Magnet Scrap: A Critical Review. Journal of Sustainable Metallurgy 2017, 3 (1), 122-149.
28. Sun, P.-P.; Kim, D.-H.; Cho, S.-Y., Separation of neodymium and dysprosium from nitrate solutions by solvent extraction with Cyanex272. Minerals Engineering 2018, 118, 9-15.
29. Yoon, H. S.; Kim, C. J.; Chung, K. W.; Kim, S. D.; Kumar, J. R., Process development for recovery of dysprosium from permanent magnet scraps leach liquor by hydrometallurgical techniques. Canadian Metallurgical Quarterly 2015, 54 (3), 318-327.
30. Hussain, R.; Luo, K., Geochemical Evaluation of Enrichment of Rare-Earth and Critical Elements in Coal Wastes from Jurassic and Permo-Carboniferous Coals in Ordos Basin, China. Natural Resources Research 2020, 29 (3), 1731-1754.
31. Taggart, R. K.; Hower, J. C.; Dwyer, G. S.; Hsu-Kim, H., Trends in the Rare Earth Element Content of U.S.-Based Coal Combustion Fly Ashes. Environ Sci Technol 2016, 50 (11), 5919-5926.
32. Ye, Q.; Li, G.; Deng, B.; Luo, J.; Rao, M.; Peng, Z.; Zhang, Y.; Jiang, T., Solvent extraction behavior of metal ions and selective separation Sc3+ in phosphoric acid medium using P204. Separation and Purification Technology 2019, 209, 175-181.
33. Yagmurlu, B.; Dittrich, C.; Friedrich, B., Effect of Aqueous Media on the Recovery of Scandium by Selective Precipitation. Metals-Basel 2018, 8 (5), 314.
34. Middleton, A.; Park, D. M.; Jiao, Y.; Hsu-Kim, H., Major element composition controls rare earth element solubility during leaching of coal fly ash and coal by-products. International Journal of Coal Geology 2020, 227, 103532.
35. Park, D.; Middleton, A.; Smith, R.; Deblonde, G.; Laudal, D.; Theaker, N.; Hsu-Kim, H.; Jiao, Y., A biosorption-based approach for selective extraction of rare earth elements from coal byproducts. Separation and Purification Technology 2020, 241, 116726.
36. Smith, R. C.; Taggart, R. K.; Hower, J. C.; Wiesner, M. R.; Hsu-Kim, H., Selective Recovery of Rare Earth Elements from Coal Fly Ash Leachates Using Liquid Membrane Processes. Environ Sci Technol 2019, 53 (8), 4490-4499.
37. Zhang, N.; Li, H. X.; Liu, X. M., Recovery of scandium from bauxite residue-red mud: a review. Rare Metals 2016, 35 (12), 887-900.
38. Huang, X.; Dong, J.; Wang, L.; Feng, Z.; Xue, Q.; Meng, X., Selective recovery of rare earth elements from ion-adsorption rare earth element ores by stepwise extraction with HEH(EHP) and HDEHP. Green Chem 2017, 19 (5), 1345-1352.
39. Featherston, E. R.; Mattocks, J. A.; Tirsch, J. L.; Cotruvo, J. A., Chapter Six-Heterologous expression, purification, and characterization of proteins in the lanthanome. In Methods in Enzymology, Cotruvo, J. A., Ed. Academic Press: 2021; Vol. 650, pp 119-157.
40. Gans P.; Sabatini, A; and Vacca, A, Investigation of equilibria in solution. Determination of equilibrium constants with the HYPERQUAD suite of programs, Talanta, 1996; Vol. 43, pp. 1739-1753.

1-96. (canceled)

97. A method for separating rare earth elements (REE) from non-REE elements present in a REE containing material comprising the steps of:
a) providing a protein that can selectively bind one or more REEs;
b) contacting an amount of the protein with the REE-containing material, wherein the protein binds at least a portion of the one or more REEs to form one or more protein-REE complexes and an REE-depleted material;
c) separating the one or more protein-REE complexes from at least a portion of the REE-depleted material; and
d) separating the one or more REEs from the protein to produce a purified fraction of the one or more REEs and a regenerated protein.

98. The method of claim 97, further comprising reusing the regenerated protein to carry out steps (a)-(d).

99. The method of claim 97, wherein the separation step (c) separates the one or more REEs from the REE containing material.

100. The method of claim 97, wherein the separating step (c) preferentially separates one or more individual REEs, groups of REEs, REEs adjacent to each other on the periodic table, REEs having similar ionic radii or combination thereof.

101. The method of claim 97, wherein the protein does not bind non-REEs in the REE containing material.

102. The method of claim 97, wherein the protein is LanM.

103. The method of claim 102, wherein LanM comprises an amino acid sequence selected from the group consisting of SEQ ID NO:1, SEQ ID NO:2, SEQ ID NO:3, and SEQ ID NO:4.

104. The method of claim 97, wherein LanM comprises an amino acid sequence with at least 95% identity to a sequence selected from the group consisting of SEQ ID NO:1, SEQ ID NO:2, SEQ ID NO:3, and SEQ ID NO:4.

105. The method of claim 104, wherein the LanM includes a C-terminal cysteine residue, a N-terminal cysteine residue, or an internal cysteine residue.

106. The method of claim 105, wherein the cysteine residue is linked to the LanM via a glycine serine amino acid linker.

107. The method of claim 97, wherein the protein binds at least a portion of the one or more REEs in step (c) at a pH of 2 to about 10.

108. The method of claim 97, wherein the REE is separated from the protein at a pH of less than about 2 in step (d).

109. The method of claim 97, wherein the REE is separated from the protein using a water-soluble organic chelator in step (d).

110. The method of claim 97, wherein the REE material is a leachate derived from rare earth ores, geothermal brines, coal, coal byproducts, mine tailings, acid mine drainage, phosphogypsum, end of life products, electronic waste, industrial effluent, or total REE mixtures generated from REE sources, such as salts derived from these ores (e.g. oxide, oxalate, carbonate, etc).

111. The method of claim 1, wherein the REE containing material is a low-grade material, a high-grade material, or a combination thereof.

112. The method of claim 97, wherein the protein that can selectively bind an REE is bound to a porous support material functionalized with a conjugation agent; and wherein the method further comprises a step of conjugating the protein to the porous support material via the conjugation agent.

113. The method of claim 16, wherein the porous support material is an agarose microbead.

114. A method of preferentially separating scandium and yttrium from a REE-containing material, the method comprising steps of:
(a) providing a plurality of proteins that can selectively bind one or more REEs;
(b) contacting the plurality of proteins with the REE-containing material, wherein the plurality of proteins bind at least a portion of the one or more REEs to form a plurality of protein-REE complexes and an REE-depleted material;
(c) separating the plurality of protein-REE complexes from at least a portion of the REE-depleted material;
(d) preferentially separating scandium from the plurality of proteins by contacting the plurality of protein-REE complexes with a first chelator solution;
(e) preferentially separating yttrium from the plurality of proteins by contacting the plurality of protein-REE complexes with a second chelator solution;
(f) separating heavy (HREEs) and middle REEs (MREEs) from the plurality of proteins by contacting the plurality of protein-REE complexes with a solution comprising a third chelator solution; and
(g) separating light REEs (La and Ce) from the plurality of proteins by contacting the plurality of protein-REE complexes with a fourth chelator solution or a solution having a low pH (<1.7).

115. The method of claim 93, wherein first solution includes malonate at a concentration of about 20-50 mM, the second solution contains citrate at a concentration of about 15.0 mM, the third solution includes citrate at a concentration of about 25 mM to about 50 mM, and the fourth solution has a pH of about 1.5.

116. A method of preferentially separating scandium from a REE-containing material, the method comprising steps of
(a) providing a plurality of proteins that can selectively bind one or more REEs;
(b) contacting the plurality of proteins with the REE-containing material, wherein the plurality of proteins bind at least a portion of the one or more REEs to form a plurality of protein-REE complexes and an REE-depleted material;
(c) separating the plurality of protein-REE complexes from at least a portion of the REE-depleted material;
(d) preferentially separating yttrium and HREEs from the plurality of proteins by contacting the plurality of protein-REE complexes with a first pH solution;
(e) preferentially separating MREEs and LREEs from the plurality of proteins by contacting the plurality of protein-REE complexes with a second pH solution;
(f) separating scandium from the plurality of proteins by contacting the plurality of protein-REE complexes with a solution comprising a third pH solution.


SEQUENCE LISTING (4 sequences)

SEQ ID NO: 1 - protein, 112 aa, Methylobacterium extorquens
  MISC_FEATURE (1)..(112): wild type
APTTTTKVDIAAFDPDKDGTIDLKEALAAGSAAFDKLDPDKDGTLDAKELKGRVSEADLKKLDPDNDGTLDKKEYLAAVEAQFKAANPDNDGTIDARELASPAGSALVNLIR

SEQ ID NO: 2 - protein, 116 aa, Artificial Sequence
  synthetic construct
APTTTTKVDIAAFDPDKDGTIDLKEALAAGSAAFDKLDPDKDGTLDAKELKGRVSEADLKKLDPDNDGTLDKKEYLAAVEAQFKAANPDNDGTIDARELASPAGSALVNLIRGSGC

SEQ ID NO: 3 - protein, 99 aa, Artificial Sequence
  synthetic construct
TIDLKEALAAGSAAFDKLDPDKDGTLDAKELKGRVSEADLKKLDPDNDGTLDKKEYLAAVEAQFKAANPDNDGTIDARELASPAGSALVNLIRHHHHHH

SEQ ID NO: 4 - protein, 249 aa, Artificial Sequence
  synthetic construct
APTTTTKVDIAAFDPDKDGTIDLKEALAAGSAAFDKLDPDKDGTLDAKELKGRVSEADLKKLDPDNDGTLDKKEYLAAVEAQFKAANPDNDGTIDARELASPAGSALVNLIRGSGAEAAAKEAAAKEAAAKEAAAKAAPTTTTKVDIAAFDPDKDGTIDLKEALAAGSAAFDKLDPDKDGTLDAKELKGRVSEADLKKLDPDNDGTLDKKEYLAAVEAQFKAANPDNDGTIDARELASPAGSALVNLIR